(19) United States
(12) Patent Application Publication
(10) Pub. No.: US 2025/0381210 A1
(43) Pub. Date: Dec. 18, 2025

(54) METHODS OF TREATING VASCULAR DEMENTIA

(71) Applicant: The Regents of the University of California, Oakland, CA (US)

(72) Inventors: Stanley Carmichael, Sherman Oaks, CA (US); Min Tian, Los Angeles, CA (US)

(21) Appl. No.: 19/242,440

(22) Filed: Jun. 18, 2025

Related U.S. Application Data
(60) Provisional application No. 63/661,439, filed on Jun. 18, 2024.

(51) Int. Cl. A61K 31/7076; A61P 25/28

(52) U.S. Cl. CPC A61K 31/7076; A61P 25/28

(57) ABSTRACT

The disclosure provides methods of treating ischemic disease by modulating A3AR or Serpine2 activity.

### RELATED APPLICATIONS

This application claims the benefit of U.S. Provisional Patent Application No. 63/661,439, filed Jun. 18, 2024, which is hereby incorporated by reference in its entirety.

### GOVERNMENT SUPPORT

This invention was made with government support under NS102185 awarded by the National Institutes of Health. The government has certain rights in the invention.

### BACKGROUND

Vascular dementia (VaD) is a white matter ischemic disease and the second-leading cause of dementia, with no direct therapy. VaD produces disability and reduces quality of life, with a rising incidence due to an aging population. VaD arises from compromised cerebral blood flow, particularly ischemia and/or infarction in WM, which are often initially asymptomatic, resulting in tissue damage and cognitive decline. Within the lesion site, cell-cell interactions dictate the trajectory towards disease progression or repair. Unlike Alzheimer's disease with established transgenic models, VaD lacks suitable counterparts, hindering therapeutic progress. Methods of treating vascular dementia are needed.

### SUMMARY OF THE INVENTION

In certain aspects, the present disclosure provides methods of treating or preventing an ischemic disease, the method comprising administering to a subject an A3AR agonist, or a pharmaceutically acceptable salt thereof.

In further aspects, the present disclosure provides methods of treating or preventing an ischemic disease, the method comprising modulating Serpine2 activity.

### BRIEF DESCRIPTION OF THE DRAWINGS

FIG. 1A shows Circos plots showing the intercellular interactome in mouse and human VaD. Signaling from microglia to other cells that are changed in mouse VaD, identified by mouse pool of L-R library.

FIG. 1B shows signaling from microglia to other cells that are changed in human VaD, identified by mouse pool of L-R library.

FIG. 1C shows signaling from EC to other cells that are changed in mouse VaD, identified by human pool of L-R library.

FIG. 1D shows signaling from EC to other cells that are changed in mouse VaD, identified by mouse pool of L-R library.

FIG. 1E shows signaling from EC to other cells that are changed in human VaD, identified by human pool of L-R library.

FIG. 1F shows signaling from EC to other cells that are changed in human VaD, identified by mouse pool of L-R library.

FIG. 1G shows signaling from pericyte to other cells that are changed in mouse VaD, identified by human pool of L-R library.

FIG. 1H shows signaling from pericyte to other cells that are changed in mouse VaD, identified by mouse pool of L-R library.

FIG. 1I shows signaling from mural cell to other cells that are changed in human VaD, identified by human pool of L-R library.

FIG. 1J shows signaling from mural cell to other cells that are changed in human VaD, identified by mouse pool of L-R library.

FIG. 1K shows signaling from OPC to other cells that are changed in mouse VaD, identified by human pool of L-R library.

FIG. 1L shows signaling from OPC to other cells that are changed in mouse VaD, identified by mouse pool of L-R library.

FIG. 1M shows signaling from OPC to other cells that are changed in human VaD, identified by human pool of L-R library.

FIG. 1N shows signaling from OPC to other cells that are changed in human VaD, identified by mouse pool of L-R library.

FIG. 1O shows signaling from astrocyte to other cells that are changed in mouse VaD, identified by human pool of L-R library.

FIG. 1P shows signaling from astrocyte to other cells that are changed in mouse VaD, identified by mouse pool of L-R library.

FIG. 1Q shows signaling from astrocyte to other cells that are changed in human VaD, identified by human pool of L-R library.

FIG. 1R shows signaling from astrocyte to other cells that are changed in human VaD, identified by mouse pool of L-R library. The Log fold change of each L/R gene is indicated in the gradient color box. The interacting cells are connected by arrows through L-R pairs.

FIG. 2A shows RIN value, unique mapped reads (%), and number of reads for RNA-Seq samples. The values for EC samples.

FIG. 2B shows the values for pericyte samples.

FIG. 2C shows the values for OPC samples.

FIG. 2D shows the values for astrocyte samples. Each dot represents an individual animal.

FIG. 3A shows mouse model that mimics vascular dementia (VaD) in human. Schematic shows the induction of VaD in mouse cerebral white matter (WM) by 3 intracranial injections of vasoconstrictor L-NIO; lesion location highlighted in maroon. The right panel shows the hyperintensity (indicated with white arrows) in MRI images (ischemia) from VaD mouse model. Scale bars=1 mm.

FIG. 3B shows representative immunostaining images comparing changes in five WM cell types in VaD (lower panels) with control brain tissue (upper panels). From left to right, the images show: microglia (Iba1), endothelial cells (ECs) (CD31), pericytes/fibroblasts (CD13), oligodendrocyte lineage (OL) (Olig2), astrocytes (GFAP). Cell nuclei are counterstained with DAPI. Scale bar=50 μm. For all quantification figures, each dot represents an individual animal, and data are presented as mean±SEM unless otherwise stated.

FIG. 3C shows quantification of Iba1 (microglia) immunoreactivity as a percentage (%) of WM volume within the region of interest. VaD vs Con, ****p<0.0001. t test.

FIG. 3D shows the change of vessel diameter (μm) measured by the immunostaining of CD31 (EC) and CD13 (pericyte). CD31, VaD vs Con, *p<0.05; CD13, VaD vs Con, **p<0.01. t test.

FIG. 3E shows quantification of CD13 (pericytes and fibroblasts) immunoreactivity as a percentage (%) of WM volume within the region of interest. VaD vs Con, **p<0.01. t test.

FIG. 3F shows changes in the number of oligodendrocyte lineage cells (Olig2+). VaD vs Con, *p<0.05. t test.

FIG. 3G shows quantification of GFAP (astrocyte) immunoreactivity as a percentage (%) of WM volume within the region of interest. VaD vs Con, ***p<0.001. t test.

FIG. 3H shows single channel immunostaining images in unilateral VaD mouse model shows the boundary of infarct core defined by demyelination (degradation of MBP), axon damage (breakdown of NF160), and astrocyte activation. Asterisk indicates the core of ischemic lesion in VaD. Dashed lines show the boundary of lesion core. Scale bar=100 μm. DAPI, nucleus.

FIG. 3I shows quantification of lesion core size at 1-month time point post VaD induction.

FIG. 3J shows schematic of the novel object recognition test (NOR).

FIG. 3K shows quantification of novel object preference (%). Con vs. VaD, *p<0.05, t test. 1-month post VaD induction.

FIG. 3L shows regression of novel object preference (%) against lesion size (mm³). Simple linear regression, p=0.01, r2=0.696. 1-month post VaD induction.

FIG. 3M shows schematic shows the paradigm of memory linking test.

FIG. 3N shows quantification of freezing (%) in associated (Ctx A), novel (gray), and shocked (Ctx B,) contexts. Con (Ctx A) vs VaD (Ctx A), *p<0.05; Con (Ctx A) vs Con (novel), ***p<0.001; VaD (Ctx A) vs VaD (novel), **p<0.01. Two-way repeated measures ANOVA.

FIG. 3O shows schematic shows the retrograde labeling of hippocampus (HPC) projecting neurons from prefrontal cortex (PFC) by injections of retroAAV-PGK-Cre virus in HPC, and AAV1-CAG-Flex-EGFP in PFC.

FIG. 3P shows an immunostaining image shows the labeling of GFP, and Cre recombinase in prefrontal cortex including the anterior cingulate (AC) subregion. Scale bar=50 μm. Dashed lines show the border of PFC subregions.

FIG. 3Q shows immunostaining images with single channels show the colocalization of Cre expression and retrogradely labeled neurons by GFP. DAPI. Scale bar=25 μm.

FIG. 3R shows schematic shows the region of infarct core in VaD model, and images show virus labeled axon bundles (GFP) passing the regions in control and VaD WM. Asterisk indicates the core of infarct lesion. Scale bar=100 μm.

FIG. 3S shows quantification results of axon volume labeled with GFP in WM, % of GFP+ axon volume/WM volume. Con vs. VaD, *p<0.05, t test.

FIG. 3T shows representative images show the axon processes (labeled with GFP, indicated with arrow heads) in the HPC in control and VaD brains. Scale bar=100 μm.

FIG. 3U shows quantification results of axon volume in HPC labeled with GFP, % of GFP+ axon volume/HPC volume. Con vs. VaD, *p<0.05, t test.

FIG. 3V shows schematic shows the test of neuronal activity in PFC-HPC circuits related to memory retrieval using fear conditioning test and immediate early gene (c-Fos) staining.

FIG. 3W shows representative images show the difference of c-Fos signal in AC, hippocampus (including CA1, CA2/3, DG subregions) in control and VaD brains. DAPI. Scale bar=100 μm.

FIG. 3X shows quantification of c-Fos cells in AC. Con vs. VaD, *p<0.05, t test.

FIG. 3Y shows quantification of c-Fos cells in HPC. CA2/3, Con vs. VaD, *p<0.05, t test.

FIG. 4A shows sustained memory and motor deficits, as well as the progressive nature of the VaD mouse model. quantification of object preference (%) in NOR test shows the memory deficit in VaD mice compared with control mice at 1-month post VaD induction. Familiar vs Novel in control group, ***, p<0.001. Familiar vs Novel in VaD group, n.s.=not significant. Two-way ANOVA. For all quantification figures, each dot represents an individual animal, and data are presented as mean±SEM unless otherwise stated.

FIG. 4B shows quantification of object preference (%) in NOR test shows that the memory deficit persists to 2-month post VaD induction. Familiar vs Novel in control group, ****, p<0.0001. Familiar vs Novel in VaD group, n.s.=not significant. Two-way ANOVA.

FIG. 4C shows grid walking test results at different time point (baseline at 1-day before, 7-day post, 30-day post, and 60-day post) shows the persistence of motor deficits in VaD mouse model. Con vs VaD on day 7, *p<0.05; Con vs VaD on day 30, *p<0.05; Con vs VaD on day 60, **p<0.01. Two-way ANOVA. Twelve animals per group.

FIG. 4D shows representative image shows the immunostaining of Satb2, Ctip2 in Layer I-VI, and schematic shows the region of brain (dashed box) for quantification of Satb2+ and Ctip2+ cells. CC, corpus callosum. Scale bars=100 μm. DAPI.

FIG. 4E shows representative images show the immunostaining of Satb2, Ctip2 in Layer VI in control and VaD brains. Scale bars=100 μm.

FIG. 4F shows quantification results of Satb2+ and Ctip2+ cell numbers in Layer VI. Satb2, Con vs VaD, ***p<0.001; Ctip2, Con vs VaD, n.s.=not significant. t test.

FIG. 4G shows representative images show the immunostaining of Cux1 and NeuN in Layer VI in control and VaD brains. DAPI. Scale bars=100 μm.

FIG. 4H shows quantification results of Cux1+, NeuN+, and DAPI+ cell numbers in Layer VI. Cux1, Con vs VaD, *p<0.05; NeuN and DAPI, Con vs VaD, n.s.=not significant. t test.

FIG. 4I shows progressive reduction of Satb2 expression in distal Layer VI in VaD mouse brains. Quantification results of Satb2/DAPI density in motor-somatosensory cortex, 0-100, 100-200, 200-300, and 300-400 μm away from WM ischemic lesion core in VaD mouse model. 0-100 μm: 6 h vs naïve, ****, p<0.0001; 6 h vs 3 d, 7 d, 2 w, or 4 w, n.s.=not significant. 100-200 μm: **, 6 h vs naïve, p<0.01; 6 h vs 3 d, 7 d, 2 w, or 4 w, n.s.=not significant. 200-300 μm: 6 h vs naïve, 6 h vs 3 d, n.s.=not significant; 6 h vs 7 d, *p<0.05; 6 h vs 2 w, ***p<0.001, 6 h vs 4 w, **p<0.01. 300-400 μm: 6 h vs naïve, 6 h vs 3 d, n.s.=not significant; 6 h vs 7 d, *p<0.05; 6 h vs 2 w, *p<0.05; 6 h vs 4 w, **p<0.01. One-way ANOVA. Four animals per group.

FIG. 4J shows representative images show the immunostaining of Satb2 in motor-somatosensory cortex, 200-300 and 300-400 μm away from WM, at different time points (6 hour, 3 days, 7 days, 2 weeks, and 4 weeks) post VaD induction and in naïve brain. DAPI. Scale bars=100 μm.

FIG. 5A shows characteristics of mouse VaD model. Immunostaining of mouse IgG showing the damage of blood brain barrier 1-week post VaD induction in mouse cerebral WM. CC, corpus callosum; V, ventricle; scale bar=100 μm.

FIG. 5B shows immunostaining image shows the injection of retroAAV-PGK-Cre in the hippocampus spread across all the subregions (CA1, CA2/3, and DG) as indicated by the expression of Cre recombinase. Scale bar=100 μm.

FIG. 5C shows representative images show the axon processes (labeled with GFP) in the HPC in control and VaD brains after retrograde AAV labeling. Scale bar=100 μm. DAPI. Arrow heads show the GFP+ axon processes.

FIG. 5D shows immunostaining images show the staining of Ctip2, Satb2 and NeuN in Layer V in control and VaD mouse brain. Ctip2; Satb2; NeuN. Scale bar=100 μm.

FIG. 5E shows quantification of Satb2+ cell numbers in Layer V. Con vs VaD, n.s.=not significant.

FIG. 5F shows quantification of Ctip2+ cell numbers in Layer V. Con vs VaD, n.s.=not significant.

FIG. 5G shows immunostaining images showing the staining of NeuN and Cux1 in Layers II/III-V in control and VaD mouse brain. NeuN; Cux1; DAPI. Scale bar=100 μm.

FIG. 5H shows quantification of Cux1+ cell numbers in Layer II/III, IV, and V. Con vs VaD, n.s.=not significant.

FIG. 5I shows quantification of NeuN+ cell numbers in Layer II/III, IV, and V. Con vs VaD, n.s.=not significant.

FIG. 5J shows summary of the VaD mouse model with human VaD pathological and clinical characteristics.

FIG. 6A shows cell type specific RNA-Seq of VaD associated DEGs in five WM cell types. Purification of EC mRNA by translating ribosome affinity purification (TRAP). Representative immunostaining image shows the specific labeling of EC by retro-orbital injection of PHP-CAG-Flex-Rpl22-HA virus into Tie2-Cre transgenic mice. Glut1, HA, DAPI; Asterisk indicates the VaD lesion core, dashed line shows the border. Scale bar=50 μm.

FIG. 6B shows purification of EC mRNA by translating ribosome affinity purification (TRAP). Real-time PCR results show the enrichment (fold change, pulldown vs input) of only EC cell type marker gene (Tek) but not microglia (Iba1), astrocyte (Gfap), pericyte/fibroblast (Pdgfrβ), or OPC (Pdgfra) markers in control and VaD. For all quantification figures, each dot represents an individual animal, and data are presented as mean±SEM unless otherwise stated. (C and D) Purification of pericyte/fibroblast mRNA by TRAP.

FIG. 6C shows representative immunostaining image shows the specific labeling of pericyte/fibroblast using transgenic Tbx18-CreER::Rpl22-HA mice. CD13, HA, DAPI; Scale bar=50 μm.

FIG. 6D shows real-time PCR results show the enrichment of only pericyte/fibroblast indicated by cell type marker gene (Pdgfrβ) but not OPC (Pdgfra), astrocyte (Gfap), microglia (Iba1), or EC (Tek) in control and VaD. (E and F) Purification of OPC mRNA by TRAP.

FIG. 6E shows representative immunostaining image shows the specific labeling of OPC and its derived oligodendrocyte lineage using transgenic Ng2-CreER::Rpl22-HA mice. Olig2, HA, DAPI; Scale bar=50 μm.

FIG. 6F shows real-time PCR results show the enrichment of only OPC indicated by cell type marker gene (Pdgfra) but not pericyte/fibroblast (Pdgfrβ), astrocyte (Gfap), microglia (Iba1), or EC (Tek) in control and VaD. (G and H) Purification of astrocyte mRNA by TRAP.

FIG. 6G shows representative immunostaining image shows the specific labeling of astrocyte by intracranial injection of Lenti-GfaABC1D-Rpl22-HA virus into C57 mice.

FIG. 6H shows real-time PCR results show the enrichment of only astrocyte indicated by cell type marker gene (Gfap) but not OPC (Pdgfra), pericyte/fibroblast (Pdgfrβ), microglia (Iba1), or EC (Tek) in control and VaD.

FIG. 6I shows schematic of how TRAP purify ribosomal mRNA using HA antibody.

FIG. 6J shows representative heatmap (from the OPC RNA-Seq results using Ng2-CreER::Rpl22-HA and TRAP) shows WM pulldown fraction after TRAP enrich/diminish canonical cell type marker genes in control and VaD. Diminished canonical cell type markers are indicated by arrows to the right.

FIG. 6K shows representative PCA result (from the OPC RNA-Seq results using Ng2-CreER::Rpl22-HA and TRAP) shows the clustering of input.Con, pulldown.Con, input. VaD, and pulldown. VaD fractions. Each dot represents an individual animal.

FIG. 6L shows schematic shows the identification of DEGs related to VaD by comparing pulldown. VaD and pulldown.Con samples in each cell type with FDR<0.1. (I-L) shows the flow of TRAP purification of EC, pericyte/fibroblast, OPC, and astrocyte mRNA to the identification of VaD related DEGs.

FIG. 6M shows schematic shows the purification of microglia cells by FACS using CX3CR1, CD11b, CD45 antibodies from WM.

FIG. 6N shows representative FACS plot shows the collection of P2 fraction as microglia sample from control brain.

FIG. 6O shows representative FACS plot shows the collection of P2 and P4 fraction as microglia samples from VaD brains.

FIG. 6P shows PCA analysis of microglia samples from control and VaD brains. Each dot represents an individual animal.

FIG. 6Q shows schematic shows the identification of microglial DEGs related to VaD by comparing VaD.P4 vs Con.P2, and VaD.P2 vs Con.P2 samples in each cell type with FDR<0.1. (M-Q) shows the flow of FACS purification of microglia RNA and identification of VaD related DEGs.

FIG. 6R shows Venn diagram shows the overlap of DEGs across 5 cell types in mouse VaD model.

FIG. 6S shows column graph shows the number of VaD associated DEGs expressed in one, or two, or multiple cell type(s) in mouse VaD model.

FIG. 6T shows Venn diagram shows the overlap of DEGs across 8 cell types in human VaD.

FIG. 6U shows column graph shows the number of VaD associated DEGs expressed in one, or two, or multiple cell type(s) in human VaD.

FIG. 7A shows cell-type specific HA labeling for TRAP, PCA analysis results, and low macrophage contamination in FACS sorted microglia. Immunostaining images show that ribosomal expression of HA, using Tie2-Cre×PHP-CAG-Flex-Rpl22-HA, does not get into microglia (Iba1), astrocyte (GFAP), pericyte/fibroblast (Pdgfrβ), or oligodendrocyte lineage (Olig2). DAPI; Scale bar=50 μm; Asterisk indicates the lesion core; Dashed line shows the border of infarct.

FIG. 7B shows immunostaining images show that pericyte/fibroblast HA labeling, using Tbx18-CreER::Rpl22-HA transgenic mice, does not get into microglia (Iba1), astrocyte (GFAP), EC (CD31), or oligodendrocyte lineage (Olig2). DAPI; Scale bar=50 μm; Asterisk indicates the lesion core; Dashed line shows the border of infarct.

FIG. 7C shows the paradigm of tamoxifen injection and VaD induction in mice cohorts for pericyte/fibroblast and OPC labeling.

FIG. 7D shows immunostaining images show that OPC HA labeling, using Ng2-CreER::Rpl22-HA transgenic mice, does not get into microglia (Iba1), astrocyte (GFAP), pericyte/fibroblast (CD13), or EC (Glut1). DAPI; Scale bar=50 μm; Asterisk indicates the lesion core; Dashed line shows the border of infarct.

FIG. 7E shows quantification of HA+Olig2+/HA+ (%) in the cerebral WM of Ng2-CreER::Rpl22-HA transgenic mice.

FIG. 7F shows schematic shows the region of cerebral WM where the oligodendrocyte lineage cells are investigated in VaD and intact conditions.

FIG. 7G shows representative images show the colocalization of HA expression with OPC (Pdgfra), total lineage (Olig2), and myelinated oligodendrocyte (Aspa+) in intact brain, infarct core, and peri-infarct WM in VaD. DAPI; Scale bar=50 μm.

FIG. 7H shows immunostaining images show that astrocyte HA labeling, using C57 mice×Lenti-GfaABC1D-Rpl22-HA, does not get into microglia (Iba1), EC (CD31), pericyte/fibroblast (CD13), or oligodendrocyte lineage (Olig2). DAPI; Scale bar=50 μm; Asterisk indicates the lesion core.

FIG. 7I shows PCA results show the clustering of input.Con, pulldown.Con, input. VaD, and pulldown. VaD fractions after TRAP enrichment of pericyte/fibroblast,

FIG. 7J shows PCA results show the clustering of input.Con, pulldown.Con, input. VaD, and pulldown. VaD fractions after TRAP enrichment of EC,

FIG. 7K shows PCA results show the clustering of input.Con, pulldown.Con, input. VaD, and pulldown. VaD fractions after TRAP enrichment of astrocyte.

FIG. 7L shows comparison of macrophage marker (Mrc1, Pf4, F13a1) and microglia marker (Tmem119, P2ry12, Cx3cr1, and Aif1) in FACS sorted cells in terms of FPKM. N=8 mice in control group, and n=9 mice in VaD groups.

FIG. 8A shows identification of WM specific transcription factors (TFs) and cell markers. A partial list of WM specific TFs in 5 cell types, comparing their normalized specificity (see STAR Methods) in WM and cortex/whole brain.

FIG. 8B shows the expression (FPKM) of example TFs in WM: Foxc1 in pericytes/fibroblasts, Myc1 in OPCs, Atoh8 in astrocytes, and Zfp871 in microglia.

FIG. 8C shows these TFs are not specific in corresponding cell types in the whole brain or cortical RNA-Seq.

FIG. 8D shows a partial list of WM specific cell type markers, comparing their normalized specificity in WM and cortex/whole brain.

FIG. 8E shows the expression (FPKM) of example WM specific cell type markers: Tmem212 in EC, Slc1a1 in OPC, Thbs4 in astrocyte and Nav3 in microglia.

FIG. 8F shows these markers are not specific in cortical cells.

FIG. 9A shows RRHO and GSEA analyses of WM RNA-Seq dataset compared with cortical/whole brain RNA-Seq datasets indicate specific transcriptomic characteristics of WM cells. Pearson correlation coefficient comparing WM with cortex cells using RRHO. WM OPC and EC are fairly distinctive from cortical OPC and EC respectively.

FIG. 9B shows Rank-Rank scatter plot shows the EC-related gene-expression signatures with medium overlap in WM and cortex.

FIG. 9C shows Pearson correlation coefficient comparing WM with whole brain cells using RRHO. White matter microglia and EC are medium to small correlation with whole brain microglia and EC respectively.

FIG. 9D shows Rank-Rank scatter plot shows the microglia-related gene-expression signatures with small overlap in WM and whole brain.

FIG. 9E shows GSEA analysis comparing EC from WM and cortical cell RNA-Seq shows the top up-regulated (left) and down-regulated (right) genes in WM. Five samples from WM and 2 samples from cortex were compared.

FIG. 9F shows GSEA analysis comparing microglia from WM and cortical cell RNA-Seq shows the top up-regulated (left) and down-regulated (right) genes in WM. Five samples from WM and 2 samples from cortex were compared.

FIG. 10A shows cell type-specific WM associated aging gene changes in VaD mouse model. Number of WM-associated aging genes that are either up- or down-regulated in VaD in 5 cell types.

FIG. 10B shows Venn graph shows the number of overlapped aging genes across 5 cell types.

FIG. 10C shows dot plots show the log fold change of aging genes in each cell type, VaD vs Con. The size of dots represents the expression level (FPKM) of genes. Venn plot in the square of Microglia_P2 shows the number of overlapping and different aging gene changes in P4 vs P2 fractions in VaD. C4b is highlighted in maroon.

FIG. 10D shows microglia KEGG pathways analysis of aging genes changed in VaD mouse model.

FIG. 10E shows microglia GO molecular function pathways analysis of aging genes changed in VaD mouse model.

FIG. 11A shows identification of ligand-receptor (L-R) interactions in VaD neurovascular niche, using custom L-R database, in mouse VaD model and human patients. Schematic of cell-cell interaction through L-R pairs in the neurovascular niche, generated by BioRender.

FIG. 11B shows a custom-made L-R pair database comprising 4,053 pairs annotated in humans and 2,032 pairs annotated in mice. The database was constructed by integrating data from three major L-R databases, with overlap and unique pair numbers indicated.

FIG. 11C shows identification of L-R genes from cell type-specific RNA-Seq of mouse VaD model and human VaD snRNA-Seq results. For example, from the mouse VaD RNA-Seq result, 118 ligands and 114 receptors were identified from microglia.

FIG. 11D shows Circos plot illustrating the intercellular interactome in the mouse VaD brain, focusing on interactions from microglia to all other cell types. Genes with a Log fold change between −1.5 and 2 were excluded to optimize clarity and space in the Circos plot, as including all genes would render gene names indiscernible. The Log fold change of each gene is represented by a gradient color scale. Interacting cells are connected by arrows, indicating specific L-R pairs.

FIG. 11E shows Circos plot shows the intercellular interactome in human VaD brain from microglia to all the cell types. The Log fold change of each gene is indicated by a gradient color scale. Interacting cells are connected by arrows, indicating specific L-R pairs.

FIG. 12A shows screening criteria for L-R candidates and the identification of VaD associated ECM/GPCR genes. The L-R pairs are classified into 6 tiers by whether the ligand is changed VaD, whether the change is in both mouse and human, whether the dysregulated ligand expression is specific in one or two cell type(s), whether the expression of corresponding receptor is also changed in mouse or human, and whether the L-R is implicated in CNS function. Samples in Tier1 are Entpd1 (↓)-Adora3 (↓) and Serpine2 (↑)-Lrp1 (↑).

FIG. 12B shows bubble plot showing the expression of ECM genes in 5 cell types. The gradient color shows how they are changed (log FC) in VaD. The bubble size shows the FPKM value of each gene in control brains.

FIG. 12C shows bubble plot showing the expression of GPCR genes in 5 cell types. The gradient color shows how they are changed (log FC) in VaD. The bubble size shows the FPKM value of each gene in control brains.

FIG. 13A shows intercellular Serpine2-Lrp1 pathway inhibits OPC differentiation in VaD tissue repair. The increase (fold change of FPKM) of Serpine2 expression in 5 cell types in VaD mouse brain. OPC, astrocyte, or EC vs pericyte/fibroblast or microglia P2 fraction, **p<0.01; OPC, astrocyte, or EC vs pericyte/fibroblast or microglia P4 fraction, ***p<0.001. Each dot represents an individual animal. One-way repeated measures ANOVA. For all quantification figures, each dot represents an individual animal, and data are presented as mean±SEM unless otherwise stated.

FIG. 13B shows RNAscope images showing the Serpine2 expression in pericyte/fibroblast (Anpep) cells in control and VaD WM. Scale bar=25 μm.

FIG. 13C shows RNAscope images showing the Serpine2 expression in microglia (Aif1) cells in control and VaD WM. Scale bar=25 μm.

FIG. 13D shows the expression of SERPINE2 in astrocyte in human periventricular WM under normal and VaD conditions. VaD_Adj, adjacent area of VaD lesion core. Con vs VaD core, p=5.43×10⁻¹²⁶; Con vs VaD Adj, p=6.86×10⁻⁴⁶.

FIG. 13E shows immunostaining images show the expression of Lrp1 in OPC in mouse WM.

FIG. 13F shows the difference of Lrp1 expression (FPKM) in OPC in control and VaD mouse brain. Con vs VaD, *p<0.05. n=6 mice per group.

FIG. 13G shows schematic shows the hypothesis of intercellular Serpine2-Lrp1 signaling from pericyte/microglia (in mouse) or astrocyte (in human) to OPC, which inhibit differentiation of OPC towards myelinated oligodendrocyte.

FIG. 13H shows representative images show the immunostaining of MBP in wild type (WT) and Serpine2+/− animals under control and VaD conditions. V, ventricle. Scale bar=100 μm.

FIG. 13I shows quantification of MBP volume in WM (dashed lines in FIG. 6H) of four animal cohorts: WT_con, WT_VaD, Serpine2+/−_Con, and +/−_VaD. WT_Con vs WT_VaD, **p<0.01; WT_VaD vs+/−_VaD, *p<0.05. Two-way repeated measures ANOVA.

FIG. 13J shows schematic shows the paradigm of EdU labeling of newborn OPCs (Olig2+EdU+) differentiated into myelinated oligodendrocytes (Aspa+Olig2+EdU+) during VaD repair.

FIG. 13K shows representative immunostaining images showing the differentiated newborn OPCs (Aspa+Olig2+EdU+) in control and VaD mouse brain. Olig2; Aspa; EdU. Triple positive cells were highlighted by cyan arrow heads. Scale bar=50 μm.

FIG. 13L shows quantification of differentiated newborn OPCs in VaD lesion core (Aspa+Olig2+EdU+/Olig2+EdU+, %). WT_Con vs WT_VaD and WT_VaD vs+/−_VaD, *p<0.05. Two-way repeated measures ANOVA.

FIG. 13M shows the result of NOR test using WT and Serpine2+/− animals. WT_Con vs WT_VaD and WT_VaD vs+/−_VaD, *p<0.05. Two-way repeated measures ANOVA.

FIG. 13N shows representative heat maps show the exploration towards novel objects in WT_Con, WT_VaD, and +/−_VaD cohorts.

FIG. 14A shows Lrp1 expression in OPC and other cell types. Immunostaining images show the Lrp1 expression in OPCs (Pdgfra). DAPI. Cyan arrow heads highlight the OPCs. Scale bar=10 μm.

FIG. 14B shows immunostaining images show similar Lrp1 expression pattern in OPCs in both Serpine2+/+ and Serpine2+/−mouse brains. Lrp1; Pdgfra; DAPI. Cyan arrow heads highlight the Lrp1 expression in OPCs. Scale bar=10 μm.

FIG. 14C shows immunostaining images show the expression of Lrp1 in neuron (NeuN) and astrocyte (GFAP) but not EC (CD31) or pericyte/fibroblast (CD13). DAPI. Scale bar=10 μm.

FIG. 15A shows intercellular CD39-A3AR signaling pathway is a potential target for enhancing VaD tissue and behavior recovery. Representative immunostaining images of CD39 showing the specific expression in microglia (Iba1), and EC (CD31) in mouse cerebral WM. DAPI; scale bar=10 μm.

FIG. 15B shows representative images showing the change of CD39 immunoreactivity in control and VaD brains at different ages (3-, 8-, 12-30-months). The arrow heads highlight the expression of CD39 in microglia, and the arrow heads highlight the expression in EC. Scale bar=50 μm.

FIG. 15C shows representative images of CD39 immunohistochemistry (IHC) in normal and VaD human brains. The arrow heads highlight the expression of CD39 in microglia, and the arrow heads highlight the expression in EC. Scale bar=100 μm.

FIG. 15D shows quantification of CD39 immunoreactivity in human periventricular WM by percentage of positive pixel within the region of interest. Con vs VaD, **p<0.01, t test. For all quantification figures, each dot represents an individual animal, and data are presented as mean±SEM unless otherwise stated.

FIG. 15E shows regression of CD39 immunoreactivity against sample ages in control and VaD brains. Simple linear regression, VaD vs Con, **p<0.01.

FIG. 15F shows schematic shows the hypothesis of intercellular CD39 (EC/microglia) to A3AR (microglia) signaling in VaD lesion and adjacent area that are downregulated due to the combined effects of infarct and aging.

FIG. 15G shows schematic shows the paradigm of microglial specific overexpression of CD39 in VaD lesion site and adjacent area by intracranial injection of AAV.MG1.2-CHB-Flex-Entpd1-HA virus into P2ry12-CreER mice brain. The effect of CD39 overexpression was validated by comparison to control group P2ry 12-CreER mice×AAV.MG1.2-CHB-Flex-V5.

FIG. 15H shows representative images show the VaD lesion sizes in mouse brains injected with control or CD39 overexpressing viruses. MBP immunostaining shows the border of demyelination; GFAP immunostaining shows the activation of astrocyte. DAPI. Scale bars=100 μm.

FIG. 15I shows quantification of lesion core sizes in VaD mouse brains injected with control or CD39 overexpressing viruses. Con vs VaD, *p<0.05, t test.

FIG. 15J shows quantification of lesion core length across the anterior-posterior axis in VaD mouse brains injected with control or CD39 overexpressing viruses. Con vs VaD, **p<0.01, t test.

FIG. 15K shows schematic shows the paradigm of piclidenoson (Pic) treatment starts from 5-days after VaD induction, and the examination of cognitive function and tissue repair 3-weeks after treatment.

FIG. 15L shows quantification of VaD lesion size. Each dot represents an individual animal. Veh vs Pic, *p<0.05, t test.

FIG. 15M shows quantification of axon volume as evaluated by NF160 immunostaining. Veh vs Pic, n.s.=not significant.

FIG. 15N shows quantification of myelin volume as evaluated by MBP immunostaining. Veh vs Pic, *p<0.05, t test.

FIG. 15O shows schematic of the NOR test.

FIG. 15P shows result of NOR test. Con_Veh, Con_Pic, and VaD_Pic groups, *p<0.05; VaD_Veh, n.s.=not significant. One sample paired t test compared to 50%.

FIG. 15Q shows quantification of c-Fos/NeuN (%) in anterior cingulate (AC). Con_Veh vs VaD_Veh, *p<0.05; Con_Veh vs VaD_Pic, n.s.=not significant. Two-way repeated measures ANOVA.

FIG. 15R shows quantification of c-Fos+ cell numbers (per mm³ because quantification of NeuN+ cells is not accurate due to high density) in hippocampal CA2/3 subregion. Con_Veh vs VaD_Veh, *p<0.05; Con_Veh vs VaD_Pic, n.s.=not significant. Two-way repeated measures ANOVA.

FIG. 15S shows schematic shows the paradigm of piclidenoson treatment starts from 5-days after VaD induction, and the examination of motor function and the change of subtypes of neurons using immunostaining 3-weeks after treatment.

FIG. 15T shows the results of the grid walk task. Three weeks post piclidenoson treatment, Con_Veh vs VaD_Veh, ***p<0.001; VaD_Veh vs VaD_Pic, ##p<0.01; Con_Veh vs Con_Pic, and Con_Veh vs VaD_Pic, n.s.=not significant. 8 animals per group.

FIG. 15U shows quantification of Satb2+/NeuN (%) in Layer VI near infarct lesion. Con_Veh vs VaD_Veh and VaD_Veh vs VaD_CD101, *p<0.05; Con_Veh vs Con_Pic and Con_Veh vs VaD_Pic, n.s.=not significant. Two-way repeated measures ANOVA.

FIG. 15V shows quantification of Cux1+/NeuN (%) in Layer VI above infarct lesion. Con_Veh vs VaD_Veh, **p<0.01; Con_Veh vs VaD_Pic, n.s.=not significant. Two-way repeated measures ANOVA.

FIG. 16A shows CD39-A3AR intercellular signaling related changes in VaD. The expression (FPKM) of Entpd1 (CD39) in 5-cell types in control and VaD mouse brains.

FIG. 16B shows The expression (FPKM) of Adora3 (A3AR) in 5-cell types in control and VaD mouse brains.

FIG. 16C shows the expression (counts perM read) of Entpd1 (CD39) in microglia of normal brain periventricular WM, VaD core and adjacent WM tissue of human patients.

FIG. 16D shows the expression (counts perM read) of Entpd1 (CD39) in EC of normal brain periventricular WM, VaD core and adjacent WM tissue of human patients.

FIG. 16E shows the expression (FPKM) of Adoral (A1AR) in 5 cell types in control and VaD mouse brains.

FIG. 16F shows the expression (FPKM) of Nt5e (CD73) in 5 cell types in control and VaD mouse brains.

FIG. 16G shows the expression (FPKM) of Ada (adenosine deaminase) in 5 cell types in control and VaD mouse brains.

FIG. 16H shows representative images of Glut1 IHC in normal and VaD human brains, which indicate the change of vessel density in VaD.

FIG. 16I shows quantification of Glut1 immunoreactivity by positive pixel per ROI. Con vs VaD, *p<0.05, t test.

FIG. 16J shows representative images of Iba1 IHC showing microglia in normal and VaD human brains.

FIG. 16K shows quantification of Iba1 immunoreactivity by positive pixel per ROI. Con vs VaD, n.s.=not significant.

FIG. 16L shows immunostaining images show the expression of A3AR in microglia (Iba1) in mouse cerebral WM. Asterisk indicates the core of VaD lesion; Scale bar=10 μm.

FIG. 16M shows RNAScope images showing the Adora3 expression in microglia (Aif1) cells in control and VaD WM. DAPI; Scale bar=10 μm.

FIG. 16N shows quantification of Adora3 dots per microglia cell (Aif1+) in (M) in control and VaD mouse brains. Con vs VaD, ***p<0.001, t test.

FIG. 17A shows activation of CD39-A3AR signaling shows neuroprotective and neural repair effects in VaD. Immunostaining show the labeling of cells using AAV.MG1.2-CBh-Flex-Sm-V5 virus in microglia-Cre transgenic strain P2ry12-CreER mice in VaD. Iba1; V5; DAPI. White dashed lines show the boundary of VaD lesion core. Scale bar=100 μm.

FIG. 17B shows zoom-in image shows the specific labeling of microglia in P2ry12-CreER mice in VaD. Iba1; V5; DAPI. Scale bar=50 μm.

FIG. 17C shows single stack confocal images show the colocalization of HA in microglia in P2ry12-CreER mouse brain injected with AAV.MG1.2-CBh-Flex-Entpd1-HA in the VaD lesion core. Arrow heads highlight the colocalization. Scale bar=50 μm.

FIG. 17D shows quantification results of immunoreactivity (% of WM volume in ROI) show the effect of control virus expressing (V5) and Entpd1 (CD39) overexpression (HA) virus expression.

FIG. 17E shows schematic shows the piclidenoson treatment 30-min after VaD induction, and the examination of lesion size and BBB leakage using immunostaining 1˜2-weeks after treatment.

FIG. 17F shows quantification of VaD lesion size in VaD brains treated with vehicle or piclidenoson (Pic).

FIG. 17G shows quantification of BBB leakage by immunoreactivity of mouse IgG. Veh vs Pic, *p<0.05, t test.

FIG. 17H shows immunostaining images show the rescued Satb2 reduction in Layer VI after delayed piclidenoson treatment. NeuN; Satb2; DAPI; Scale bar=10 μm.

### DETAILED DESCRIPTION OF THE INVENTION

Vascular dementia (VaD) constitutes approximately 25% of total dementia. It frequently coexists with Alzheimer's disease (AD) in an additive or synergistic manner; 84% of aged subjects show morphological substrates of VaD in addition to AD pathology. Despite its high prevalence, the precise underlying mechanisms of VaD remain poorly understood, which can be attributed to the lack of suitable preclinical animal models. VaD is characterized by multiple infarcts or ischemia in the periventricular and adjacent white matter (WM), leading to progressive deficits in memory and motor functions. However, the current animal models for VaD predominantly rely on globally induced cerebral hypoperfusion through vessel occlusion in rodents, genetically hypertensive brain and cerebrovascular disease, bilateral/asymmetrical carotid artery stenosis (BCAS/ACAS) or bilateral occlusion of the common carotid arteries (BCAO) in rat or mouse brains. These models suffer from several significant limitations, including in some cases high lethality, substantial variability in lesion size, and the presence of widespread neuronal death in gray matter, which is not observed in human VaD. There is an urgent and significant need for a replicable, robust, and more specific WM-focused mouse VaD model that encompasses the diverse etiologies observed in VaD patients. The cells of the cerebral WM exist in a neurovascular niche, which supports cell homeostasis and injury response through cell-cell signaling. The neurovascular niche has membrane, soluble and extracellular matrix signals that communicate among its constituent endothelial cells (ECs), pericytes, astrocytes and oligodendrocyte progenitor cells (OPCs) in many brain diseases to dictate survival, recovery or further disease progression. The full cell-cell signaling systems in VaD are not known. This VaD cell-to-cell “interactome” may provide starting points for candidate therapeutic systems in this disease.

The present disclosure describes a VaD model using the common C57BL/6J (C57) mouse strain from a WM stroke model using immunocompromised mice. This mouse model reproduces the cellular, circuit, and behavioral impairments in human VaD. Cell type-specific RNA-Seq reveals activation of WM-associated aging genes in VaD mouse model and identifies a unique transcriptome of WM as compared to the same cell types in gray matter (cortex). Intercellular signaling pathways dysregulated in the VaD neurovascular niche were identified from a custom ligand-receptor database. The potential candidates in the same cell types were compared between mouse and human VaD to identify key signaling systems for functional studies. An important extracellular matrix component, Serpine2, and its receptor Lrp1 were identified as elevated during VaD. Knocking down Serpine2 enhanced OPC differentiation towards myelinated oligodendrocytes in VaD. Another key gene system, identified as EC/microglia-microglia signaling through CD39-A3AR, was synergistically downregulated in the conjunction of VaD and aging in human and mouse. An A3AR specific agonist, currently in phase III trials for psoriasis, promoted tissue repair and behavioral recovery in the mouse VaD model in a delayed treatment. The findings of the present disclosure shed light on the intercellular signaling pathways that could serve as therapeutic targets for VaD.

The VaD mouse model induced by WM ischemia faithfully reproduces human VaD's complex pathophysiology, featuring consistent lesions, in the most common location. It mirrors human VaD, exhibiting neural circuit damage and cognitive deficits. Akin to multiple sclerosis, where demyelination leads to decreased expression of specific neuronal transcription factors or subtypes, the VaD model described herein also shows specific reduction of Satb2 and Cux1 in neurons near lesions. While offering advantages, the model does not incorporate or mimic related genetic diseases, blood-brain barrier dysfunction, hippocampal cell death, small hemorrhages, and vascular amyloid deposition.

The present disclosure using TRAP brings new evidence which underscores a unique WM's transcriptomic profile compared to gray matter in a cell type specific manner. The study described herein also fills the gap of a comprehensive WM transcriptomic analysis in normal and VaD brains. Moreover, additional WM-specific cell type markers and transcription factors were identified when compared to cell-specific cortical tissue and whole brain datasets.

For translational relevance, it is preferred to identify intercellular molecular signaling systems that are differentially regulated in both mouse and human VaD: a VaD “interactome”. In the present disclosure, target screening criteria include whether the gene is significantly regulated in both human and mouse RNA-Seq, whether the change is specific to one or two particular cell type(s), and whether the known function is implicated in CNS. Several the highest value (Tier 1) L-R candidates were studied the VaD mouse model described herein. The present disclosure highlights the role of Serpine2-Lrp1 in OPC differentiation, and the ability of downregulating Serpine2 to enhance tissue repair (remyelination) and memory recovery in VaD. Notably, the expression of CD39 remains unaffected solely by the aging process but is influenced by the combined effects of aging and VaD. A3AR is the functional receptor of CD39, and it exhibits specific expression in microglia and experiences a significant reduction in VaD. Notably, the adult brain exhibits limited restorative capabilities, particularly in WM. Initially, infarct lesions in WM are often asymptomatic, yet progress into adjacent areas, resulting in more severe impairments. Delayed treatment with an A3AR agonist significantly enhanced tissue repair and functional recovery. This delayed delivery holds clinical relevance to the disease presentation. Piclidenoson, ((1-deoxy-1-[6-[(iodophenyl)methyl]amino]9H-purine-9-yl]-N-methyl-(-D-ribofuranuronamide), CF101, IB-MECA), a highly selective A3AR agonist undergoing phase III clinical trials for psoriasis, exhibits minimal non-specific effects on other adenosine receptors.

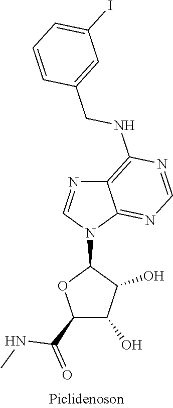 [SMILES: CNC(=O)[C@H]1O[C@@H](n2cnc3c(NCc4cccc(I)c4)ncnc32)[C@H](O)[C@@H]1O]

The approaches in the present disclosure were a discovery-based progression from large-scale molecular expression analysis in human and mouse VaD. The present disclosure represents the first demonstration of a pharmacological approach capable of enhancing tissue repair in the VaD brain.

### Methods

### Animals

All experiments were performed in accordance with National Institutes of Health (NIH) animal protection guidelines and were approved by University of California, Los Angeles Chancellor's Animal Research Committee. Tie2-cre transgenic mice (B6.Cg-Tg (Tek-cre) 1Y wa/J, JAX:008863) were purchase from the Jackson Laboratories. C57BL/6J (C57) mice were purchase from the Jackson Laboratories (JAX:000664) or Taconic Biosciences. Tbx18-creER transgenic strain was crossed with Rpl22-HA transgenic mice (B6J.129 (Cg)-Rpl22tm1.1Psam/SjJ, Jackson Laboratory, JAX:029977) to obtain the Tbx18 (T-box transcription factor 18) reporter mouse line Tbx18-creER::Rpl22-HA animals. Ng2-creER transgenic strain was crossed with Rpl22-HA transgenic mice (JAX:029977) to obtain the OPC reporter mouse line Ng2-creER::Rpl22-HA animals. Serpine2 KO mice were obtained from Dr. Ye Zhang at UCLA. P2ry12-creER (B6(129S6)-P2ry12^{em1(icre/ERT2)Tda}/J, JAX:034727) and Tmem119-2A-CreER (C57BL/6-Tmem119^{em1(cre/ERT2)Gfng}/J, JAX:031820) transgenic mice were purchased from the Jackson Laboratories.

### Virus Constructs

AAV1-CAG-FLEX-EGFP-WPRE (Addgene 51502, >7×1012 vg/mL) and retroAAV-PKG-cre (Addgene 24593, 1.7×1013/μL) were used for PFC-HP long projection study. PHP-CAG-FLEX-Rpl22-HA (4.11×10¹⁴ vg/mL) was produced by packing plasmid pAAV-CAG-FLEXon-Rpl22-3HA in to PHP.eB. Lenti-GfaABC1D-Rpl22-HA was produced by packing plasmid pZac2.1-GfaABC1D-Rpl22-HA (Addgene, 111811) into Lentivirus.

AAV1-CAG-FLEX-EGFP-WPRE (Addgene 51502, >7×10¹² GC/mL) and retroAAV-PKG-cre (Addgene 24593, 1.7×10¹³ GC/mL) were used for PFC-HP long projection study. PHP-CAG-FLEX-Rpl22-HA (4.11×10¹⁴ GC/mL) was produced by packing plasmid pAAV-CAG-FLEXon-Rpl22-3HA in to PHP.eB. Lenti-GfaABC1D-Rpl22-HA was produced by packing plasmid pZac2.1-GfaABC1D-Rpl22-HA (Addgene, 111811) into Lentivirus. AAV.MG1.2-CBh-FLEX-lck-smV5 (3.49×10¹¹ GC/mL) was produced by VectorBuilder using AAV-CAG-flex-lck-smV5 plasmid (Addgene, 196423) and AAV-MG1.2 vector (Addgene, 184541). AAV.MG1.2-CBh-FLEX-Entpd1-HA (9.27×10¹¹ GC/mL) was produced by VectorBuilder using AAV-MG1.2 vector (Addgene, 184541) and custom-made sequence CBh-FLEX-mEntpd1[NM_001304721.1]3×HA.

### Intracranial and Retro-Orbital Virus Injections

For intracranial virus injection, mice were anesthetized with 2% isoflurane and placed in a stereotaxic head frame on a heat pad. Artificial tears were applied to the eyes to prevent eye drying. A midline incision was made down the scalp, and a craniotomy was performed with a dental drill. A Nanoliter injector (World Precision Instruments) was used to infuse virus with Micro4 Controller (World Precision Instruments). Virus was infused at 50-100 nL/min. For PFC-HP long projection study, 10-fold dilution of retroAAV-PKG-cre and 3-fold dilution of AAV1-CAG-flex-EGFP were applied 7-days post VaD. The coordinates for 2 injections (each of 0.5 μL of virus solution) in HP were: anterior-posterior (AP) −2.0 mm, medio-lateral (ML) +1.7 mm, dorsoventral (DV) −1.3 mm (CA1) and 1.6 mm (DG); the coordinates for 1 injection (0.3 μL of virus solution) in medial PFC was: AP +1.70 mm, ML +0.45 mm, DV −1.25 mm. For astrocyte TRAP cell labeling, three injections (each of 0.5 μL of Lenti-GfaABC1D-Rpl22-HA virus solution) were made in the following coordinates: AP +1.45 mm, ML +2.83 mm, DV −1.58 mm; AP +0.15 mm, ML +2.33 mm, DV −1.58 mm; and AP +0.15 mm, ML +3.33 mm, DV −1.60 mm. After infusion, the capillary was kept at the injection site for 5 min before being slowly withdrawn. The incision was closed using VetBond (3M, No. 1469SB). The mice were recovered on a 37° C. heated blanket, and returned to home cage after they woke up. Water with amoxicillin was applied for 1 week.

For retro-orbital virus injection, the protocol was adopted from Yardeni, et al. Briefly, mice were anesthetized with 2% isoflurane through a funnel-shaped nose cone and placed on a 37° C. heated blanket. A 1 mL insulin syringe with 27.5-gauge needle was prepared with 50 μL of PHP-CAG-FLEX-Rpl22-HA virus. The needle was placed so the bevel faces down to decrease the likelihood of damaging the eyeball. The needle was inserted follow the edge of the eyeball down until the needle tip is at the base of the eye. The virus was slowly injected. After the injection was complete, the needle was slowly withdrawn to prevent leak. Mice with obvious bleeding or injectate leakage were not used for further studies. Artificial tears were applied to protect the eyes after injection. The mice were recovered on a 37° C. heated blanket and returned to home cage after they woke up.

### Tamoxifen Preparation and Administration

100 mg of tamoxifen (Sigma T5648) was dissolved in 4.5 mL of prewarmed corn oil (Sigma C8267) at 50° C. The solution was vortexed and warmed repetitively until the tamoxifen was completely dissolved. The final solution was aliquoted and stored in −20° C. until injection day. For Tbx18-creER::Rpl22-HA and Ng2-creER::Rpl22-HA transgenic strains, the mice were injected intraperitoneally at the dosage of 75 mg/Kg/day for 5 consecutive days starting 7 days prior to VaD induction.

### VaD Mouse Model

VaD in the mouse was modified from white matter stroke model. Briefly, the mice were anesthetized with 2% isoflurane and securely mounted onto a stereotaxic apparatus. Core body temperature of the mice was maintained at 36.5 to 37.5° C. A midline incision was made down the scalp, and a craniotomy was performed with a dental drill. A Nanoliter injector (World Precision Instruments) was used to infuse 27 μg/μL of L-NIO (Sigma-Aldrich, 400-600) in sterile saline (Hospira) with Micro4 Controller injector (World Precision Instruments), at the speed of 100 nL/min. To avoid damage to motor cortex, the glass pipette (Wrld Precision Instruments, 1B100F-4) containing the L-NIO was inserted through the cortex of the frontal lobe into the underlying subcortical WM at an angle of 36°. Three injections (each of 0.3 μL of L-NIO solution) were made in the following coordinates: AP +0.80 mm, ML +2.00 mm, DV −1.56 mm; AP +0.80 mm, ML +2.83 mm, DV −1.60 mm; and AP +0.80 mm, ML +3.66 mm, DV −1.61 mm. Localized vasoconstriction leads to focal ischemia in the subcortical WM. Female mice were excluded because estrogen is potentially preventative against ischemic stroke.

### Magnetic Resonance Imaging

Mice were anesthetized and placed in a Bruker 7T small animal MRI (Bruker BioSpin). MRI imaging was performed on 1-2 month after stroke. Respiratory rate was monitored throughout the procedure, and body temperature was maintained at 37°±0.5° C. T2-weighted images were acquired (rapid acquisition relaxation enhancement factor of 8, repetition time of 5300 ms, and echo time of 15.00 ms with an in-plane resolution of 0.0156 mm by 0.0156 mm by 0.50 mm with 13 contiguous slices).

### Brain Tissue Microdissection and TRAP

Mice were anesthetized with isoflurane and perfused with 20 mL cold PBS to remove the circulating macrophages. Coronal forebrain sections (300 μm thick) were collected using mouse coronal section block on cold PBS and placed to a glass dissection surface under a stereoscope maintained at 4° C. Subcortical WM was microdissected from control and VaD brains using fine tipped forceps. Individual samples were transferred into an RNA low-binding 1.5 mL tube and snap-froze on smashed dry ice. All the samples were stored in −80° C. before TRAP procedure. The TRAP protocol was modified from Heiman et al. RNA low-binding tubes with microdissected subcotical WM tissue, one animal per tube, were placed on ice. 300 μL of homogenate buffer was added into each tube. The tissue samples were homogenated using grinding pestles, followed by 30 repetitive pipetting using 200 μL tips. 25 μL of the homogenate was saved as input samples. The rest of the homogenate was centrifuged at 4° C. and 10K rpm for 10 min. The supernatant was transferred to another RNA low-binding tube and incubated with 3 μL of HA antibody (Covance, MMS-101P) with rotation at 4° C. for 4 h. Protein G-magnetic Dynabeads (Thermo Fisher Scientific, 10004D) were washed with homogenizing buffer once and then incubated with homogenate-HA antibody mixture at 4° C. over-night on rotator. HA tagged ribosomes and their associated mRNA were bound to the Protein G magnetic beads at this point. Then, these tubes were placed in magnetic rack to isolate the magnetic beads. The supernatant was removed and the beads were washed with 400 μL/tube of high salt buffer, 3 times at 4° C. on rotator. 300 μL of lysis buffer from RNA extraction kit NucleoSpin® RNA XS (Macherey-Nagel) was added into each tube. The tubes were vortexed for 30 seconds and the supernatant (pulldown samples) containing cell type specific ribosome-associated mRNA were stored in −80° C. until RNA extraction.

### Subcortical White Matter Tissue Dissociation and FACS

The microglia FACS protocol was modified from previous report. Briefly, C57BL/6J mice were anesthetized with isoflurane and perfused with 20 mL cold PBS to remove the circulating macrophages. Subcortical WM was microdissected from control and VaD brains using fine tipped forceps and minced using a scalpel under the stereoscope before being transferred Eppendorf tubes containing 1 mL of Hibernate A solution (Brain Bits) stored on ice. Microdissected WM tissues were gently dissociated in Hibernate A solution using sequential trituration with fire-polished glass pipettes with openings of decreasing diameter (final pipette ˜0.4 mm diameter opening). Resulting cell suspensions were spun down, resuspended in 300 μL 1×PBS and filtered through a 40 μm mesh filter. Cells were then washed once with 1×PBS, resuspended in 300 μL 1×PBS and filtered as above. Filtered cells were then incubated for 20 min on ice with the following antibodies: APC conjugated Rat anti-CD11b (1:100, BD Pharmingen), PE-Cy7 conjugated Rat anti-CD45 (1:400, BD Pharmingen), Brilliant Violet-421 conjugated mouse anti-CX3CR1 (1:200, BioLegend). Throughout the experiment, samples were kept at 4° C. on ice.

Samples were sorted using a FACS Aria cell sorter (BD Biosciences). The population of cells containing microglia could be readily identified based on forward scattering (FSC) and side scattering (SSC) properties. A gating strategy based on FSC and SSC width and height was used to select only single cells.

### RNA Extraction and Sequencing

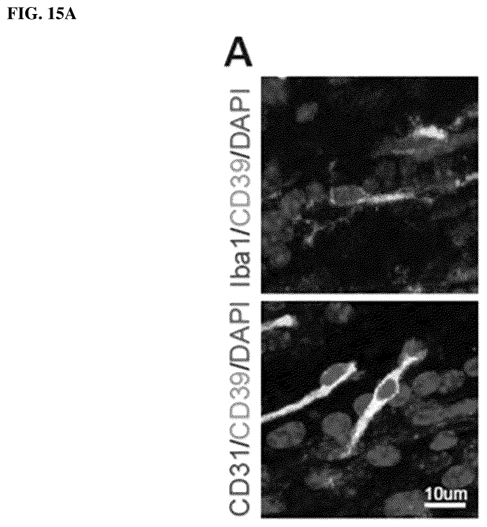

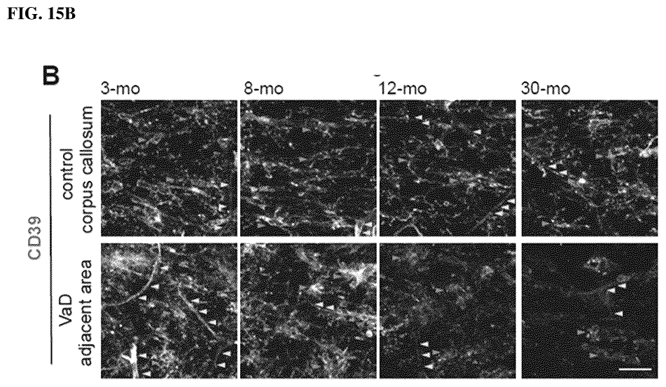

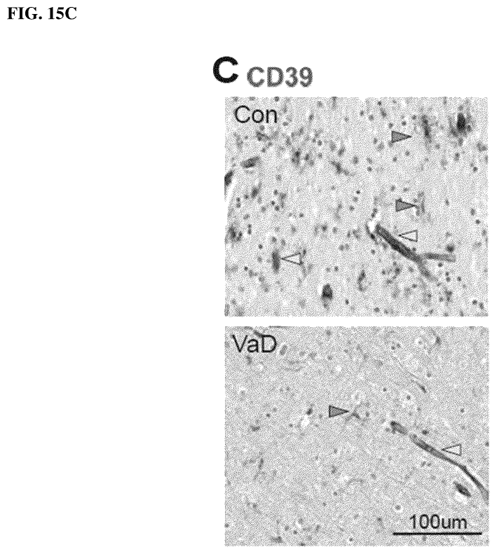

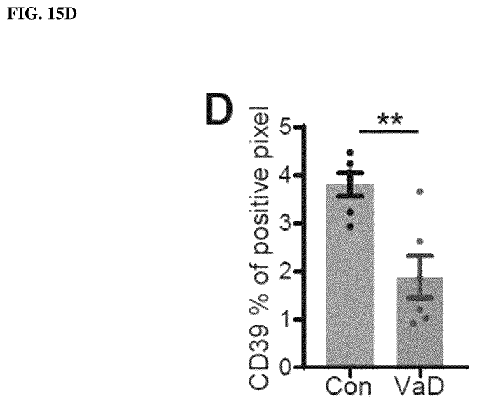

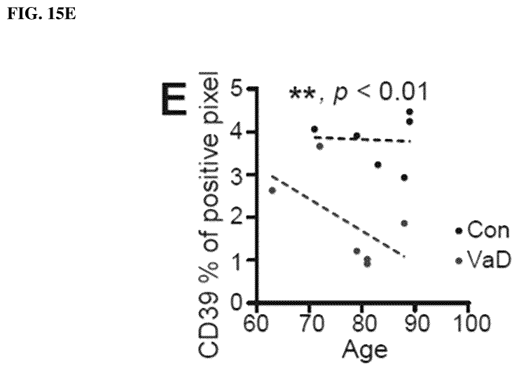

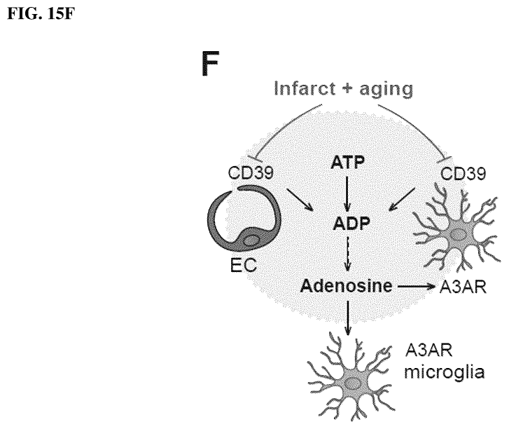

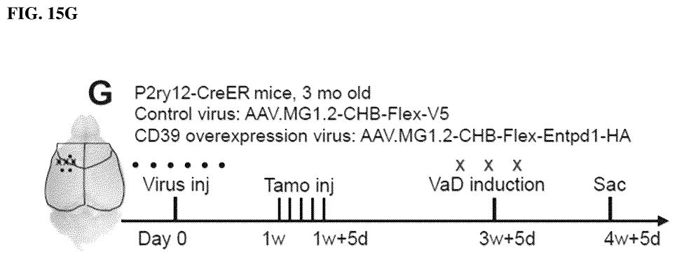

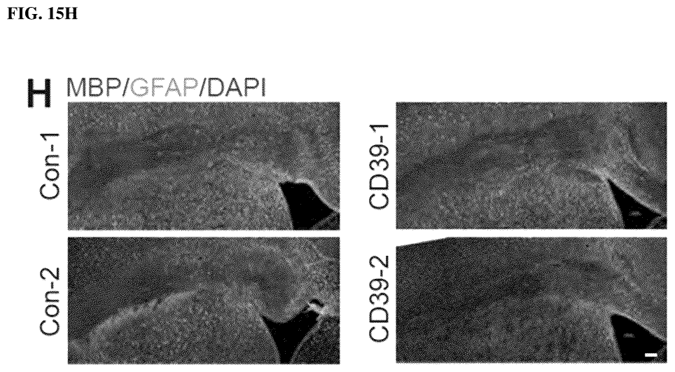

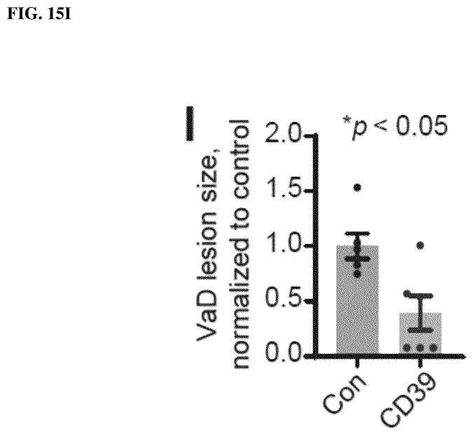

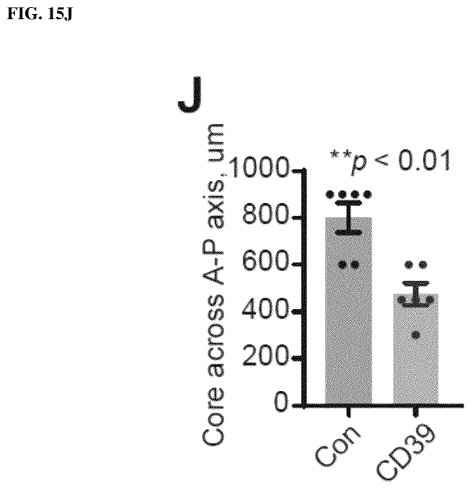

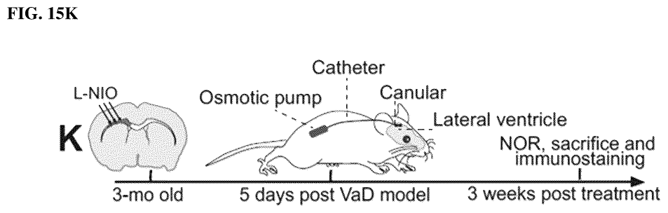

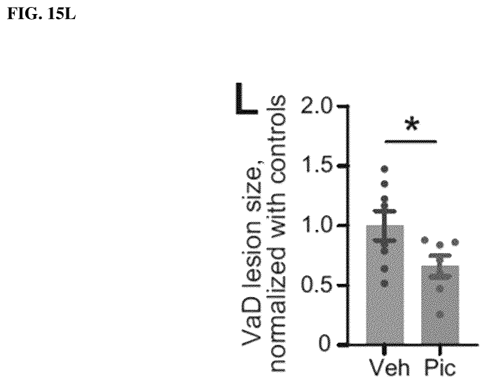

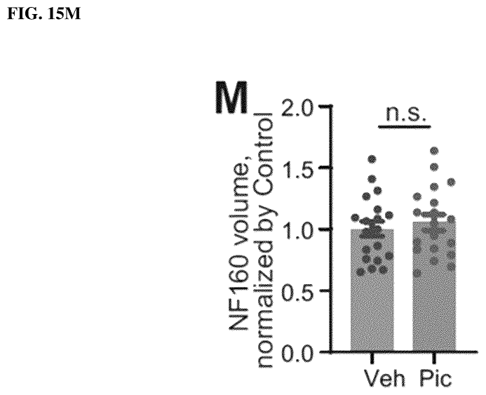

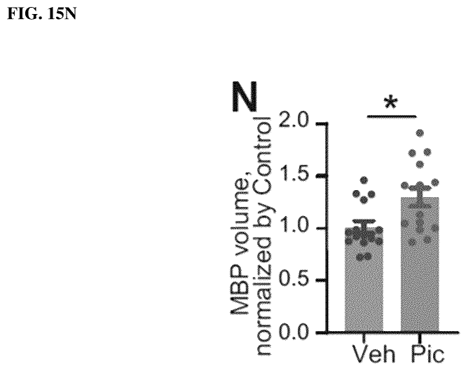

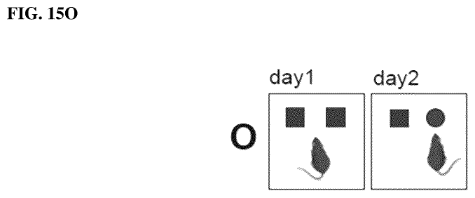

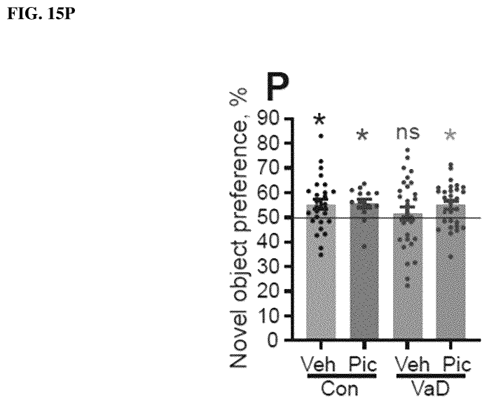

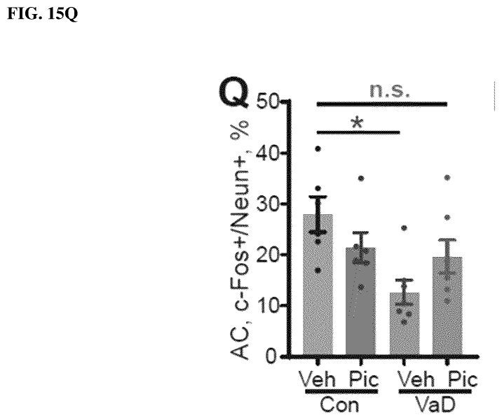

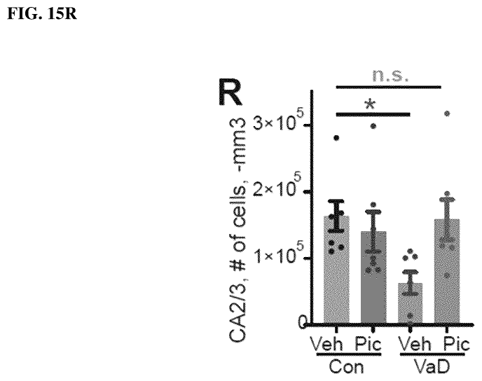

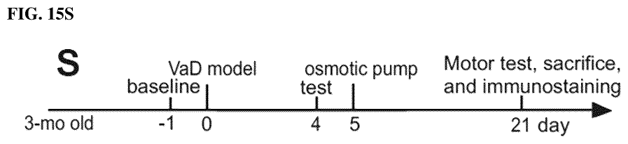

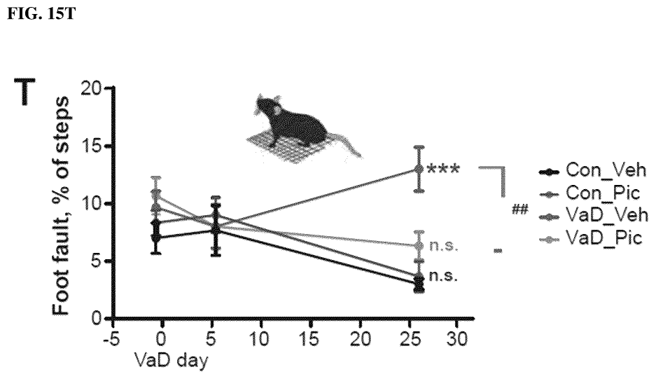

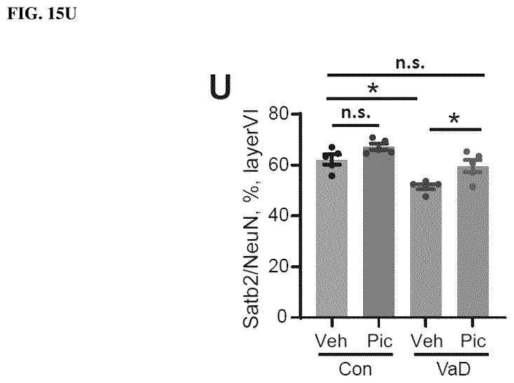

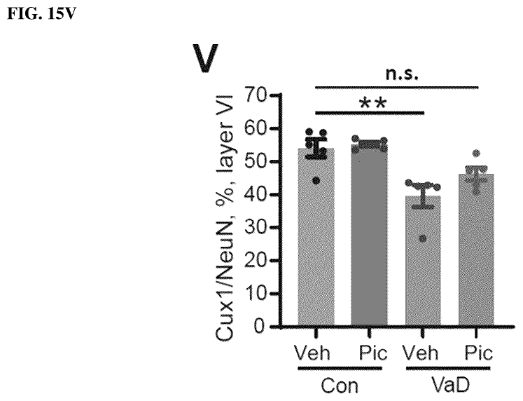

For EC, pericyte, OPC, and astrocyte, total RNA was isolated using the NucleoSpin® RNA XS (Macherey-Nagel), library preparation (Takara SMART-Seq v4 Ultra low Input RNA kit) and sequencing [NovaSeq (PE100/150)] were conducted by MedGenome. Input and pulldown samples were extracted parallelly on the same day. Samples with high RNA integrity number (>6) were used for library construction. More detailed information on the sequencing depth, RIN value and unique alignment rate can be found in FIG. 15. Microglia samples for RIN value test were separate from sequenced samples, with RIN value >6.0. Unique alignment rate for EC, pericytes, OPCs, and astrocytes are between 78-90%. Microglia total RNA was isolated using PicoPure™ kit (Thermo Fisher, KIT0204), library preparation [Nugen Ovation RNA Ultra Low Input (500 pg)+Kapa Hyper] and sequencing (PE 2×75) were conducted by the UCLA Neurosciences Genomics Core (UNGC). The uniquely mapped reads (%) for microglia of Control_P2, VaD_P2, and VaD_P4 groups are 68%-78%; The number of reads per sample are 32-56M. All RNA extractions were stored in RNase-free tubes at −80° C. until further processing.

### Reverse-Transcript PCR, Pre-Amplification, and Quantitative Real-Time PCR

Reverse transcription was performed using PrimeScript™ RT Reagent Kit (Takara, RR037A). Pre-amplification is performed using TaqMan™ PreAmp Master Mix (4391128). Quantitative real-time polymerase chain reaction (qRT-PCR) were performed using Premix Ex Taq™ (Takara, RR390A). All were performed following the vendor provided protocols. The PCR probes used in this study are listed below.

| Gene name | Taqman probe Cat# |
|---|---|
| Aif1 | Mm00479862_g1 |
| Gapdh | Mm99999915_g1 |
| Gfap | Mm01253033_m1 |
| Pdgfrα | Mm00440701_m1 |
| Pdgfrβ | Mm00435553_m1 |
| Tek | Mm00443243_m1 |

### Bioinformatics

To identify DEGs, Hiset2 or STAR were used to align reads to the GRCm38 genome assembly. An FDR value threshold of 0.1 was imposed for the likelihood ratio test to select DEGs, using edgeR, limma, and voom packages. Expression level estimation was reported as fragments per kilobase of transcript Fragments Per Kilobase Million (FPKM) value

To construct custom-made L-R database, the lists of L-R interactions were downloaded from 3 major databases. R package tidyr was used to merge the databases and remove the duplicates. The source database and species (human or mouse) were annotated. R package circlize was used to draw Circos plots.

To identification of WM specific transcriptional factors (TFs) and cell type markers, the mouse TFs were downloaded from KEGG and the canonical cell type markers were adopted from Barres' database. A stringent approach was employed to identify WM and cell type-specific TFs/markers based on, 1) FPKM at least two-fold higher than in other cell types, 2) enrichment fold change >1 (calculated as the fold-change of FPKM, pulldown vs input), and 3) exclusion of genes with an FPKM<10. The calculation of normalized specificity in WM, cortex, or the whole brain involves dividing the FPKM value in the target cell by the average FPKM value across all cell types (including the cell type being considered) and then further dividing this result by the total number of cell types. This computation yields a value within the range of 0 to 1, where 0 signifies no expression, and 1 indicates that the gene is exclusively expressed in the target cell type.

To identify ECM and GPCR genes in different cell types, gene ontology annotations of ECM and GPCR were downloaded from MGI. Bubble plots were drawn based on the cell-type specific expression of ECM and GPCR genes, and their changes in VaD.

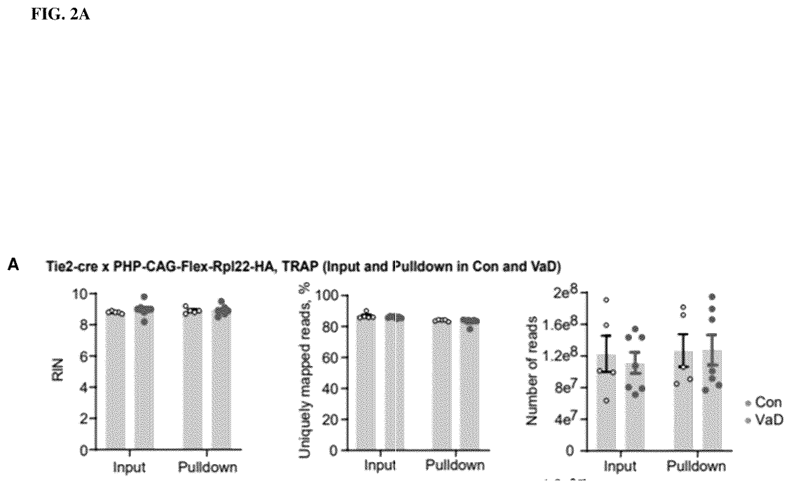

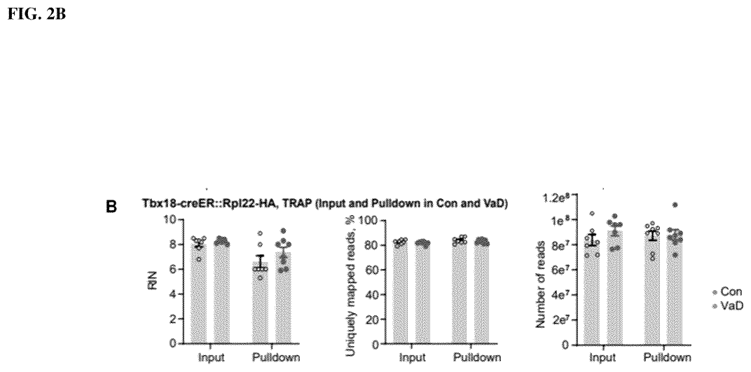

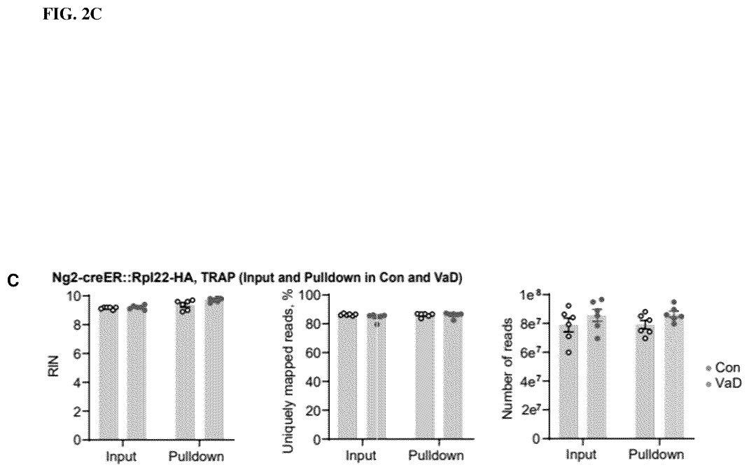

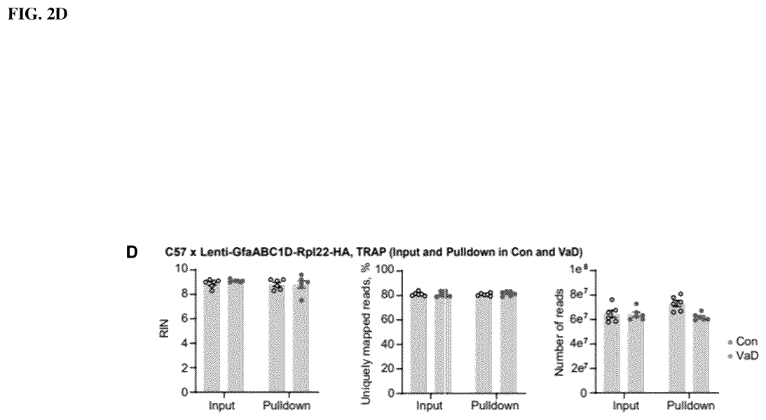

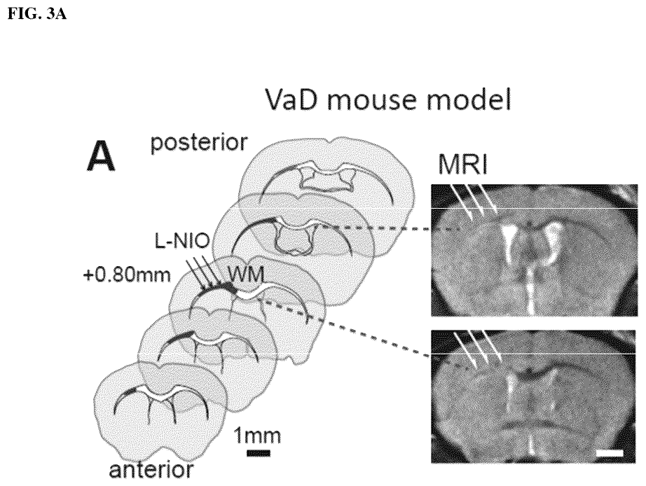

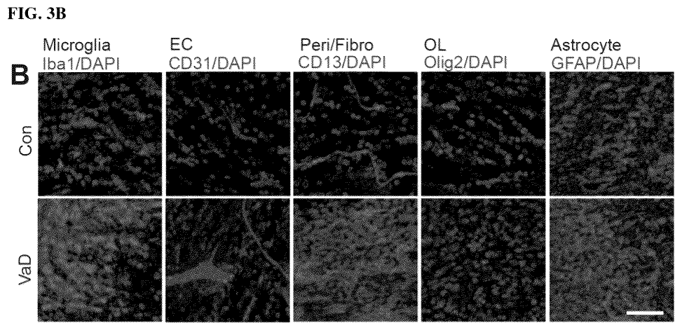

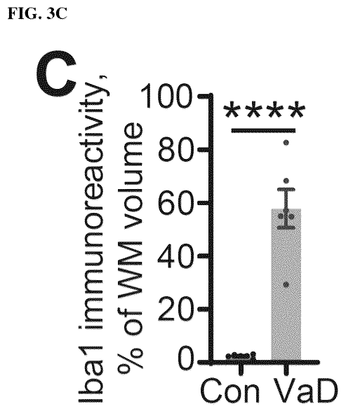

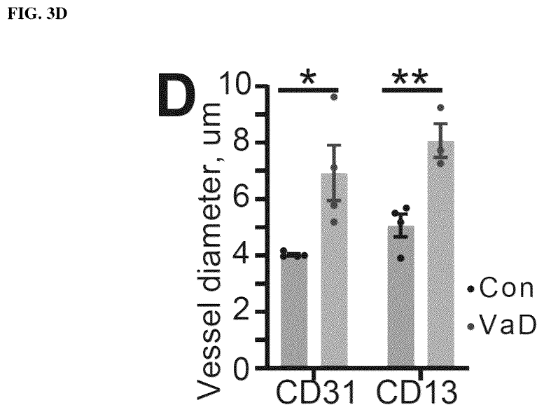

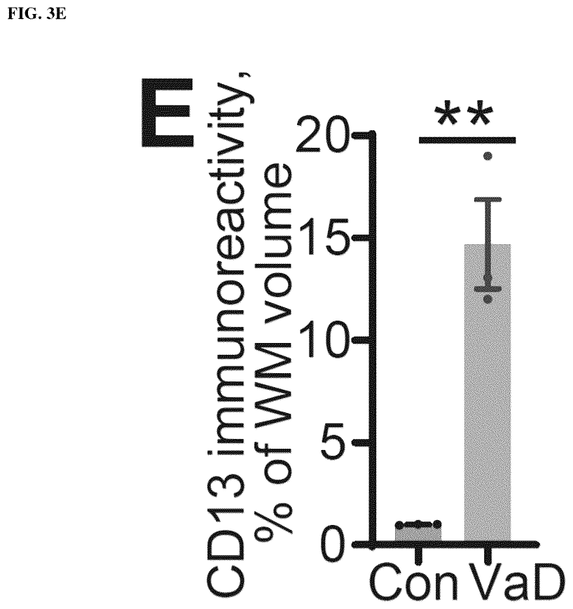

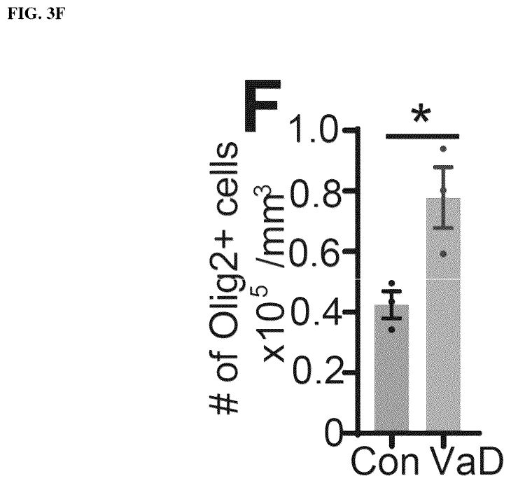

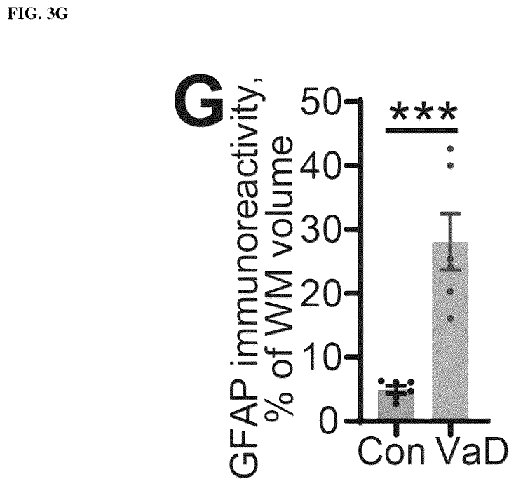

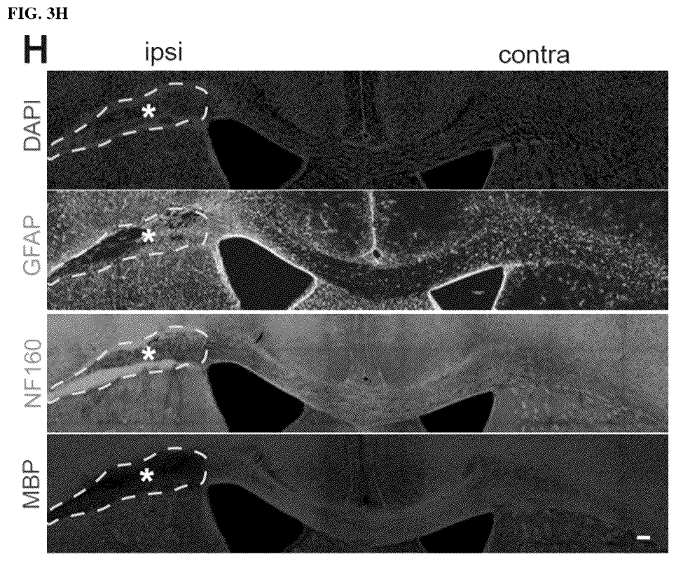

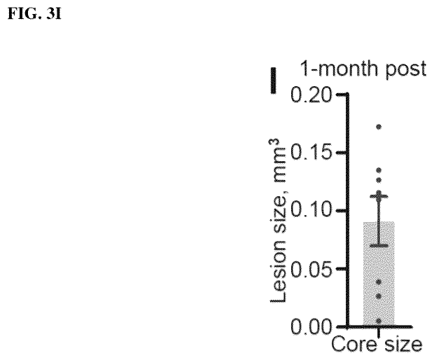

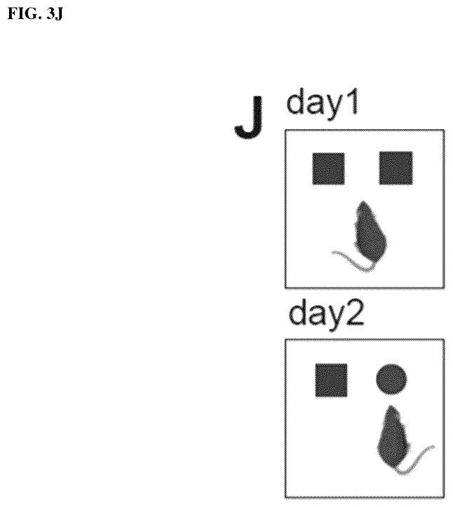

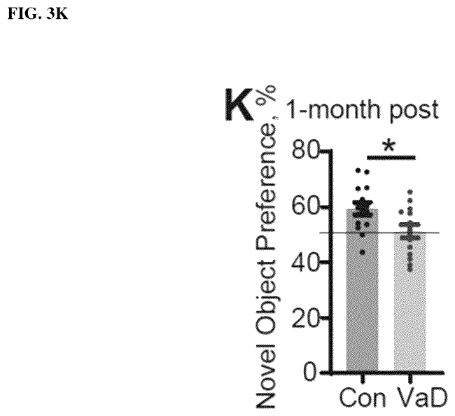

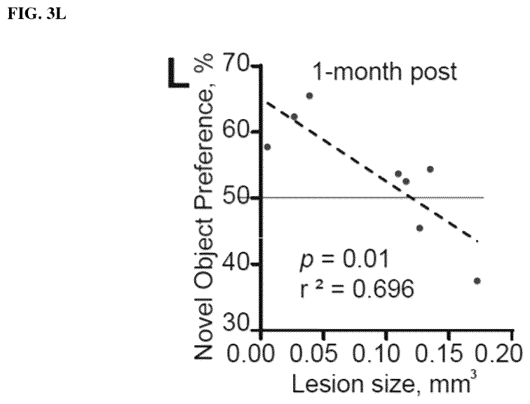

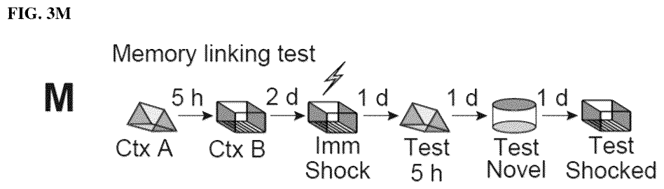

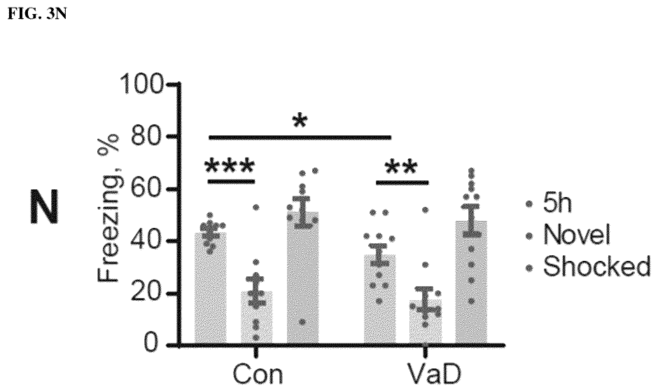

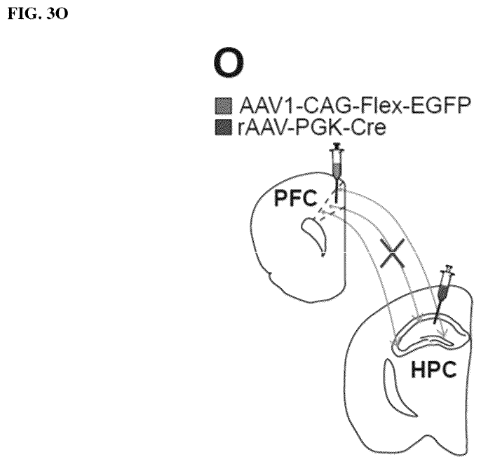

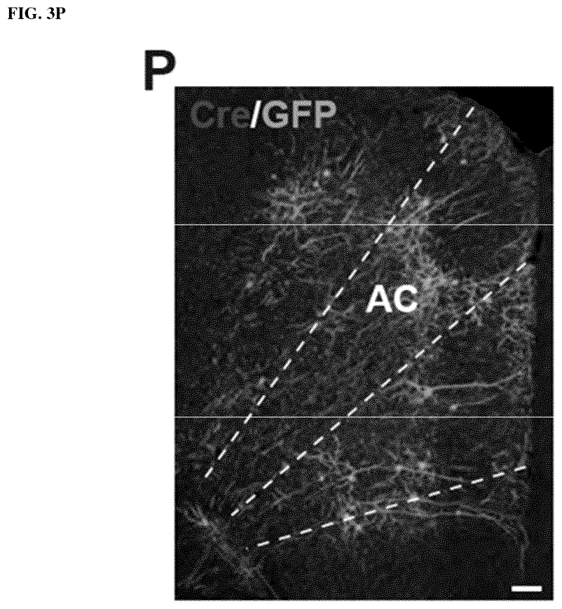

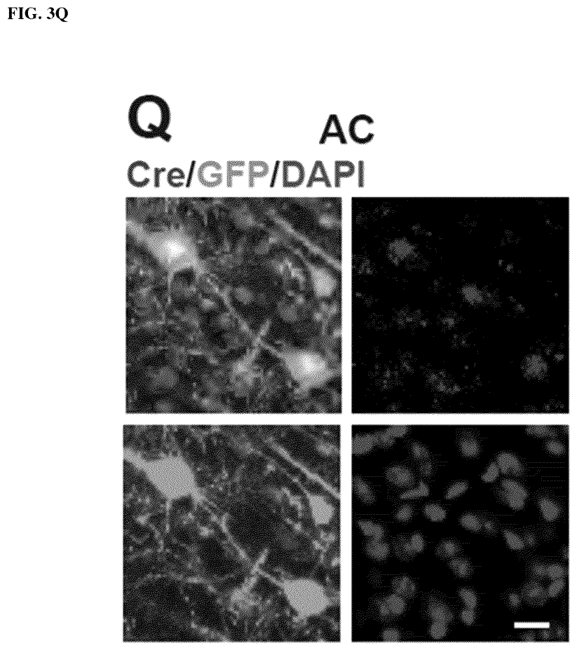

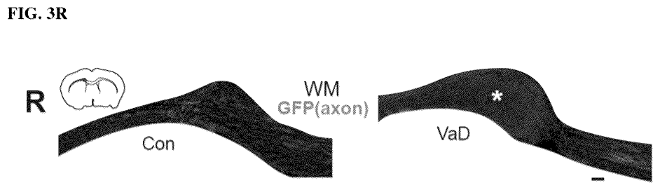

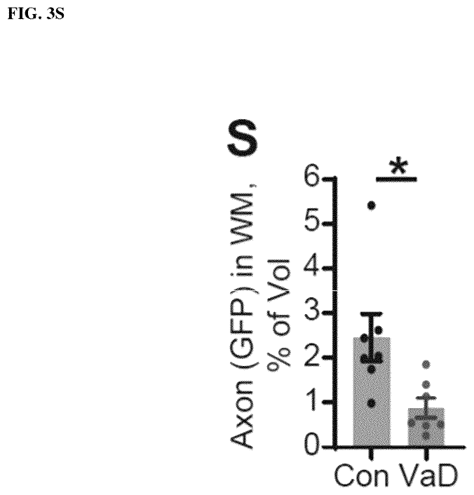

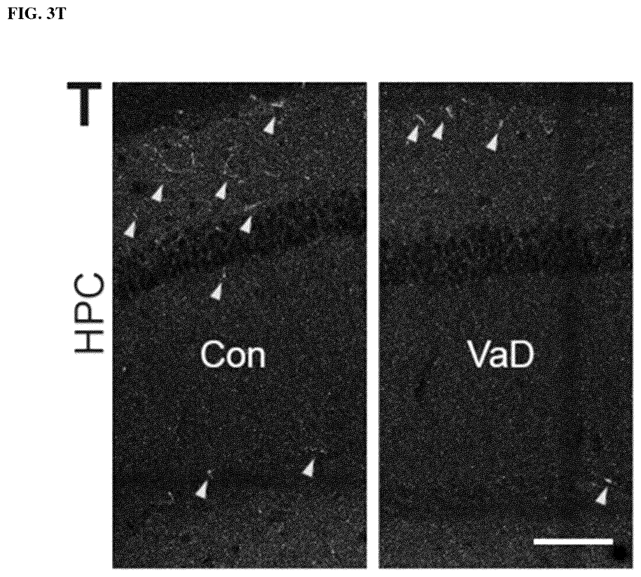

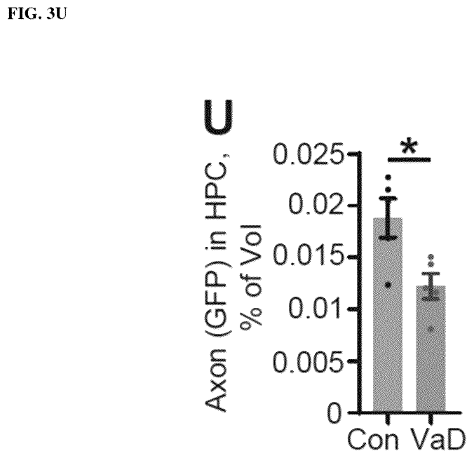

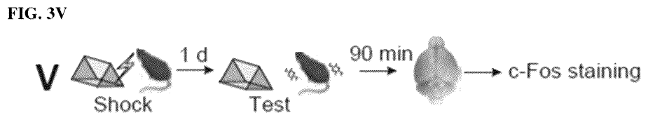

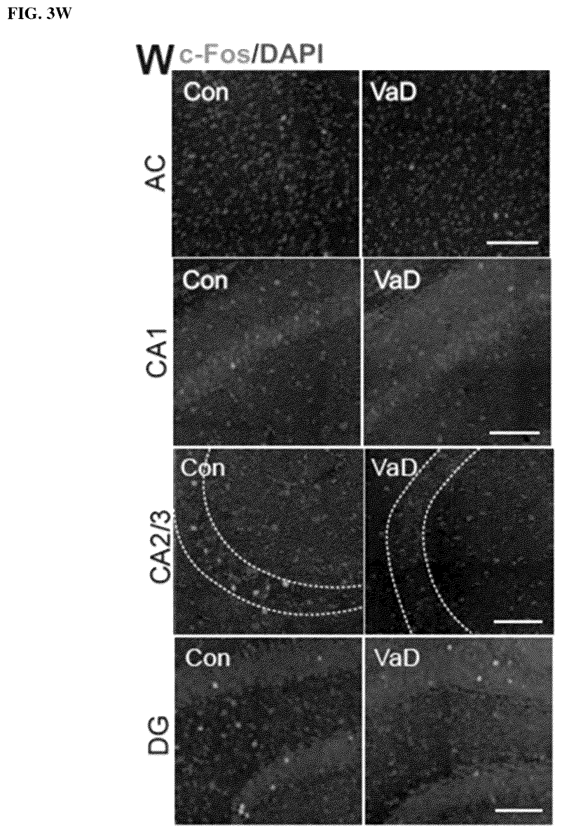

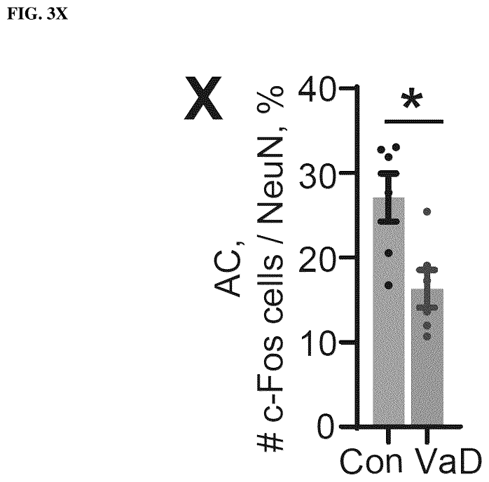

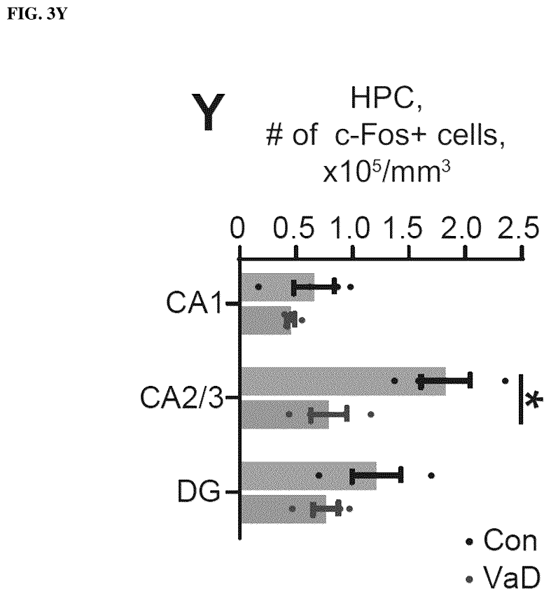

The Venn diagrams in FIGS. 2 and 3 were draw using R package ggvenn
The tiering strategy was based a combination of systematic informatic approach and literature review. According to FIG. 25A, Tier 6 to Tier 3 were screened using pure informatic approach. Tier 3 is readily of a short list so that all the L-R candidates can be simply judged by manual literature exploration—whether it is implicated in CNS function: Yes□Tier 1, No□Tier 2.

Rank-rank hypergeometric overlap (RRHO) analyses were performed using average FPKM from control samples of the WM dataset, compared with Barres' dataset, which was filtered to include only the genes with an FPKM greater than 1 for each cell type. These were also compared with Betsholtz's dataset, which was generated by bulk-normalizing counts to produce FPKM values.

Gene set enrichment analysis (GSEA) was done using GSEA 4.3.2 with MsigDB ver7.0.

KEGG and GO pathway analyses were done by ShinyGO0.77.

### In Situ Hybridization

Mouse brains were dissected and flash-frozen in OCT by dry ice without PFA fixation. 15 μm frozen sections were sliced using cryostat. In situ hybridization was performed using RNAscope Fluorescent Multiplex Reagent Kit V2 (ACD, 323110) according to the manufacturer's instructions. RNAscope Probe-Mm-Adora3-O1 (ACD, 461891) was used to detect Adora3 mRNA. Probe-Mm-Aif1-C2 (ACD, 319141-C2) was used as marker for microglia. Probe-Mm-Anpep-C3 (ACD, 417181-C3) was used as marker for microglia. Probe-Mm-Serpine2-C3 (ACD, 435241-C3) was used as marker for Serpine2 mRNA.

### Immunostaining Using Mouse Brain Sections and Confocal Images

Animals were perfused transcardially with 0.1 M phosphate-buffered saline (PBS), followed by 4% paraformaldehyde (PFA). The brains were removed, post-fixed overnight in 4% PFA, and sectioned into consecutive 50-μm-thick slices using vibratome (Leica).

Immunostaining was performed by brain section blocking in 5% normal donkey serum for 1 hour at room temperature, incubating in primary antibody overnight at 4° C., and in secondary antibody for 1 hour at room temperature. For the performance using CD39 and Pdgfrβ antibodies, antigen retrieval using sodium citrate buffer (10 nM, pH 6.0), in pressure cooker for 3 min was applied before blocking. All sections were counterstained with 4′,6-diamidino-2-phenylindole (DAPI). The antibodies used in this study are listed below.

| Antibody | Vendor/Species/Cat # | Concentration |
|---|---|---|
| A3AR | Abcam/Rabbit/ab197350 | 1:300 |
| Aspa | EMD Millipore/Rabbit/ABN1698 | 1:500 |
| CD13 | Abcam/Rat/ab33489 | 1:300 |
| CD31 | R&D/Rat/AF3628-SP | 1:300 |
| CD31 | Invitrogen/Rabbit/PA5-16301 | 1:300 |
| CD39 | Abcam/Rabbit/ab223842 | 1:500 |
| c-Fos | Cell Signaling/Rabbit/2250 | 1:500 |
| Cre- | Synaptic Systems GmbH/Guinea pig/ | 1:500 |
| Recombinase | 257004 | |
| Ctip2 | Abcam/Rat/ab18465 | 1:500 |
| Cux1 | Proteintech/Rabbit/11733-1-AP | 1:500 |
| Gfap | Invitrogen/rat/13-0300 | 1:2000 |
| Glut1 | Millipore Sigma/Rabbit/07-1401 | 1:500 |
| HA | Roche/Rat/11867431001 | 1:300 |
| V5 | Absolute antibody/Human/Ab00136- | 1:200 |
| | 10.0 | |
| Iba1 | Wako/Rabbit/019-19741 | 1:1000 |
| Iba1 | Abcam/Goat/Ab5076 | 1:500 |
| Lrp1 | Thermo Fisher Scientific/Rabbit/BS- | 1:100 |
| | 2677R | |
| MBP | Abcam/Rabbit/Ab40390-1001 | 1:500 |
| NeuN | Chemicon/Mouse/MAB377 | 1:1000 |
| NeuN | Synaptic Systems GmbH/Guinea pig/ | 1:500 |
| | 266004 | |
| NF160 | Abcam/Mouse/Ab7794-1001 | 1:500 |
| Olig2 | EMD Millipore/Rabbit/AB9610 | 1:500 |
| Pdgfrα | R&D/Goat/AF1062 | 1:500 |
| Pdgfrβ | R&D/Goat/AF1042 | 1:500 |
| Satb2 | Abcam/Rabbit/ab92446 | 1:1000 |
| Anti-Goat-IgG | Jackson ImmunoResearch/Donkey | 1:1000 |
| Anti-Guinea Pig- | Jackson ImmunoResearch/Donkey | 1:1000 |
| IgG | | |
| Anti-Mouse-IgG | Jackson ImmunoResearch/Donkey | 1:1000 |
| Anti-Rabbit-IgG | Jackson ImmunoResearch/Donkey | 1:1000 |
| Anti-Rat-IgG | Jackson ImmunoResearch/Donkey | 1:1000 |
| Anti-Human-IgG | Jackson ImmunoResearch/Donkey | 1:1000 |

High-resolution confocal images in z-stacks were acquired (Nikon C2). Area measurements of the infarct core, WM axonal projections stained with NF160 and MBP were quantified with Imaris (Bitplane, version 10.0.0) using surface function; all the channels were threshold with the same absolute intensity against the background. The parameters for scanning were kept constant across treatment, and conditions.

### Immunohistochemistry (IHC) Using Human Brain Sections and Whole Slide Imaging

IHC protocol was modified from previous reports. Briefly, formalin-fixed periventricular white matter blocks of normal and VaD patients were obtained from the NIH brain bank. Samples were paraffin embedded, sectioned at 7 μm in thickness, immunostained with CD39, Glut1, or Iba1 primary antibodies followed by either horse anti-mouse or horse anti-rabbit secondary antibody conjugated to horseradish peroxidase (HRP), visualized with DAB as chromogen (Vector Laboratories, S-2012), and counterstained with hematoxylin. Slides were then scanned and digitized using the Pannoramic Midi 2 (Epredia). The intensity of immunoreactivity was analyzed using the Positive Pixel Count algorithm in the ImageScope program. The antibodies used in this study are listed below.

| Antibody | Vendor/Species/Cat # | Concentration |
|---|---|---|
| CD39 | Abcam/Rabbit/ab223842 | 1:500 |
| Glut1 | Millipore Sigma/Rabbit/07-1401 | 1:500 |
| Iba1 | Invitrogen/Mouse/MA5-27726 | 1:300 |
| Anti-Mouse-IgG-HRP | Vector Laboratories/Horse/ | N/A |
| | MP-7402-15 | |
| Anti-Rabbit-IgG-HRP | Vector Laboratories/Horse/ | N/A |
| | MP-7401-50 | |

Patient information is listed below.

| NIH brain | | | |
|---|---|---|---|
| bank sample # | Sex | Age | Diagnose |
| 21278 | Female | 84 | VaD/LOAD* |
| 298992 | Female | 86 | No dementia |
| 33987 | Male | 63 | No dementia |
| 17629 | Female | 81 | VaD |
| 17678 | Male | 89 | VaD |
| 53706 | Female | 79 | VaD |
| 36866 | Female | 79 | No dementia |
| 34913 | Male | 85 | No dementia |
| 236440/81764 | Female | 88 | VaD/LOAD* |
| 46146 | Male | 71 | No dementia |
| 9700 | Male | 72 | VaD |
| 73095 | Male | 81 | VaD |
| 54549 | Male | 86 | VaD |
| 63781 | Male | 63 | VaD |
| 236428/56356 | Female | 79 | No dementia |
| 86213 | Male | 89+ | No dementia |
| 11371 | Male | 89+ | No dementia |
| 1176 | Female | 85 | No dementia |
| 17230 | Female | 89+ | No dementia |
| 90996 | Female | 89+ | VaD |
| 51570 | Male | 89+ | VaD |

*LOAD: late onset Alzheimer's disease. LOAD was diagnosed in addition to VaD after samples were request by authors.

### Quantification for Fluorescence and Immunohistochemistry Images (FIG. 3A)

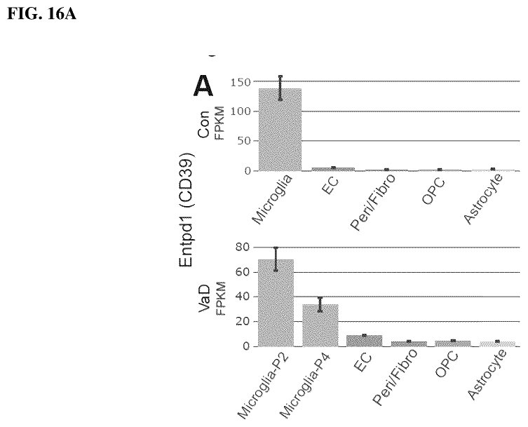

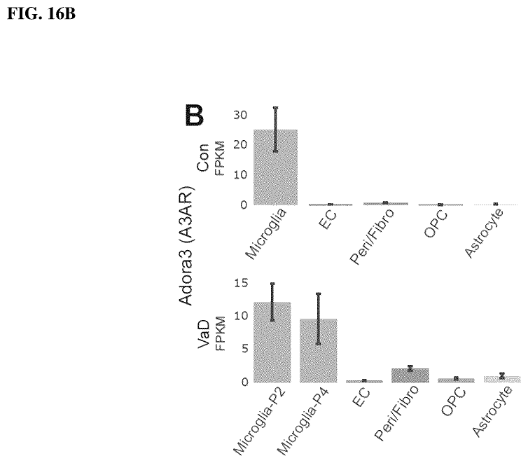

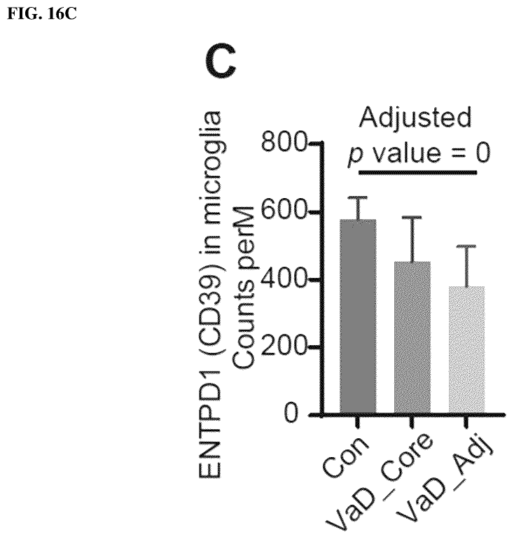

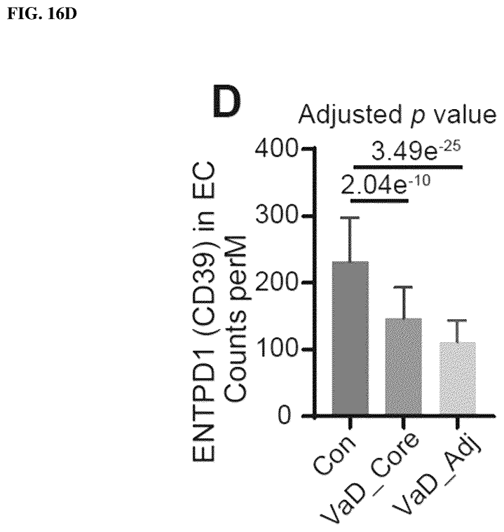

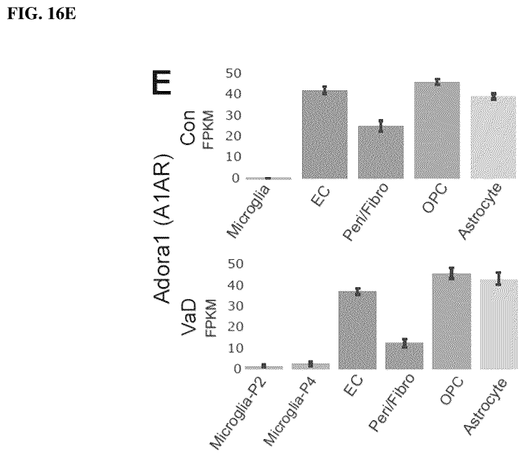

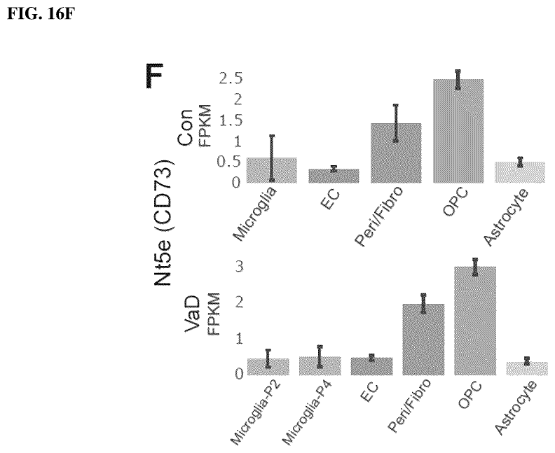

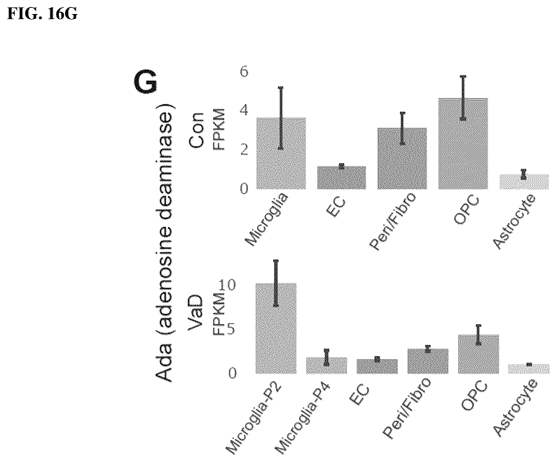

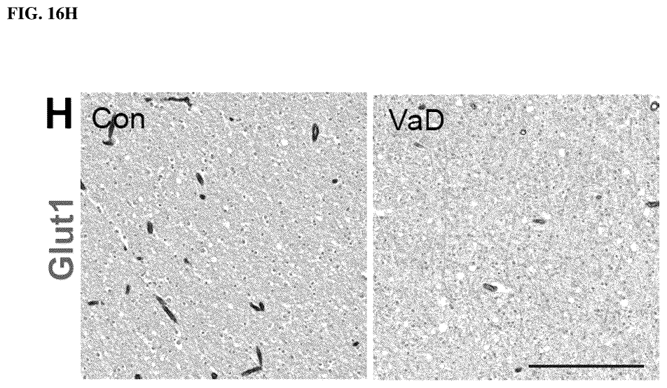

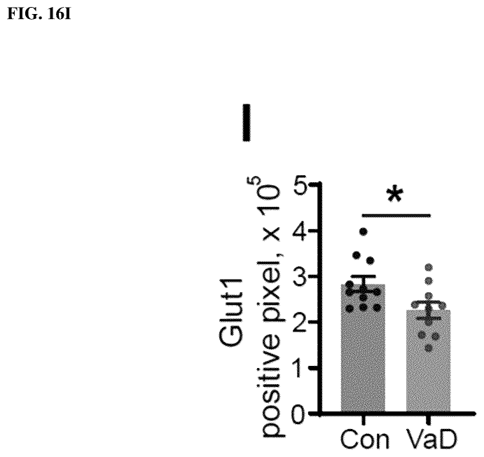

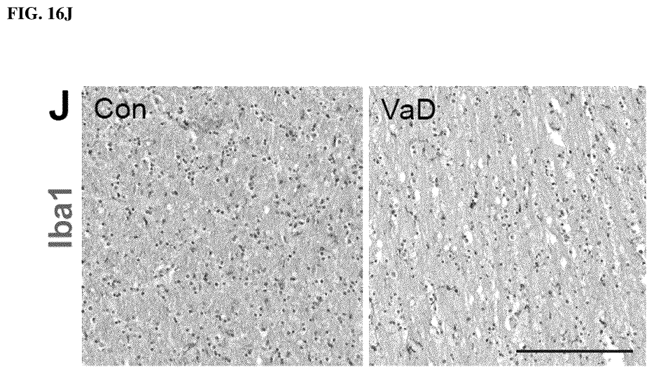

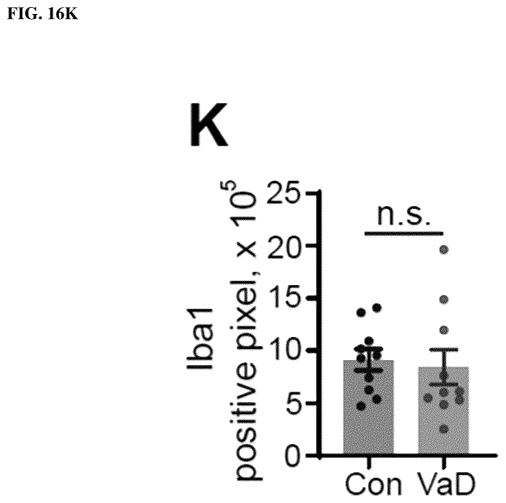

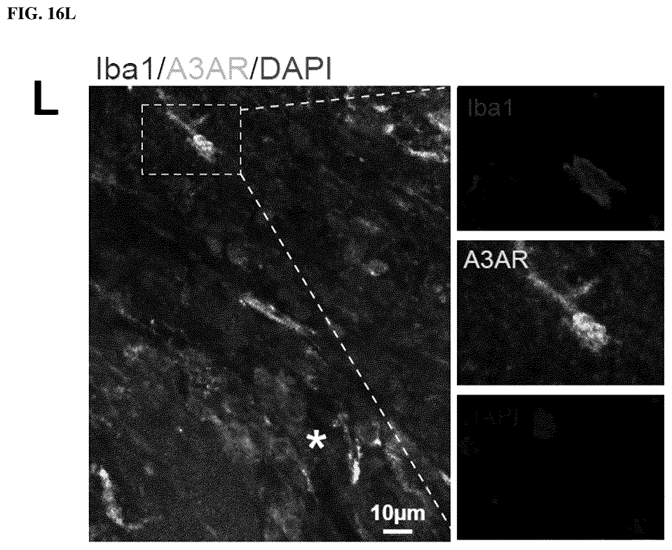

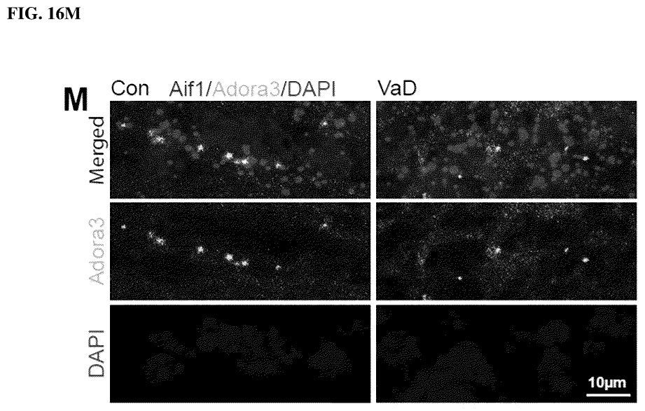

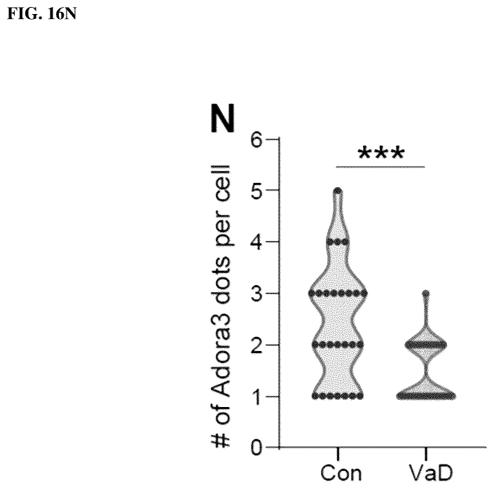

1. Quantification of MBP and NF160 volume for FIGS. 26I, 28M, 28N. Imaris (Bitplane, version 10.0.0) using surface function; all the channels were threshold with the same absolute intensity against the background. Because the absolute volume of cerebral white matter varies between coronal slices depending on the coordinate along anterior-posterior axis, it is difficult to directly compare the absolute volume of NF160 or MBP between groups. The volume of ipsilateral white matter is very similar to the contralateral white matter, we first normalized the volume of NF160 or MBP at the ipsilateral side to the contralateral side (no lesion), and then compare the normalized value between groups. This may be a more reasonable way to investigate how the axon reprojection and myelin regrowth is changed by piclidenoson (CF101) treatment. For each animal, 4 slices, with 300 μm interval across the lesion core along the anterior-posterior axis, were quantified.
2. Quantification of absolute core sizes (FIG. 16F). For each animal, all the slices that cover the lesion core were stained with MBP, NF160, DAPI, and GFAP. The area of core is defined as brain region with NF160/MBP debris, high density of DAPI, and GFAP boundary. The size of core area is measured in ImageJ. The absolute core size is defined by the sum of core area×the slice thickness from all slices.
3. Quantification of relative core sizes (FIG. 28I, 28L). For each animal, 4 slices, with 300 μm interval across the lesion core along the anterior-posterior axis, were quantified using ImageJ. The relative core sizes were normalized by the average of vehicle treated group.
4. Quantification of c-Fos cells (FIG. 16R, 16S, 28Q, 28R). For slices from prefrontal cortex, the number of c-Fos cells were quantified by Imaris using spots function, and normalized with the number of neurons (NeuN+) in the same region of interest (ROI). For slices from hippocampus, the number of c-Fos cells were quantified by Imaris and normalized with the volume of ROI because it is difficult to normalize with the number of neurons due to compact pyramidal cell layer. The same quality threshold was used for all samples.
5. Quantification of CD39 in human IHC (FIG. 28D, 28E). The intensity of immunoreactivity was analyzed using the Positive Pixel Count algorithm in the ImageScope program. The same intensity threshold was used for all samples.
6. Quantification of axon volume in white matter and hippocampus (FIG. 16M, 160). For each animal, slice from the interested region were quantified by surface function in Imaris. The same quality threshold for GFP channel was used for all the samples. The average volume per section was compared between groups.
7. Quantification of Satb2+, Cux1+, NeuN+, Ctip2+, and DAPI+ cells (FIGS. 16U, 16V, 16W, 18E-I, 28U, 28V). In Imaris, surface function was used to define the white matter lesion boundary and the cortical region. The number of cells was quantified using spots function with the same quality threshold for all the samples, and then classified by distance to the white matter lesion boundary, 0-100 μm, 100-200 μm, 200-300 μm, 300-400 μm, 400-500 μm, 500-600 μm, etc. 8. Quantification of ASPA+Olig2+EdU+ triple positive cells (FIG. 26L). In Imaris, surface function was used to define the white matter lesion boundary and the cerebral white matter region. Spots function in Imaris was used for quantification of each cell type with the same quality thresholding. The triple positive cells were defined as ASPA+spots with <5 μm distance to Olig2+ and EdU+spots. The result was further classified by distance to white matter lesion boundary, <0 μm, 0-100 μm, 100-200 μm, 200-300 μm, 300-400 μm, 400-500 μm, 500-600 μm, etc.
9. Quantification of HA+Olig2+ cells (FIG. 20E). The number of Olig2+ and HA+Olig2+ cells were manually counted using ImageJ.
10. Quantification of glial and vascular cell responses in VaD (FIGS. 16C-16G). Imaris (Bitplane, version 10.2.0) was used. The immunoreactivity of Iba1, CD13, and GFAP was quantified using surface function. The same intensity threshold was used for all samples. The volume of positive pixels was normalized with the volume of ROI in WM (%) (FIGS. 16C, 16E, and 16G). The vessel diameter was measured with slide view of CD31 or CD13 channel (FIG. 16D). Five to ten vessels in the same slice were measured and averaged for the value of each animal. The number of oligodendrocyte lineage cells was quantified using spot function and normalized with the volume of ROI in WM (/mm³) (FIG. 16E). The same quality threshold was used for all samples in spot function.
11. Quantification of virus expression (FIG. 30D). Imaris (Bitplane, version 10.2.0) was used. The immunoreactivity of V5 or HA was measured by the volume of positive pixels in corresponding channels using surface function with the same intensity threshold in one channel for all samples. Then, the total volume of positive pixels was normalized with the volume of ROI in WM (%).

### Osmotic Pump and Brain Infusion Kit Implantation

Osmotic mini-pumps (1002, 1004) were subcutaneously implanted and connected to right lateral ventricle through brain infusion kit 3 (Alzet). The brain infusion kits were fixed onto the skull with super glue (Loctite, 45198). For 4-week treatment, 1004 mini-pumps were used to delivery 400 M of piclidenoson (in 1% DMSO in saline) at the speed of 0.11 μL/h; For 2-week treatment, 1002 mini-pumps were used to deliver 200 μM of piclidenoson at the speed of 0.23 μL/h.

### Behavior Tests

C57 mice with or without VaD were tested in Fear Conditioning (FC), Memory association, Novel Object Recognition (NOR), and Grid Walking tasks. All tests were performed 3-4 weeks after VaD. For FC, testing mice were first handling for 3 days (1 min/day) and then habituated to transportation and external environmental cues for 2 min in the experimental room each day for another 3 days. During FC test, mice explored the context for 2 min and then shocked for 2 s (0.65 mA). 58-s after the shock, mice were placed back in their home cage. 1 day later, the mice were returned to the same context for 5 min.

For NOR, the testing mice were allowed to explore an open field arena (30 cm×30 cm) with 2 identical objects for 12 min on the first day. Then the one object was replaced to a novel object and the mice were allowed to explore the 2 different objects for 8 min. The exploration was recorded and the time to explore each object was analyzed by ANY-maze. The data were presented as exploration ratio of time spent exploring the novel object versus both objects.

For NOR, mice were habituated in an open field arena (41.5 cm×41.5 cm×41.5 cm) for 10 min. During the training session, mice were back to the open field and explored with two identical objects for 10 min on the first day. After a 24-hour interval, mice were tested with the previously presented object and a novel object for 10 min. Different sets of objects were used in the 2-month NOR. The exploration was recorded and the time to explore each object was analyzed by ANY-maze, and/or manually hand-scored. The data were presented as the exploration ratio of time spent exploring the novel object versus both objects.

For Grid walking test, the mice were tested 1-day pre-, 7-day, 30-day, and 60-day post-VaD in progressiveness investigation, and 1-day pre-, 4-day and 21-day post-VaD in piclidenoson treatment study. Behavior tests were scored by observers who were masked to the treatment group of the animals.

Memory linking is a process by which new information is linked to previously stored information in the brain. This linking process helps to store information in a structured way and makes it easier to retrieve later. By forming connections between new and old information, memory association enhances the strength of memory traces and helps to organize the information in a meaningful way, leading to more effective memory storage and recall. Compared to single memory, memory linking is more sensitive to changes in the microenvironment (e.g., aging).

Specifically, mice explored 2 different contexts (A and then B, counterbalanced) which were separated by 5 h-7 d. Mice explored each context for 10 min. For immediate shock, mice were placed in chamber B for 10 s followed by a 2-s shock (0.65 mA). 58 seconds after the shock, mice were placed back in their home cage. For the context tests, mice were returned to the designated context (A, B and a novel context C, counterbalanced). The freezing was assessed via an automated scoring system (Med Associates) with 30 frames per second sampling; the mice needed to freeze continuously for at least one second before freezing could be counted.

### Statistical Analysis and Reproducibility

The investigators who collected and analyzed the data including behavior, bioinformatic analysis, mouse brain immunostaining, human brain IHC, were blinded to the VaD and treatment conditions. Mice were randomly allocated to treatment condition using a randomized block experimental design (restricted randomization). All data are demonstrated in figures as means±SEM. All statistical analyses were performed using GraphPad Prism 8. For behavior experiments, RNA-Seq, quantification of immunostaining and human IHC, n designates the number of mice or patients. Statistical significance was assessed by Student's t test, or one- or two-way ANOVA where appropriate, followed by the indicated post hoc tests for repeated measures. Significance levels were set to P=0.05. Significance for comparisons: *P<0.05; **P<0.01; ***P<0.001.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A3AR agonists as described herein increase the activity or expression of adenosine receptor 3 (A3AR). A3AR agonists may be small molecules that bind A3AR, or mRNA that increases A3AR expression. Examples of small molecule A3AR agonists include Piclidenoson and Namodenoson.

### Abbreviations

| | |
|---|---|
| A1AR | Adenosine A1 receptor |
| A3AR | Adenosine A3 receptor |
| AC | Anterior cingulate |
| ACAS | Asymmetrical carotid artery stenosis |
| AD | Alzheimer's disease |
| Ada | Adenosine deaminase |
| AP | Anterior-posterior |
| BAM | Border-associated macrophage |
| BCAO | Bilateral occlusion of the common carotid arteries |
| BCAS | Bilateral carotid artery stenosis |
| CC | Corpus callosum |
| DEG | Differentially expressed gene |
| DV | dorsoventral |
| EC | Endothelial cell |
| FACS | Fluorescence-activated cell sorting |
| FPKM | Fragments per kilobase million |
| FSC | Forward scattering |
| GFP | Green fluorescence protein |
| GPCR | G-protein coupled-receptor |
| GSEA | Gene set enrichment analysis |
| HPC | Hippocampus |
| L-R | Ligand-receptor |
| LRP1 | Lipoprotein receptor-related protein 1 |
| LTP | Long term potentiation |
| MBP | Myelin basic protein |
| ML | Medio-lateral |
| MRI | Magnetic resonance imaging |
| NOR | Novel Object Recognition |
| n.s. | Not significant |
| OPC | Oligodendrocyte progenitor cells |
| PBS | Phosphate-buffered saline |
| PFA | Paraformaldehyde |
| PFC | Prefrontal cortex |
| PFC-HPC | Prefrontal cortex-hippocampus |
| RRHO | Rank-rank hypergeometric overlap |
| SSC | Side scattering |
| TF | Transcriptional factor |
| TRAP | Translating ribosome affinity purification |
| VaD | Vascular dementia |
| WM | White matter |

### EXAMPLES

### Example 1: a Mouse Model that Recapitulates Multiple Features of Human (VaD)

Confluent infarcts were induced in the cerebral WM of C57 mouse brains by intracranial injections of the vasoconstrictor L-NIO (dihydrochloride). The resultant lesion was in several hundred micrometers along the medial-lateral and anterior-posterior axes in corpus callosum (CC) and cingulum. This lesion covered the most common sites of WM damage in human VaD. It produced significant axon and myelin loss, astrocyte and microglial activation, OPC proliferation, and pericyte responses.

To test the cognitive deficits in this model, a two-day Novel Object Recognition (NOR) test was employed. The memory impairment in VaD was significant, and the extent of deficit was inversely correlated to the WM lesion size. To explore memory deficits observed in VaD patients at early stages, the mice were tested in a contextual fear conditioning memory linking protocol, a hippocampal-dependent test that evaluates the linking or integration of context memories across a 5-hour interval. A significant impairment in this task in the VaD cohorts was observed.

VaD is fundamentally a disease of disconnection, damaging axonal tracts in the subcortical WM. In human VaD, periventricular WM hyperintensities may cause anatomic damage to the cingulum and CC, which connect the prefrontal cortex (PFC) to many brain regions including hippocampus (HPC) (a crucial region for learning and memory). To test this circuit in the mouse VaD model, its activation was measured and anatomical connectivity. Neuronal activation in PFC and HPC as measured by c-Fos (immediate early gene) levels quantified after memory retrieval (contextual fear conditioning), which indicated a significant neuronal activity decrease in AC in PFC, and in CA2/3 in HPC due to PFC-HPC circuit damage in VaD. Anatomical connectivity was labeled by injections of AAV1-CAG-Flex-EGFP and retroAAV-PKG-Cre into the dorsal-medial PFC and HPC respectively. CA1, CA2/3 and DG subregions in HPC were labeled with Cre recombinase. PFC neurons were double-labeled with Cre and GFP (green fluorescence protein) indicating a successful retrograde tracing. PFC axonal processes extended through subcortical WM and then projected toward the HPC. The ischemic lesion reduced the axon projection volume in WM and HPC, indicating the interruption of neural circuits that may subserve a memory function.

In multiple sclerosis, demyelinating lesions produce selective loss of neurons in overlying cortex expressing Cux2, a transcriptional factor (TF) and a cortical layer identity marker. Assessment of TFs and cortical layer markers in VaD revealed that Satb2 and Cux1, in lesion adjacent cortical layer VI, were significantly reduced, but not the layer VI marker Ctip2, the general neuronal marker NeuN, or the general cellular marker DAPI. In cortical regions that do not overlie the VaD lesion (layer II-V), no TFs were reduced, nor the number of neurons. These indicated that VaD lesions caused transcriptomic changes in the adjacent brain area without affecting the total number of neurons, which may serve as a marker of disease effect in connected neuronal circuits.

### Example 2: Cell-Type Specific RNA-Seq Reveals Activation of WM-Associated Aging Genes in VaD Mouse Model

To identify the transcriptome of the cells in the neurovascular nice, intersecting viral and mouse transgenic approaches were used in TRAP because it allows to study at great sequencing depth compared to scRNA or snRNAseq. Ribotag profiles the mRNA that is being transcribed, the “translatome”, which is more closely linked to the actual proteome in the cell than the total RNA sequencing of other approaches. To isolate mRNA from ECs, intersectional viral approach (PHP.eB-CAG-Flex-Rpl22-HA) and transgenic mouse strain (Tie2-Cre) were employed in conjunction with TRAP. Tbx18-CreER::Rpl22-HA mice were used to specifically isolate pericyte transcriptome using TRAP. Ng2-CreER::Rpl22-HA mice were used to specifically isolate OPCs. Lenti-GfaABC1D-Rpl22-HA was used to isolate astrocyte. Immunostaining with HA and cell type markers demonstrated specific labeling of EC (Glut1+), pericytes (Pdgfrβ+ or CD13+), OPCs (Olig2+Pdgfra+), astrocytes (GFAP+) respectively. Real-time PCR further confirmed the specific enrichment of each cell type. Co-staining of OPC (Pdgfrα+) and myelinated oligodendrocyte (Aspa+) showed that HA labeling was highly specific to OPCs in VaD, which may due to limited differentiation after injury. Although most canonical cell type specific markers reported in cortex were enriched after TRAP, many of them were diminished in WM cells, e.g. OPC genes Chga, Nptx2, Olfm1, Grik3, and Garba3, etc., were reduced after TRAP in Ng2-CreER::Rpl22-HA mice, suggesting a distinct transcriptomic profile of WM cells. PCA analysis distinguishes the input and pulldown fractions from the control and VaD samples into different clusters, establishing the validity of the pulldown approach. Differentially expressed genes (DEGs) were identified with an FDR<0.1.

To isolate microglia, a fluorescence-activated cell sorting (FACS) protocol for small WM regions using CX3CR1, CD11b, and CD45 antibodies was applied. Based on cell scatter size, microglial obtained from the control brain was designated as P2, while the same fraction and an additional fraction from VaD brain are named P2 and P4 respectively. RNA was extracted and sequenced, then clustered in PCA analysis. Border-associated macrophage (BAM) genes were trace or undetectable indicating that the all FACS sorted cells were pure microglia. Thousands of microglial DEGs were identified (VaD.P4 vs. Con.P2, and VaD.P2 vs. Con.P2). Taken together, most of the VaD related DEGs were cell type specific, and half of which are dysregulated in only one cell type in both mouse and human.

Glial aging is particularly accelerated in WM compared with cortical regions, while corpus callosum shows the most profound and earliest shifts towards aging. Substantial numbers of WM-associated aging genes were changed in 5 types of glial cells, mostly up-regulated, indicating that the VaD model in the young adult mouse caused a molecular expression profile that is seen in aging. Microglia exhibited the most unique shift of WM-associated aging genes, and most substantial fold change in VaD. Notably, the cluster of P4 microglia, exhibited a bigger number and greater change of WM-associated aging genes compared to P2-cluster microglia. A key WM-associated aging gene, C4b, which is a complement component and major schizophrenia risk factor, is specifically up-regulated in OPC and microglia in VaD. Function analysis of KEGG pathways showed that microglial transcriptomic changes in the VaD model were associated with dysregulation of major molecular pathways, including those involved in Alzheimer's disease. GO enrichment analysis also indicated that microglia in VaD model were involved in dysregulated molecular functional pathways including ATP binding.

Many cell types have specific transcriptomes in distinct brain regions. A set of cell-type and WM-specific TFs/marker genes were identified. Foxc1, Sox4, Atoh8, and Zfp871 are examples of distinct WM cell-type specific TFs compared to cortex and whole brain. TFs are crucial for regulating gene expression. These findings suggest a distinct transcriptional control profile of WM cells against other brain regions. Cell-type marker genes specific for WM were also identified. Tmem212, Slc1a1, Thbs4, and Nav3 are highly expressed in WM EC, OPC, astrocyte, and microglia respectively but not in cortical datasets. Tmem212 is related to cerebral small vessel disease and associated with enlarged periventricular space in magnetic resonance imaging (MRI) in aged populations. VaD significantly increased Tmem212 expression in mouse [FPKM, 61.3±10.4 (control) vs 118.5±8.3 (VaD)]. Rank-rank hypergeometric overlap (RRHO) analyses comparing WM cell type transcriptomes with cortical and whole brain show lower similarity of transcriptional profiles in EC and microglia specifically. Furthermore, gene set enrichment analysis (GSEA) showed distinctive gene markers and functional hallmarks of WM EC and microglia compared with the same cell types in cortex. These results highlight the importance of assembling a WM-specific cell transcriptome as a critical first step in identifying disease-associated genes in VaD, as well as other WM diseases.

### Example 3: Constructing a Map of the Intercellular Interactome in VaD

To determine the cell-cell signaling systems within the WM neurovascular niche, the VaD cell-type specific transcriptomes were analyzed for L-R systems that are conjointly and differentially regulated. First, a L-R database was assembled by merging three major L-R libraries covering 4053 human and 2032 mouse L-R pairs/complexes, which is currently the largest of its kind. Subsequently, the number of ligand and receptor genes related to mouse VaD were identified in each cell type. For example, from the microglia RNA-Seq data in mouse VaD, 118 ligands were identified including 84 from human L-R pool and additional 34 from mouse L-R pool. Human VaD related L-R genes were also identified from our published snRNA-Seq dataset.

To determine the intercellular interactome during VaD recovery, cells were linked together via L-R interactions, which can be visually presented by Circos plots. In mouse VaD, the top microglial ligands (LogFC>2 or <−1.5) identified interfacing the human L-R pool, were connected towards corresponding receptors in other cell types, where SPP1 was the most increased ligand, and FARP2 was the most decreased ligand. In human VaD, the microglial ligands were identified interfacing the human L-R pool and connected towards corresponding receptors in other cell types.

### Example 4: Categorization of L-R Genes as Potential Candidates for VaD Treatment

In order to screen potential targets for VaD treatments, a series of molecular identity and significance criteria was used to categorize the DEGs in ascending order of priority for study. First, the candidate must be a ligand (Tier 5). Second, the ligand candidates from the mouse VaD data set had to also be significantly regulated in human VaD (Tier 4). Next, ligand candidates comparably changed in only one or two cell types were prioritized (Tier 3). Then, those with significant change in corresponding receptors were identified (Tier 2). Finally, the candidates with a reported function related to neurological activity were classified (Tier 1). In this selection scheme, Tier 1 L-R genes are the most attractive candidates under the scope of an intercellular interactome for VaD study.

To classify the L-R candidates in terms of functions, ECM and G-protein coupled-receptor (GPCR) were identified as two major groups of candidates. Microglia and pericyte are more involved in the regulation of ECM components, including the Tier 1 candidates: ECM regulating gene Entpd1 (CD39) and Serpine2 (PN-1). Microglia also exhibited higher dysregulation of GPCRs, including the Tier 1 receptor: Adora3 (A3AR).

### Example 5: Serpine2-Lrp1 Intercellular Pathway Inhibits OPC Maturation in VaD Tissue Repair

Serpine2 was significantly increased in pericytes and microglia in mouse VaD and astrocytes in human VaD. Serpine2-encoded protease nexin-1 (PN-1) plays a role in multiple sclerosis and Alzheimer's disease. It binds to low density lipoprotein receptor-related protein 1 (Lrp1) to mediate functions in cancer and vascular biology. Lrp1 expression in OPC is higher than in myelinated oligodendrocytes. Its expression in OPC was further validated using immunostaining. RNA-Seq showed a significant increase of Lrp1 expression in VaD in OPC. Therefore, the Serpine2-Lrp1 axis was overall up-regulated and may play a role in the differentiation of OPCs into myelinating oligodendrocytes which is required for VaD tissue repair. To investigate the function of Serpine2 on OPC differentiation, remyelination was evaluated in Serpine2 KO mice during VaD recovery. Homozygous knockouts were excluded, considering their significant epileptic activity and impairment of memory associated-long term potentiation (LTP). Immunostaining of myelin basic protein (MBP) showed that the reduction of myelin volume in VaD was ameliorated by reduced Serpine2 expression in heterozygous siblings. Quantification of myelinating oligodendrocytes (Aspa+Olig2+EdU+) differentiated from newly formed OPCs (Olig2+EdU+) in the infarct core indicated that the differentiation of newborn OPC after VaD was increased by Serpine2 deficiency. Strikingly, the NOR task showed that reduced Serpine2 expression rescued the memory deficit in VaD. To summarize, Serpine2 plays a key role in the WM damage that underlies VaD, and its loss in this model promotes progenitor responses toward myelination and restores memory function.

### Example 6: CD39-A3AR Intercellular Pathway is a Potential Target for Tissue and Behavior Repair in VaD

CD39 (Entpd1) is an ectonucleotidase that plays a significant role in regulating the balance of extracellular ATP. It has potential as a cancer immunotherapeutic target. Blockade of CD39 may prevent cancer cell adhesion to the ECM. Cell type-specific RNA-Seq shows that CD39, and its receptor A3AR (Adenosine A3 receptor, gene name Adora3) were specifically expressed in microglia, and significantly reduced in VaD. CD39 plays a role in the negative feedback control of neuronal activity by microglia through A1AR. However, A1AR is not significantly changed in VaD. Extracellular signaling in CD39-A3AR occurs as ATP is converted by CD39 to ADP and AMP; AMP is converted by CD73 (Nt5e) to adenosine; adenosine activates A3AR and is degraded by Ada. RNA-Seq data indicated low expression of Nt5e (FPKM≤3), and no significant change in Nt5e and Ada. These data position extracellular ATP and its regulation by CD39 in VaD.

The cellular localization and protein levels in the CD39/A3AR system were determined in mouse and human, and as a function of age and VaD. Co-staining of CD39 with cell specific markers show that CD39 was specifically expressed in microglia and EC. Aging alone (3-mo to 30-mo in the mouse) did not lead to reduction of CD39 expression, while aging and VaD together significantly reduced CD39 expression in EC and microglia. Human VaD snRNA-Seq showed that microglial and endothelial CD39 expression in the WM lesion core and adjacent area was significantly reduced. Immunohistochemistry further validated a significant reduction (50.6%) of CD39 expression in VaD brains. In contrast, the immunoreactivity of Glut1 (EC marker) was reduced only by 20.3%, which may be due to ischemic injury in vessels, while Iba1 (microglia marker) was not significantly changed. Therefore, reduced CD39 immunoreactivity in human VaD is caused by its impaired expression in EC and microglia, including some decreased volume of blood vessels. Notably, the reduction of CD39 expression was inversely related to age in human VaD, which indicates that aging and VaD synergistically reduce CD39 expression.

These data suggest that intercellular CD39-A3AR signaling is an endogenous mechanism associated with VaD: Endothelial and microglial CD39 enhance the conversion of ATP into adenosine, which modulates microglia through A3AR; Infarct and aging together reduce CD39 expression in EC and microglia during VaD, which lead to impaired signaling to microglia in the infarct core and adjacent area, and thereafter retarded tissue and behavioral repair. In the studies of ischemic stroke, A3AR plays a neuroprotective role in the central nervous system. Chronic administration of an A3AR agonist before global brain ischemia improved post-ischemic cerebral blood circulation, survival, and neuronal preservation. However, the possibility of a delayed action of this signaling axis during the progression of WM ischemia/VaD has not been determined.

Immunostaining validated the expression of A3AR in WM microglia and the reduction of Adora3 in microglia was validated by RNAScope. To investigate whether increasing an affected CD39-A3AR signaling is beneficial to VaD recovery, CF101, an A3AR-specific agonist, was delivered either acutely or in a delayed manner after VaD induction. CF101 is 50-fold more potent in A3AR action than with A1AR and A2bAR and has been safely used in humans in recent phase III clinical trials for psoriasis. Acute administration of CF101 from 30-min post stroke, for 1-2 weeks, by direct infusion into the lateral ventricle, reduced blood brain barrier (BBB) leakage. However, unlike large artery stroke, periventricular WM microinfarcts in the human are often asymptomatic and progresses for years before development of VaD symptoms, so that any medication in VaD is likely to be a delayed treatment. Delayed CF101 delivery produced a significant reduction of lesion size. Although measures of axonal projections were not changed, markers of myelination were significantly increased in affected WM by delayed CF101 treatment. To the best of our knowledge, this is the first finding of a drug that reduces lesion progression or enhances tissue repair in such a delayed treatment. Importantly, the NOR memory test showed that CF101 significantly reduced memory deficits in VaD animals, which also included restored c-Fos expression in PFC (AC) and HPC (CA2/3).

Human VaD is associated with gait and motor abnormalities that predict dementia status and are associated with morbidity. Using grid-walking, which tests limb motor control, delayed CF101 treatment successfully rescued the motor deficit in VaD. The reduced expression of neuronal transcription factors, Satb2 and Cux1, in the deep layers of motor somatosensory cortex, adjacent to the WM VaD lesion is a marker of secondary injury effect in this model. Immunostaining using the animal cohorts after grid-walking tests showed that the reduced Satb2 and Cux1 expression, were significantly ameliorated by CF101 treatment.

### Example 7: a Mouse Model that Recapitulates Multiple Features of Human Vascular Dementia (VaD)

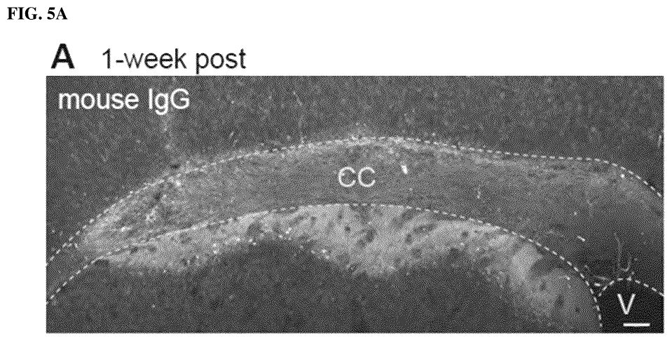

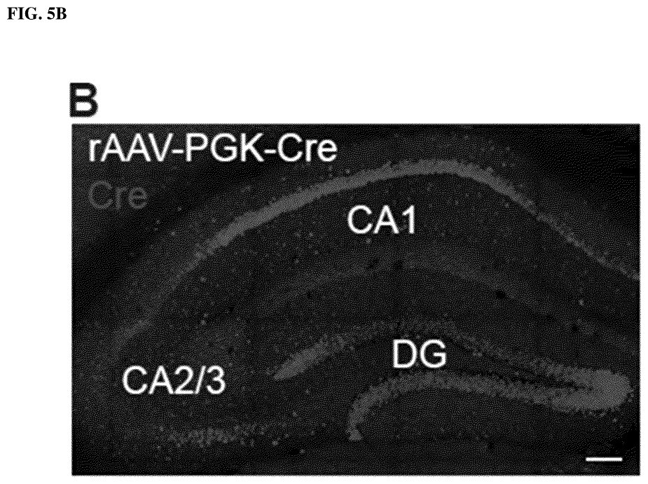

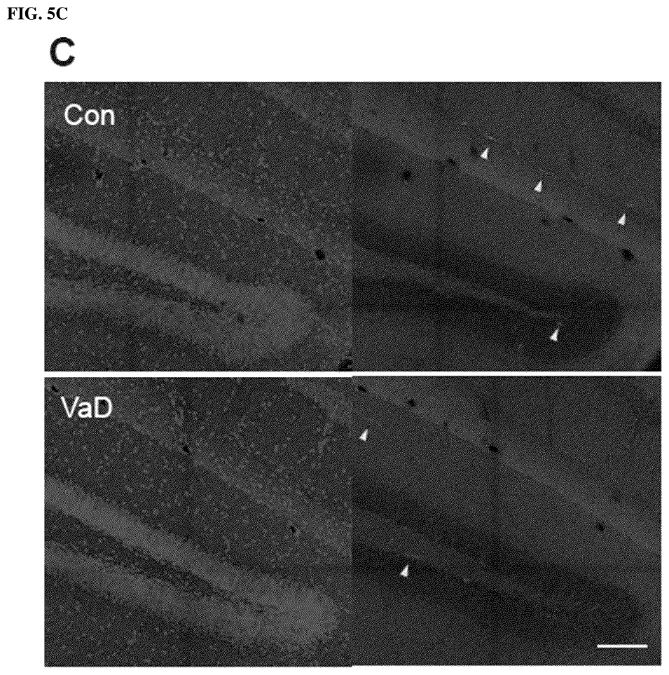

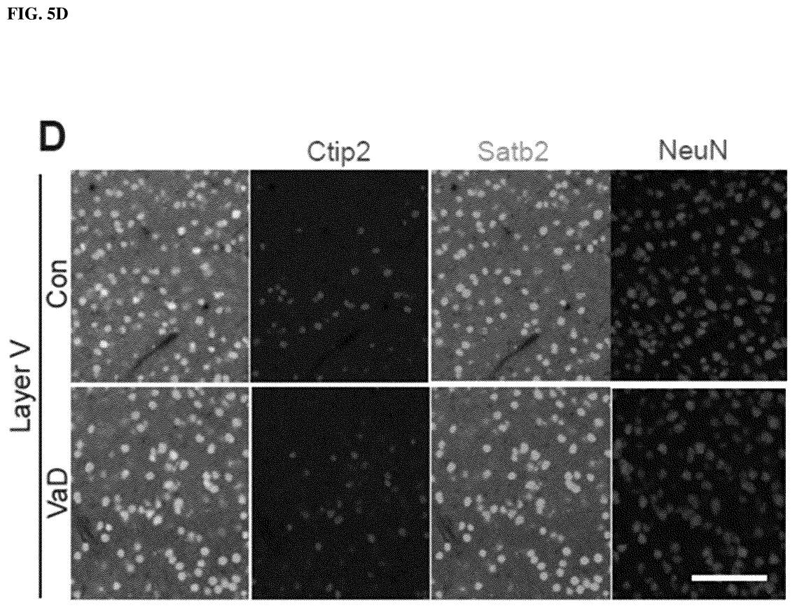

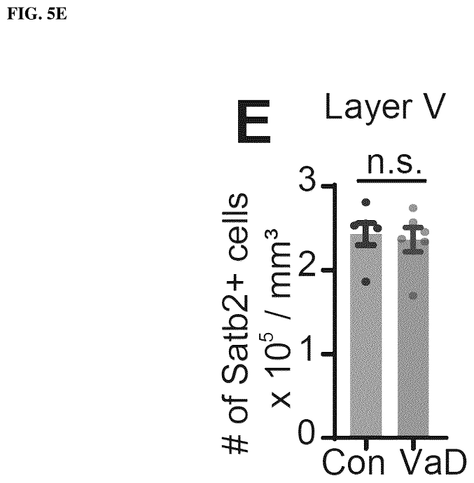

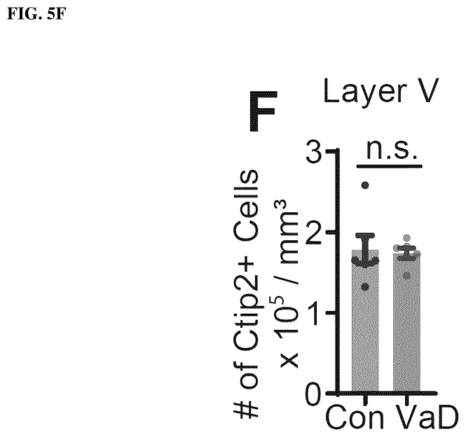

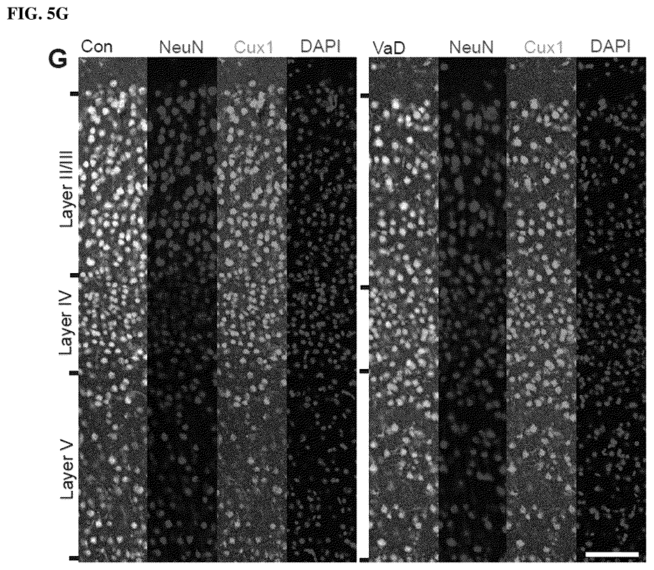

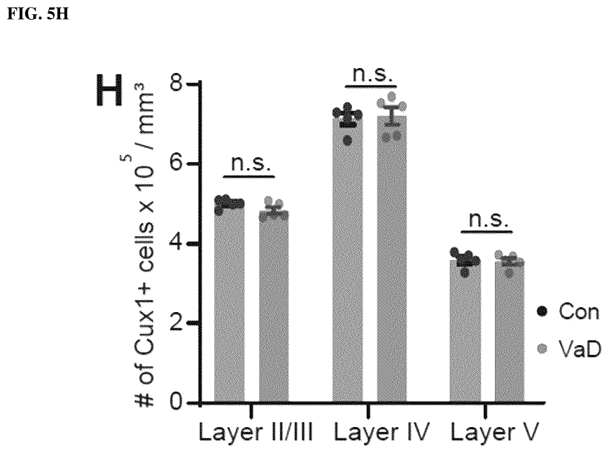

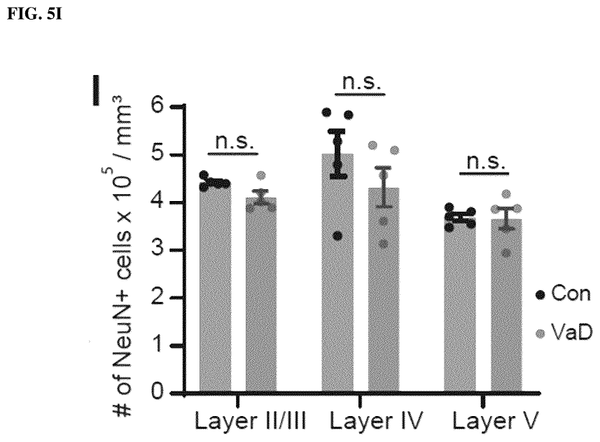

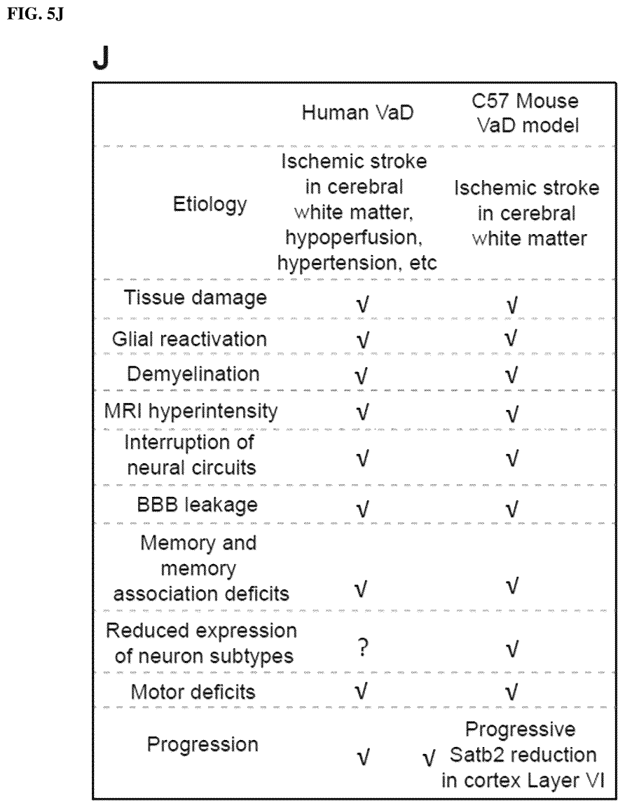

Similar to immunocompromised mice, multiple infarcts were induced in the cerebral white matter (WM) of C57 mouse brains by intracranial injections of the vasoconstrictor L-NIO (dihydrochloride) (FIG. 3A). A unilateral lesion was used to investigate the axon/myelin damage by comparison to the unaffected contralateral hemisphere, and to observe motor function impairment in the affected forelimb. This lesion led to WM hyperintensity in MRI (FIG. 3A, right panel), which correlated in location to the most common sites of WM damage in human VaD. This ischemic lesion damaged the blood-brain barrier (BBB) (FIG. 5A) and produced significant microglial activation (FIGS. 3B and 3C), dilation of capillary blood vessel (FIGS. 3B and 3D), pericyte/fibroblast responses (FIGS. 3B and 3E), oligodendrocyte progenitor cell (OPC) proliferation (FIGS. 3B and 3F), and changes in astrocyte (FIGS. 3B and 3G). The VaD lesion was distinguished by axon damage (NF160), myelin loss (myelin basic protein/MBP), and astrocyte activation (GFAP) (FIG. 3H). The lesion size at 1-month post ischemia is ˜0.09 square millimeter (FIG. 3I).

VaD patients show significant impairments in cognitive functions. To test cognitive deficits in this model, a two-day Novel Object Recognition (NOR) test (FIG. 3J) was employed. The memory impairment was significant at 1-month post VaD induction (FIG. 3K), and the cognitive function was inversely correlated to the WM lesion size (FIG. 3L). To explore whether this VaD model shows associative memory (i.e. memory linking) deficits observed in the aging population, the mice were tested in a contextual fear conditioning memory linking protocol (FIG. 3M), a hippocampal-dependent test that evaluates the linking or integration of context memories across a 5-hour interval. A significant impairment in this task in the VaD cohorts was observed (FIG. 3N).

VaD is fundamentally a disease of disconnection, damaging axonal tracts in the subcortical WM. In human VaD, periventricular WM hyperintensities reflect anatomic damage to the cingulum and corpus callosum, which connect the prefrontal cortex (PFC) to many brain regions including hippocampus (HPC) (a crucial region for learning and memory). To test the PFC-HPC circuit in the mouse VaD model, its anatomical connectivity was measured and activation. To validate the damage of PFC-HPC circuit in VaD, anatomical connectivity was labeled by injections of AAV1-CAG-Flex-EGFP and retroAAV-PKG-Cre into the dorsal-medial PFC and HPC respectively (FIG. 3O). CA1, CA2/3 and DG subregions in HPC were labeled with Cre recombinase (FIG. 5B). PFC neurons were double-labeled with Cre and GFP (green fluorescence protein) (FIGS. 3P and 3Q) indicating a successful retrograde tracing. PFC axonal processes extended through subcortical WM (FIG. 3R) and then projected toward the HPC (FIGS. 3T and 5C). The ischemic lesion reduced the axon projection volume in WM (FIG. 3S) and HPC (FIG. 3U), indicating the interruption of neural circuits that may subserve a memory function. Neuronal activation in PFC and HPC was further measured by c-Fos (immediate early gene) expression quantified after memory retrieval (contextual fear conditioning) (FIGS. 3V-3Y), which showed a significant neuronal activity decrease in anterior cingulate (AC) in PFC (FIGS. 3W and 3X), and in CA2/3 in HPC (FIGS. 3W and 3Y) due to PFC-HPC circuit damage in VaD.

### Example 8: VaD Mouse Model Show Sustained Behavior Deficits and the Nature of Progression

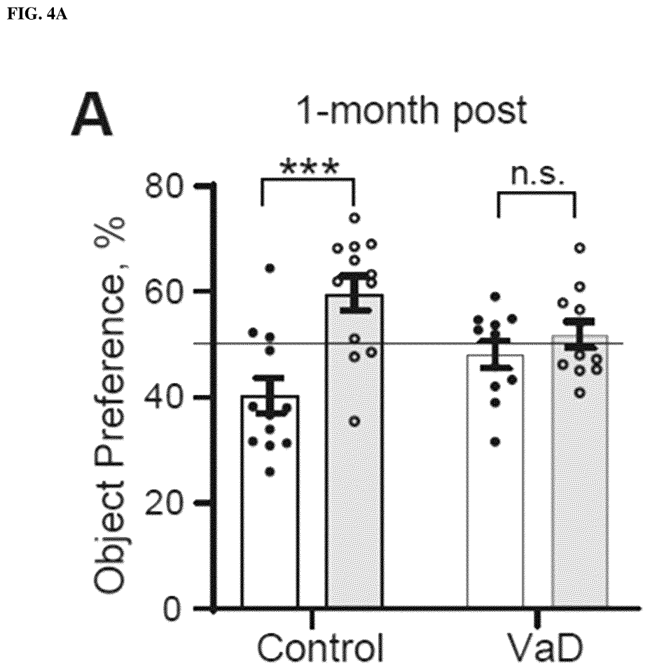

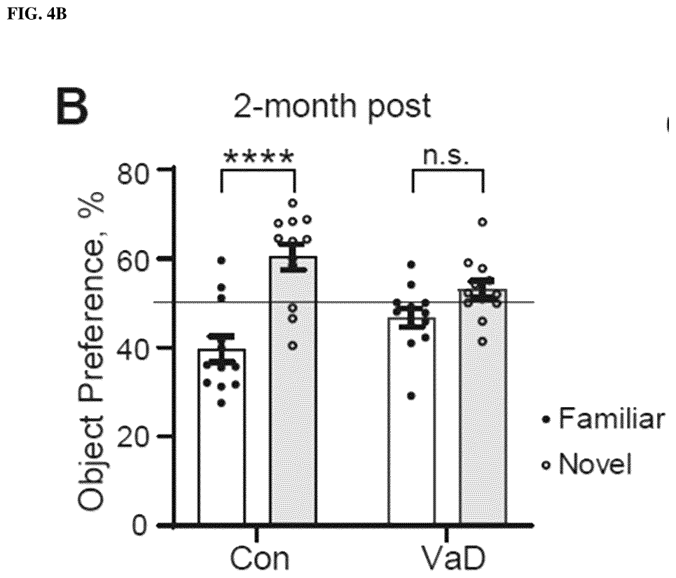

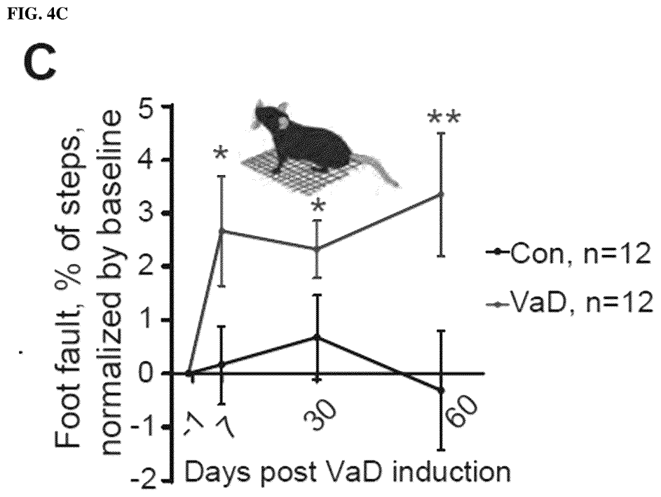

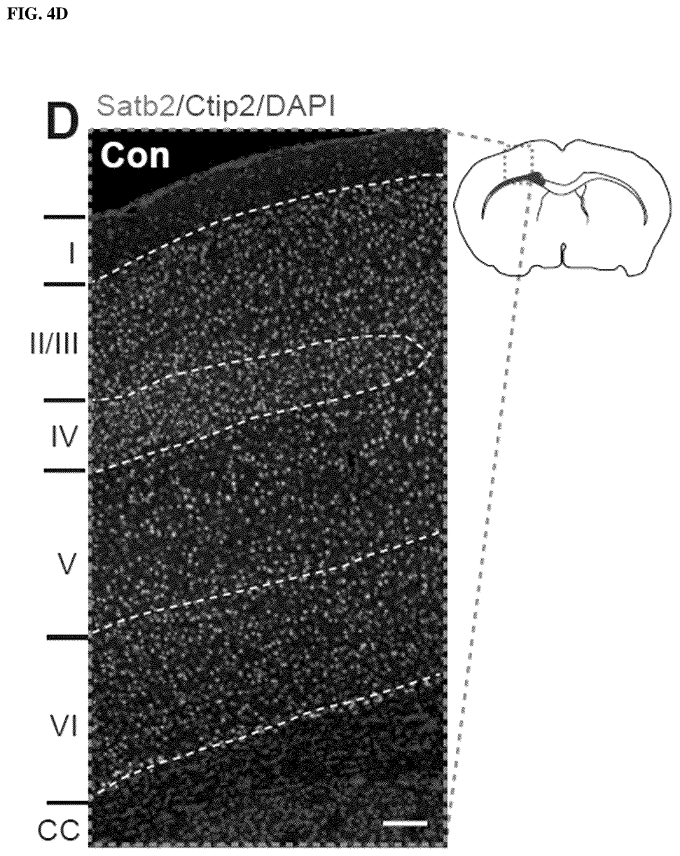

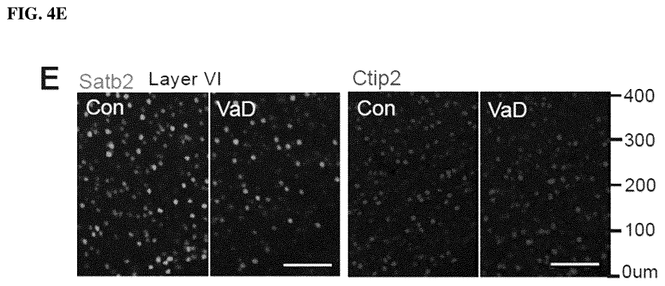

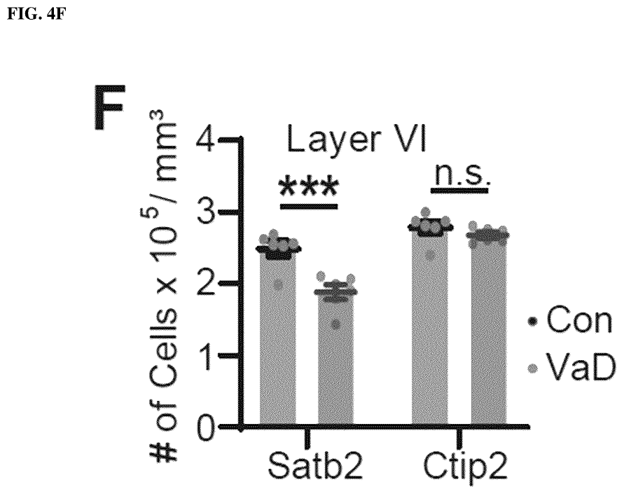

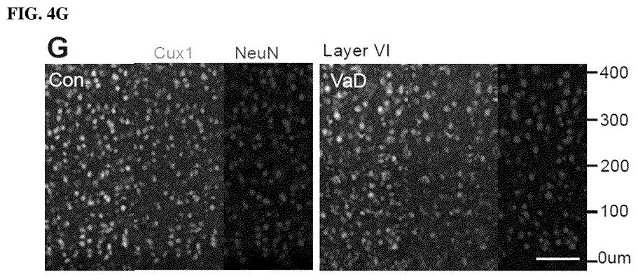

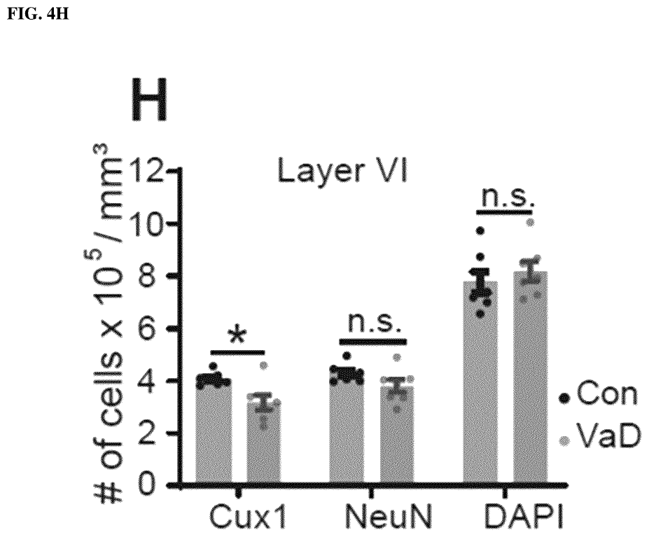

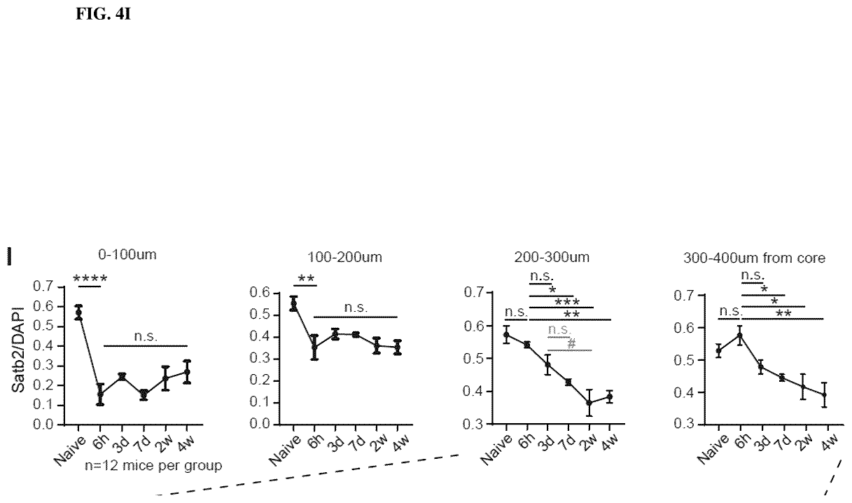

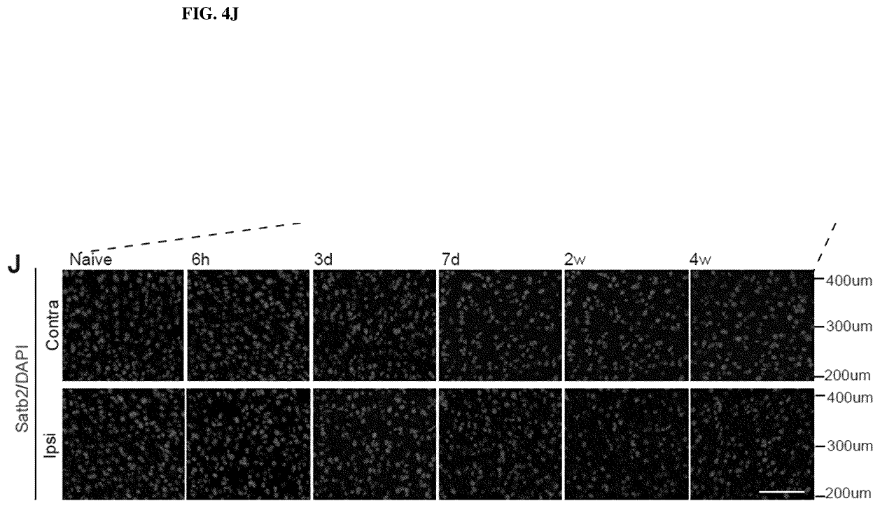

Consistent with previous findings in immunocompromised mice, the VaD model in C57 mice exhibited impaired memory in the NOR test for at least two months (FIGS. 4A and 4B). Motor abnormalities have also been observed in VaD patients. Similarly, motor deficits previously reported in the grid walking test using immunocompromised mice were replicated in C57 mice on days 7, 30, and 60 following VaD induction, with a progressive worsening trend (FIG. 4C).

Because WM demyelination is largely observed in VaD, and demyelinating WM lesions in multiple sclerosis cause selective loss of neurons in overlying cortex expressing Cux2, a transcription factor (TF) and a cortical layer identity marker, the expression of TFs and cortical layer markers in VaD were further assessed. Remarkably, TF Satb2, but not deeper layer marker Ctip2 was significantly reduced in lesion-adjacent cortical Layer VI (FIGS. 4D-4F). Similarly, TF Cux1, but not the general neuronal marker NeuN or the general nucleus dye DAPI, was significantly reduced in Layer VI in VaD (FIGS. 4G and 4H). In cortical Layer V which does not overlie the VaD lesion, neither Ctip2 nor Satb2 expression was affected (FIGS. 5D-5F). Cux1 and NeuN were also not affected in Layer II-V (FIGS. 5G-51). These data indicated that VaD lesions caused TF changes in the adjacent brain area without affecting the total number of neurons, which may serve as a marker of disease effect in connected neuronal circuits.

Remarkably, the reduction of Satb2 in distal Layer VI exhibited a progressive, time- and spatially dependent pattern (FIGS. 4I and 4J). In the area proximal (0-100 and 100-200 μm) to the VaD lesion, the expression of Satb2 decreased as early as 6-hour post-ischemia and did not recover for at least 4 weeks (FIG. 4I). In an area less proximal (200-300 and 300-400 μm) to the VaD lesion, the Satb2 expression did not decrease at 3-day post-ischemia, but was significantly reduced on day 7, and progressively worsened to 2- and 4-weeks after the lesion was induced (FIGS. 4I and 4J). As this cortical region covers motor and somatosensory areas, the reduced Satb2 expression may be related to impaired motor functions. Accordingly, motor deficits were not observed in grid walking test on the 4th day post-VaD induction (FIG. 4C), while significant higher percentage of foot faults were shown on day-7, day-30, and day-60 in a likely worsening trend (FIG. 4C), similar to previous findings in the VaD model using immunocompromised mice. These data indicate that the lesion progresses in this model, with greater reductions in neuronal TF expression over time. All the characteristics of the mouse VaD model compared with human VaD are listed in FIG. 5J and Table 1.

TABLE 1
Summary of of the VaD mouse model with human
VaD pathological and clinical characteristics

| | | C57 Mouse |
|---|---|---|
| | Human VaD | VaD model |
| Etiology | Ischemic stroke | Ischemic stroke |
| | in cerebral | in cerebral |
| | white matter, | white matter |
| | hypoperfusion, | |
| | hypertension, etc | |
| Tissue damage | √ | √ |
| Glial reactivation | √ | √ |
| Demyelination | √ | √ |
| MRI hyperintensity | √ | √ |
| Interruption of | √ | √ |
| neural circu | | |
| BBB leakage | √ | √ |
| Memory and | √ | √ |
| memory | | |
| association deficits | | |
| Reduced expression | ? | √ |
| of neuron subtypes | | |
| Motor deficits | √ | √ |
| Progression | √ | √ Progressive Satb2 |
| | | reduction in Cortex Layer VI |

### Example 9: Cell-Type Specific RNA-Seq Reveals VaD Related Gene Changes and Unique WM Transcriptome

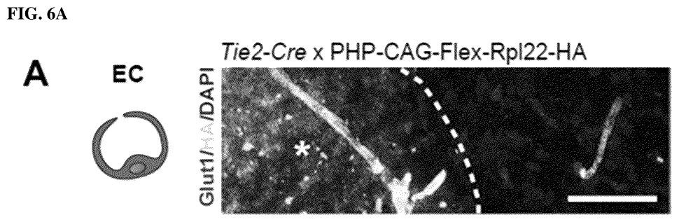

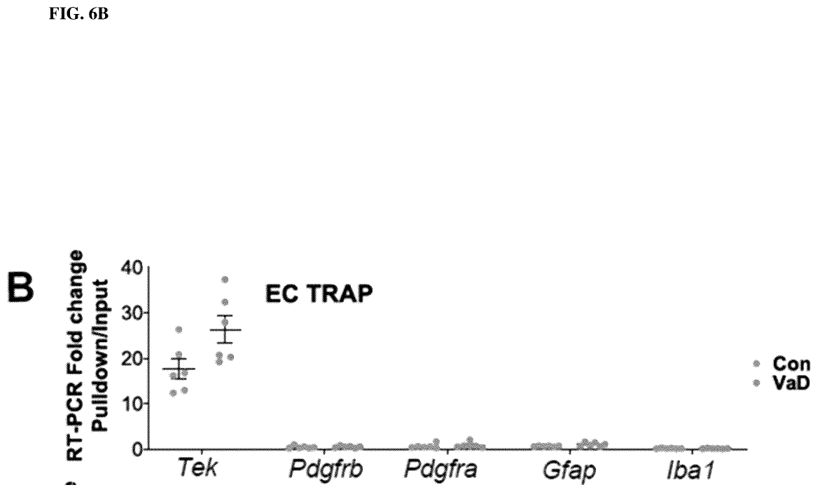

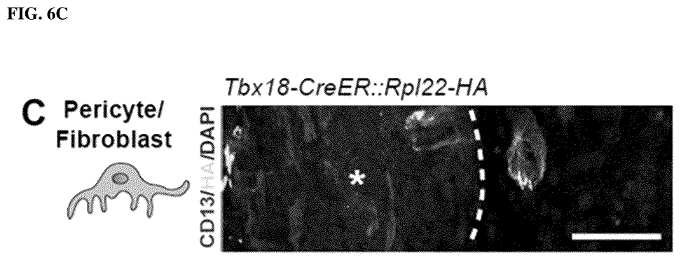

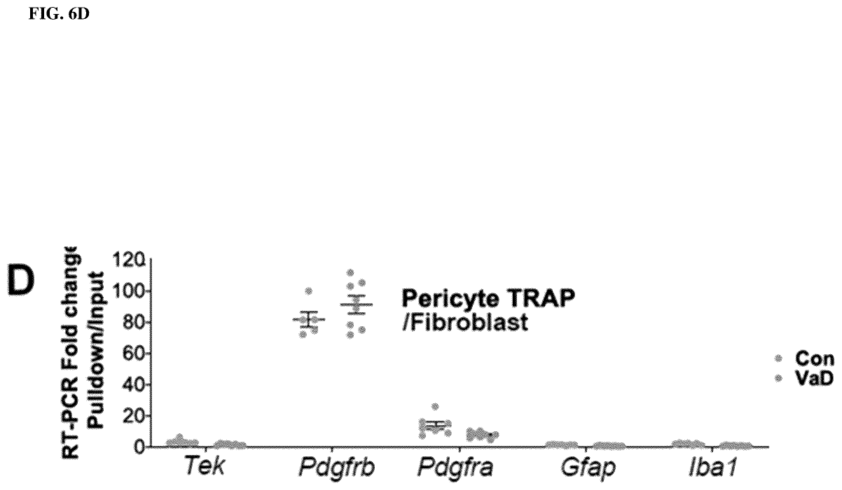

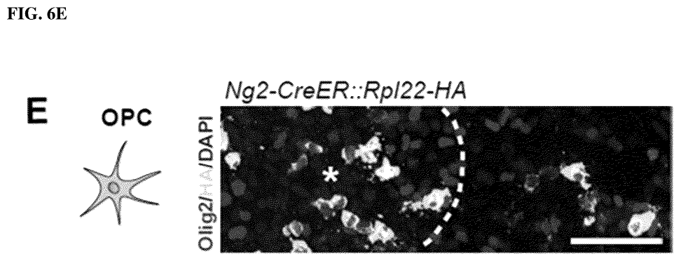

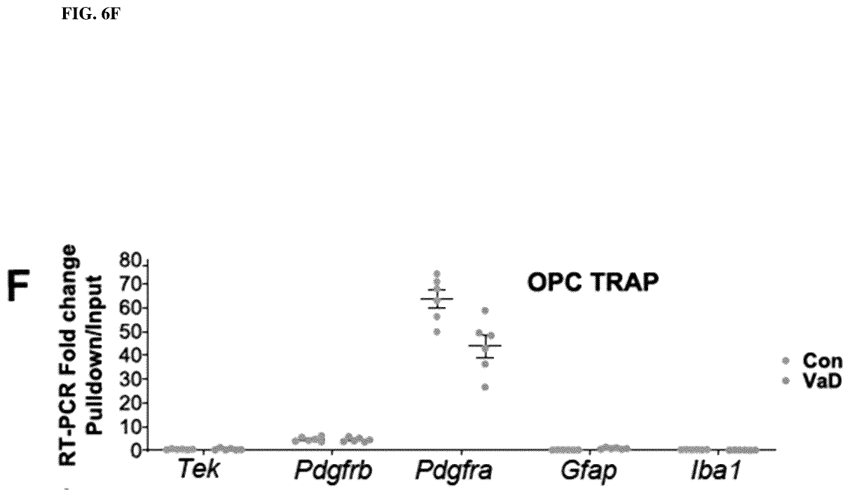

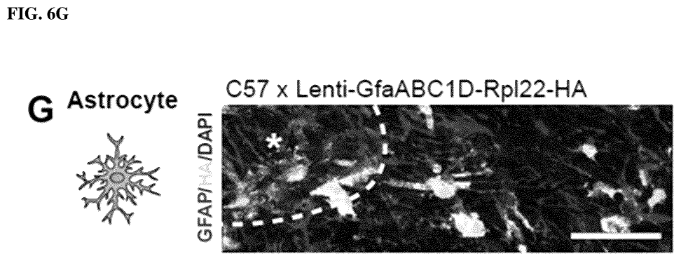

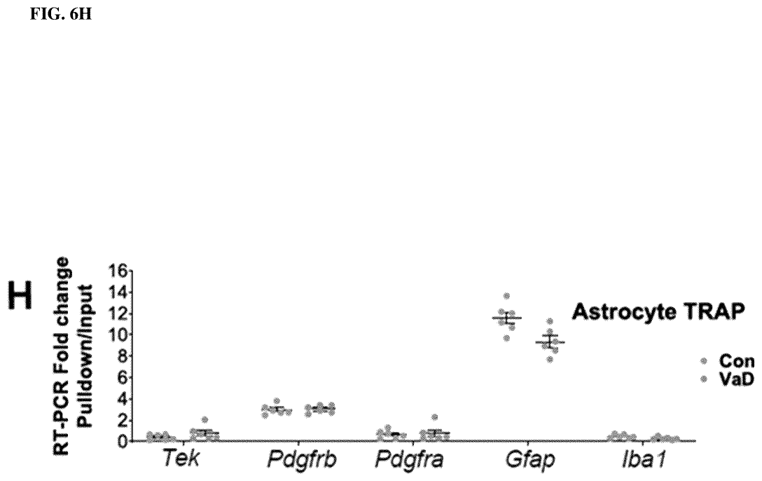

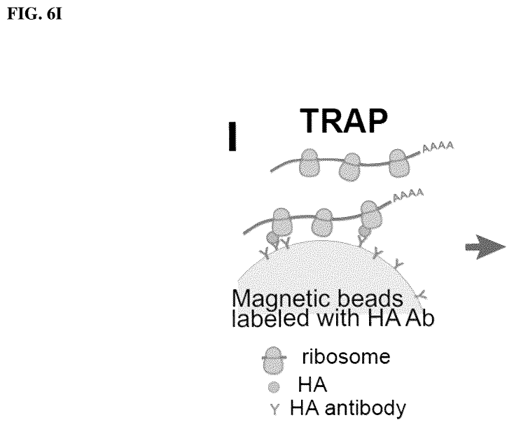

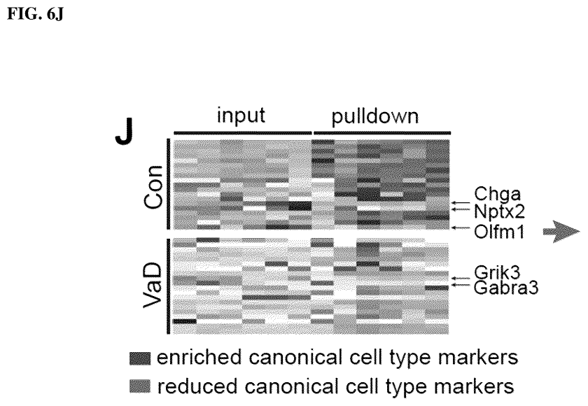

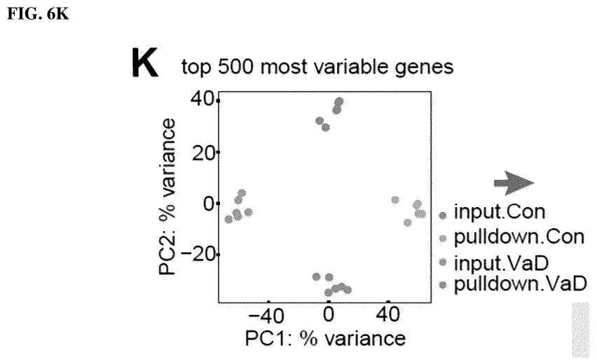

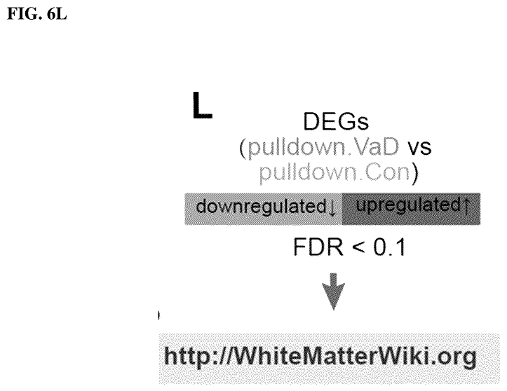

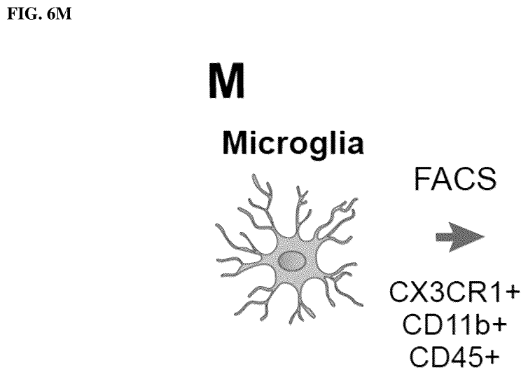

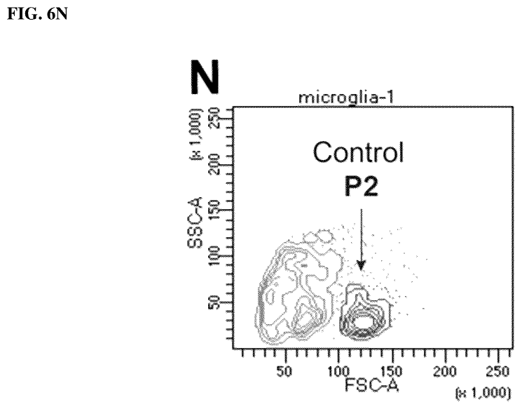

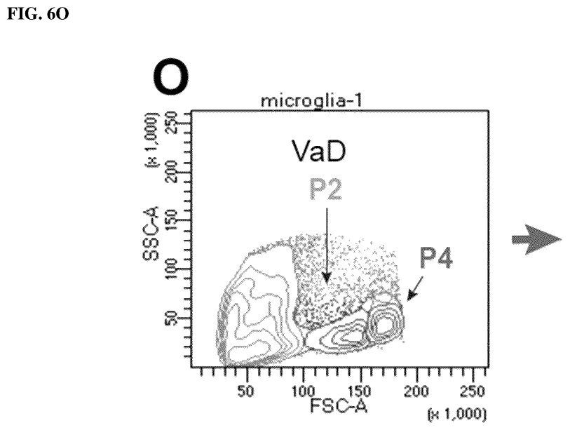

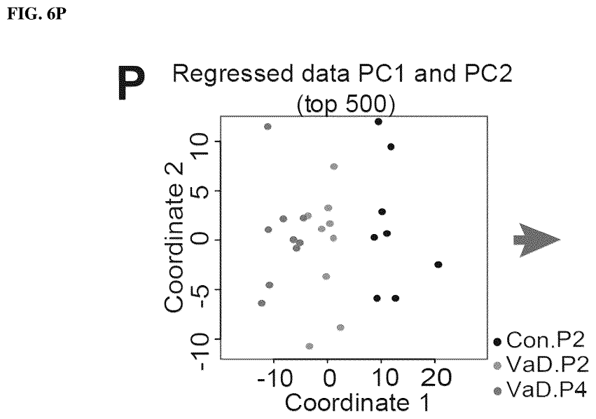

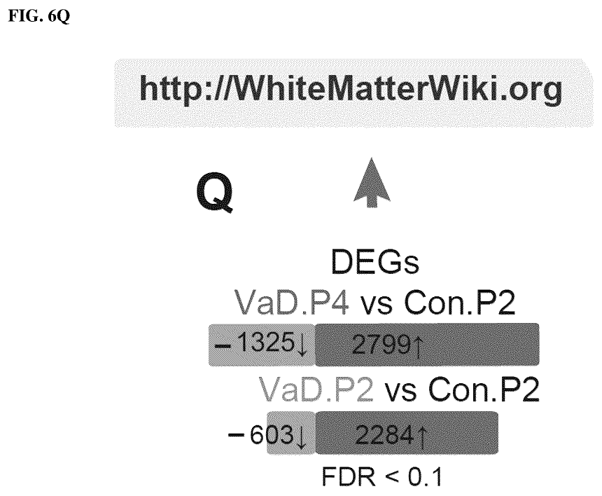

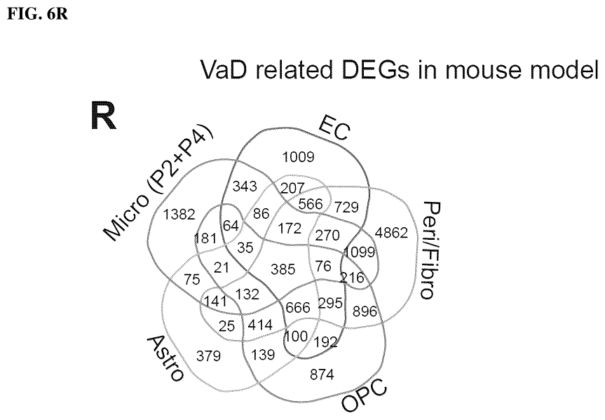

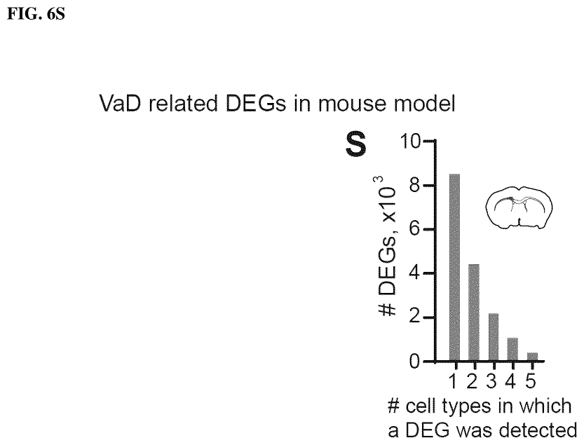

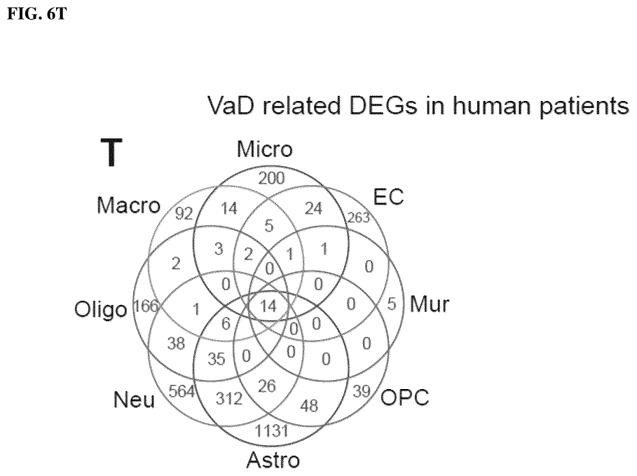

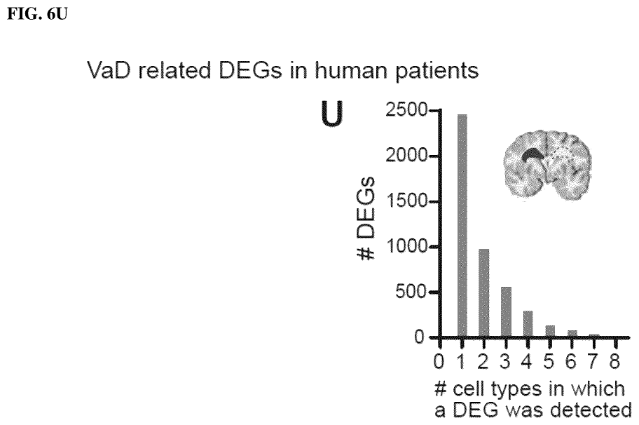

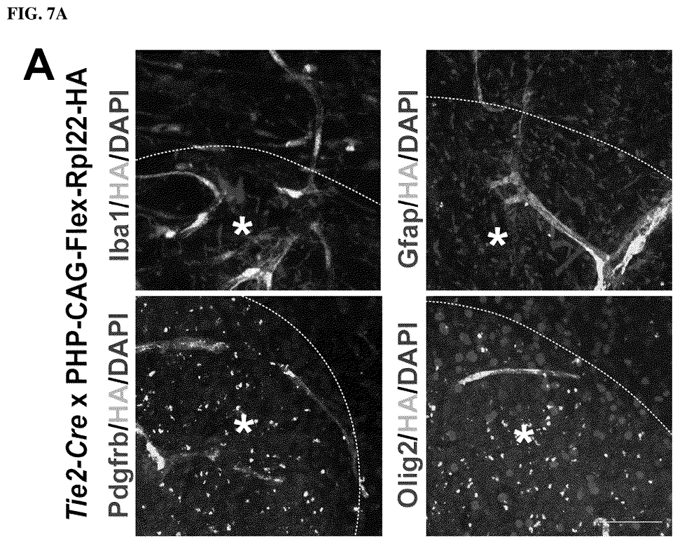

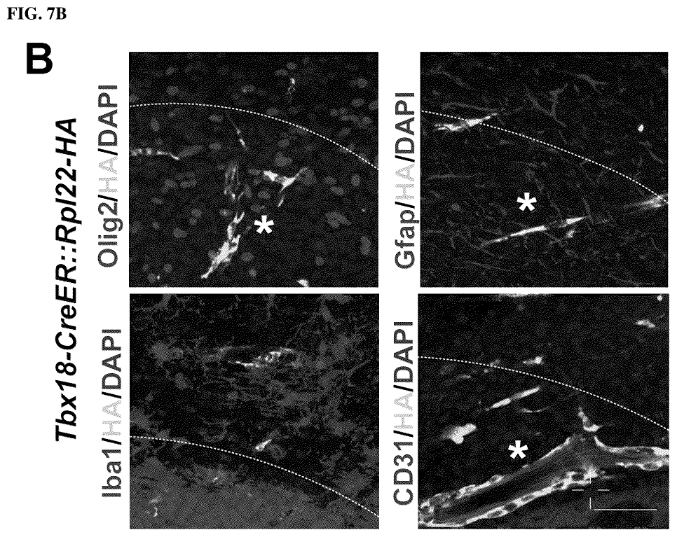

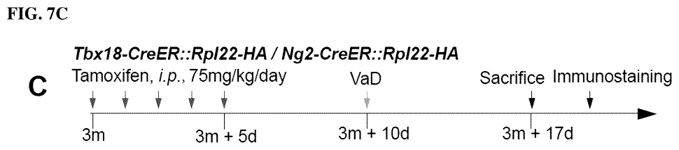

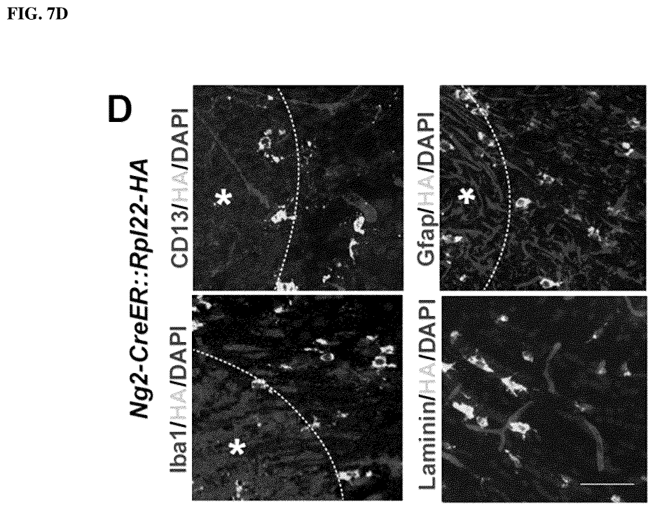

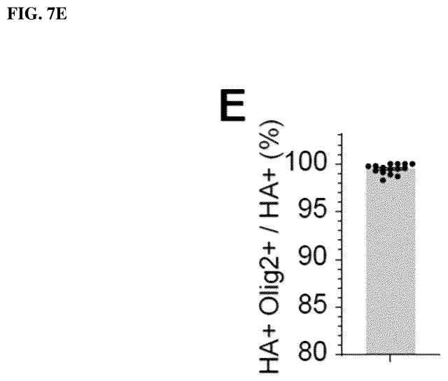

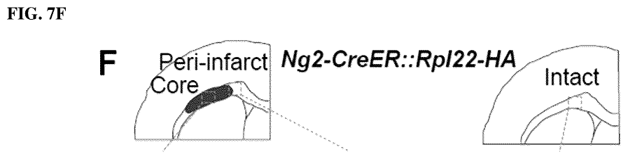

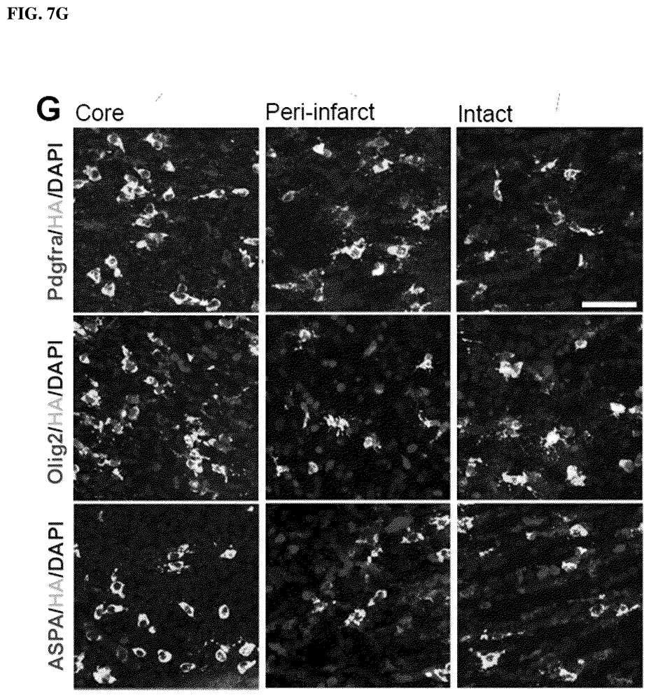

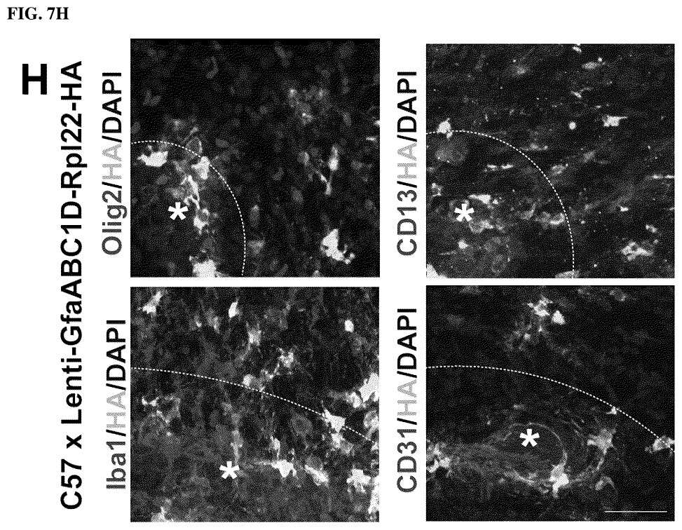

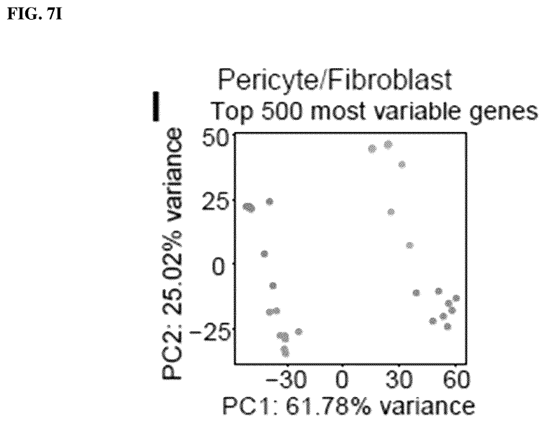

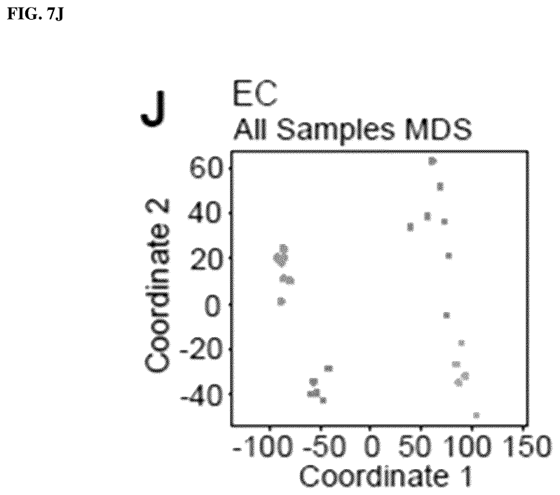

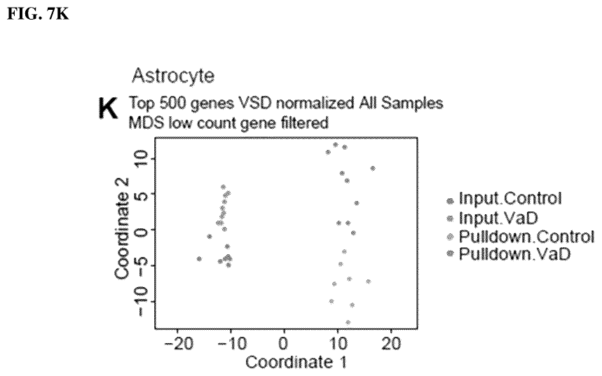

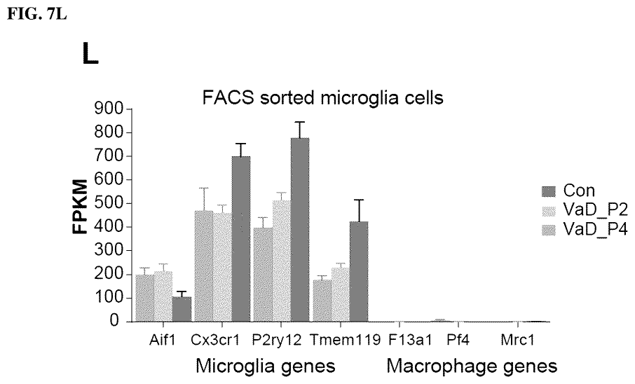

To identify the transcriptome of the cells in the neurovascular niche, intersecting viral and mouse transgenic approaches were used to apply translating ribosome affinity purification (TRAP), as it allows interrogation at a greater sequencing depth compared to scRNA or snRNAseq, especially for small and lipid tissue like WM in mouse brain. Ribotag profiles the mRNA that is being translated, the “translatome”, which is more closely linked to the actual proteome in the cell than the total RNA sequencing of other approaches. To isolate mRNA from endothelial cells (ECs), intersectional viral approach (PHP.eB-CAG-Flex-Rpl22-HA) and transgenic mouse strain (Tie2-Cre) were employed in conjunction with TRAP (FIGS. 6A, 6B, and 6I). Tbx18-CreER::Rpl22-HA mice were used to specifically isolate pericytes/fibroblasts transcriptome (FIGS. 6C, 6D, and 7C). Ng2-CreER::Rpl22-HA mice were used to specifically isolate OPCs (FIGS. 19E, 19F, 20C). Lenti-GfaABC1D-Rpl22-HA was used to target astrocytes (FIGS. 6G and 6H). Immunostaining of HA and cell type markers demonstrated specific labeling of EC (Glut1+), pericytes/fibroblasts (CD13+), OPCs and the derived oligodendrocyte lineage (Olig2+), as well as astrocytes (GFAP+) (FIGS. 6A, 6C, 6E, 6G, 7A, 7B, 7D, 7E, 7G, and 7H). Real-time PCR further confirmed the specific enrichment of each cell type (FIGS. 6B, 6D, 6F, and 6H). Co-staining of OPC (Pdgfra+) and myelinated oligodendrocyte (Aspa+) showed that HA labeling was highly specific to OPCs in VaD, which may due to limited differentiation after injury (FIGS. 7F and 7G). Although most canonical cell type specific markers reported in cortex were enriched after TRAP, many of them were diminished in WM cells. For example, OPC genes Chga, Nptx2, Olfm1, Grik3, and Garba3 were reduced after TRAP in Ng2-CreER::Rpl22-HA mice (FIG. 6J), suggesting a distinct transcriptomic profile of WM cells. PCA analysis distinguishes the input and pulldown fractions from the control and VaD samples into different clusters (FIG. 6K), establishing the validity of the pulldown approach. Differentially expressed genes (DEGs) were identified with an FDR<0.1 (FIG. 6L).

To isolate microglia, a fluorescence-activated cell sorting (FACS) protocol for small WM regions using CX3CR1, CD11b, and CD45 antibodies was applied (FIG. 6M). Based on cell scatter size, microglia obtained from the control brain was designated as P2 (FIG. 6N), while the same fraction and an additional fraction from VaD brain were named P2 and P4 respectively (FIG. 6O). RNA was extracted and sequenced, then clustered in PCA analysis (FIG. 6P). Border-associated macrophage (BAM) genes were traced or undetectable indicating that all FACS sorted cells were pure microglia (FIG. 7L). Thousands of VaD-related microglial DEGs were identified (FIG. 6Q).

Our previous human dataset was derived from male and female VaD patients (63-98 years old, NIH brain bank). The tissues, identified as periventricular WM, were further validated as infarcted using Luxol fast blue staining, making them highly comparable to our mouse model. Most of the VaD-related DEGs were cell type specific, and half of which are dysregulated in only one cell type in both mouse (FIGS. 6R and 6S) and human (FIGS. 6T and 6U). This highlights the importance of studying cell type-specific gene changes to elucidate the mechanisms underlying VaD and to identify connections in cell-specific alterations within the neurovascular unit, as observed in both mouse VaD models and human disease.

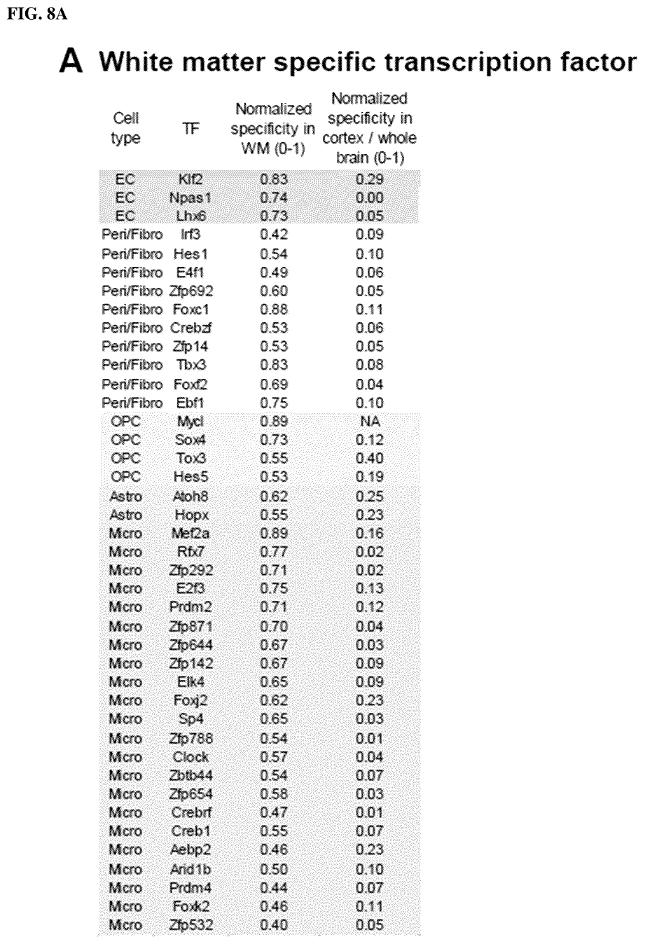

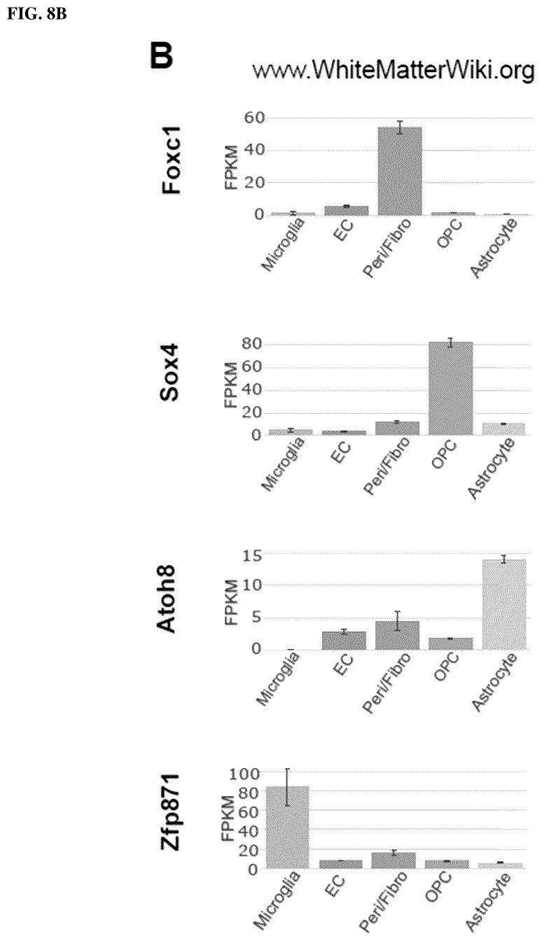

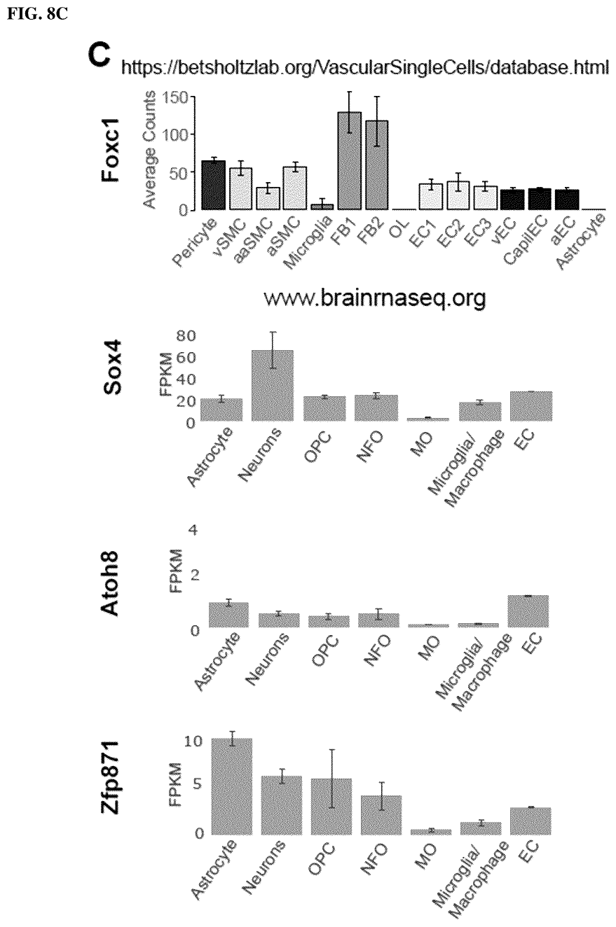

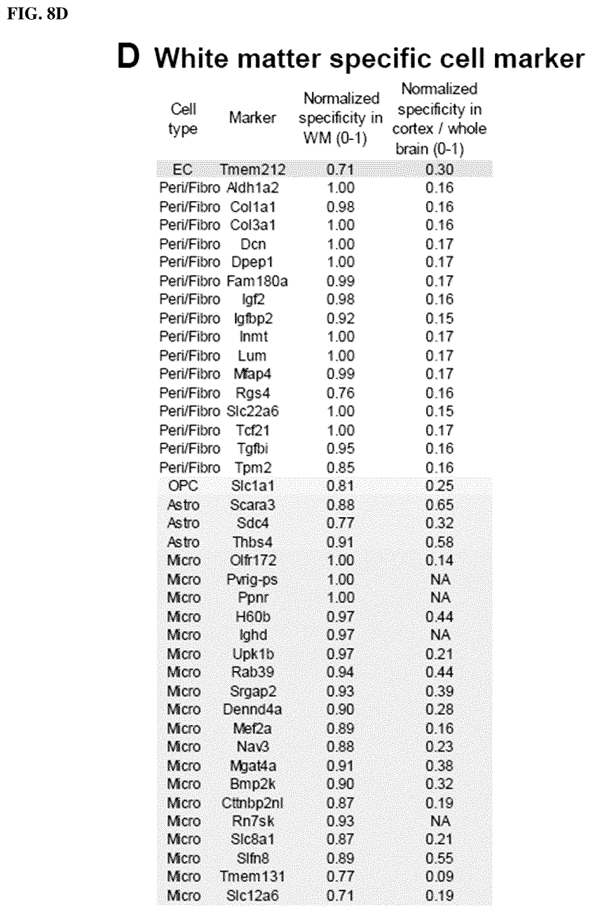

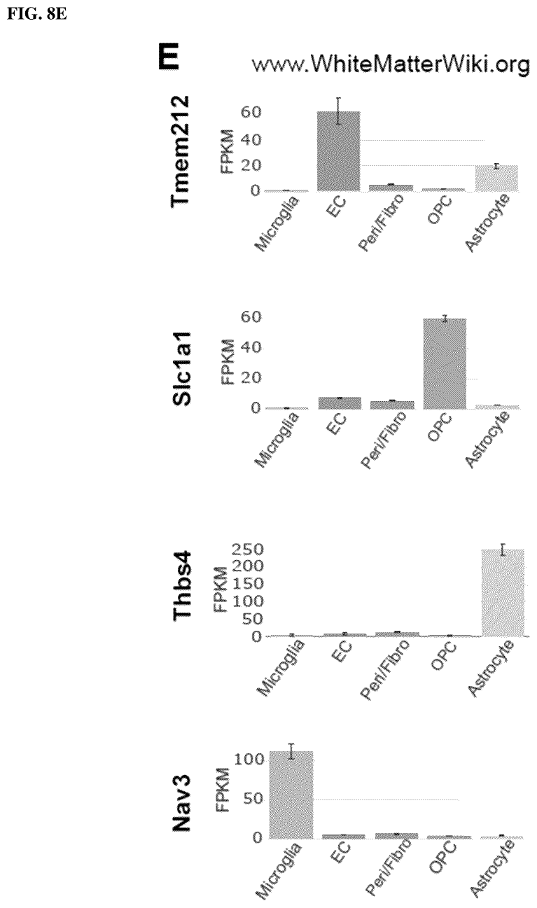

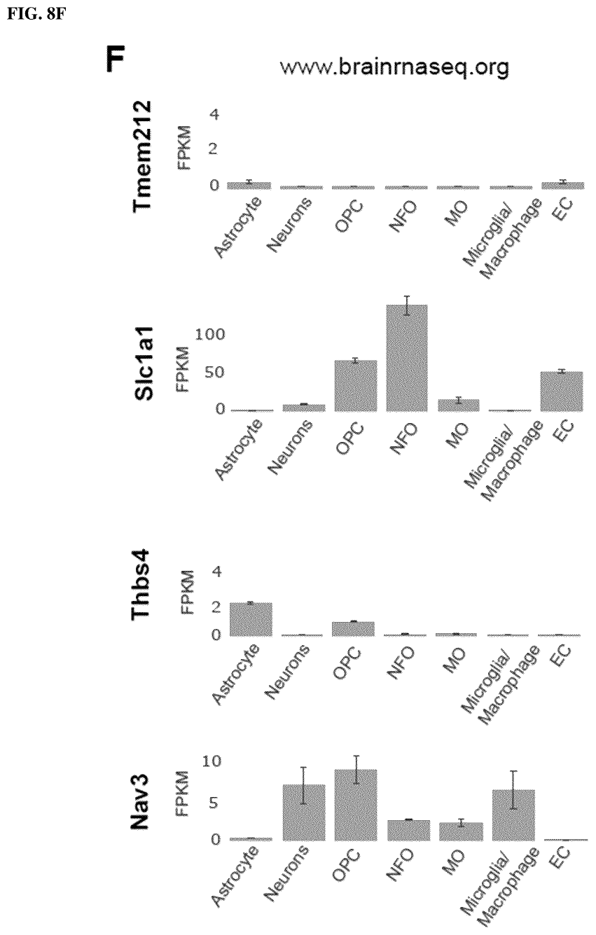

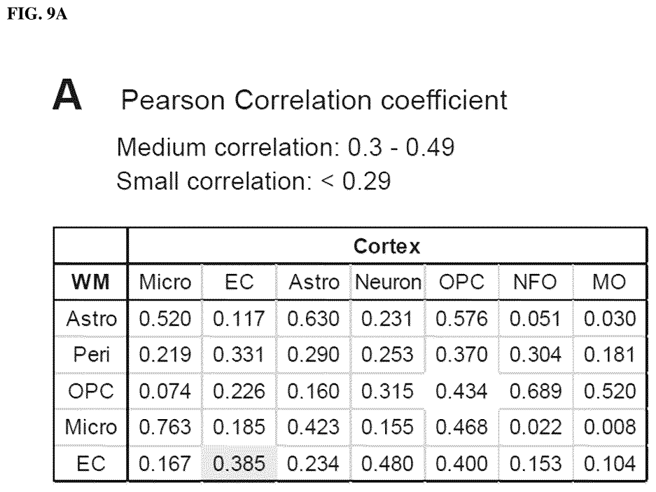

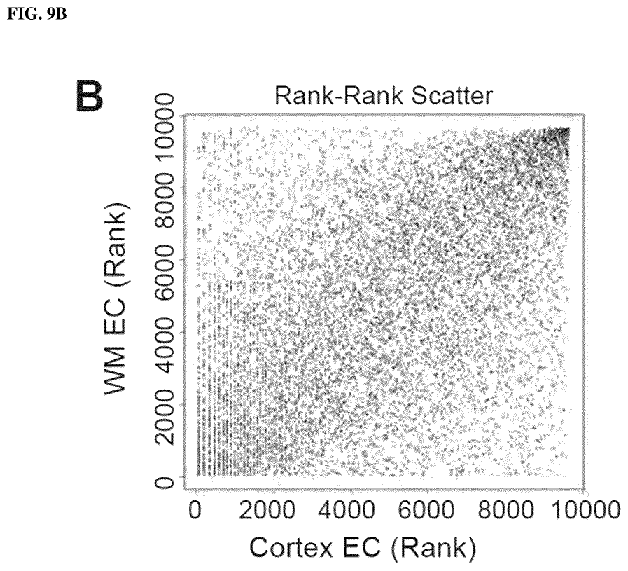

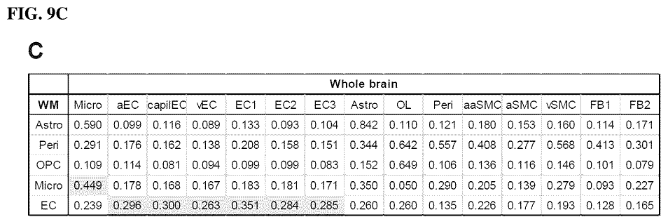

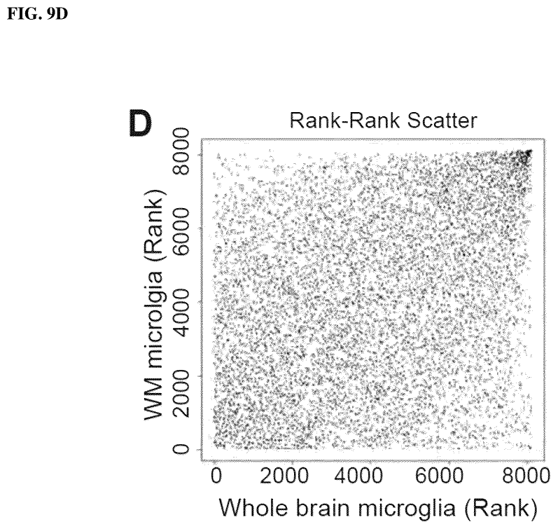

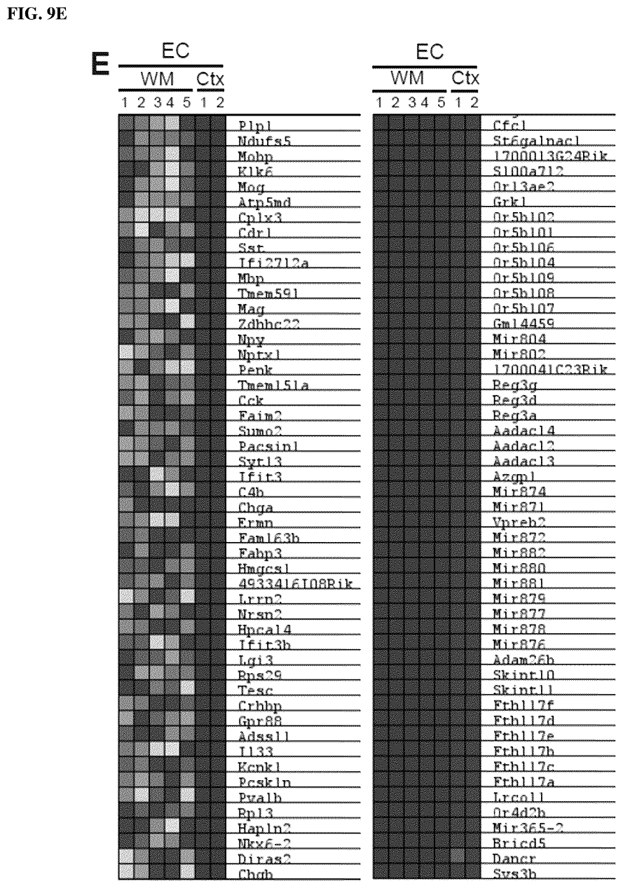

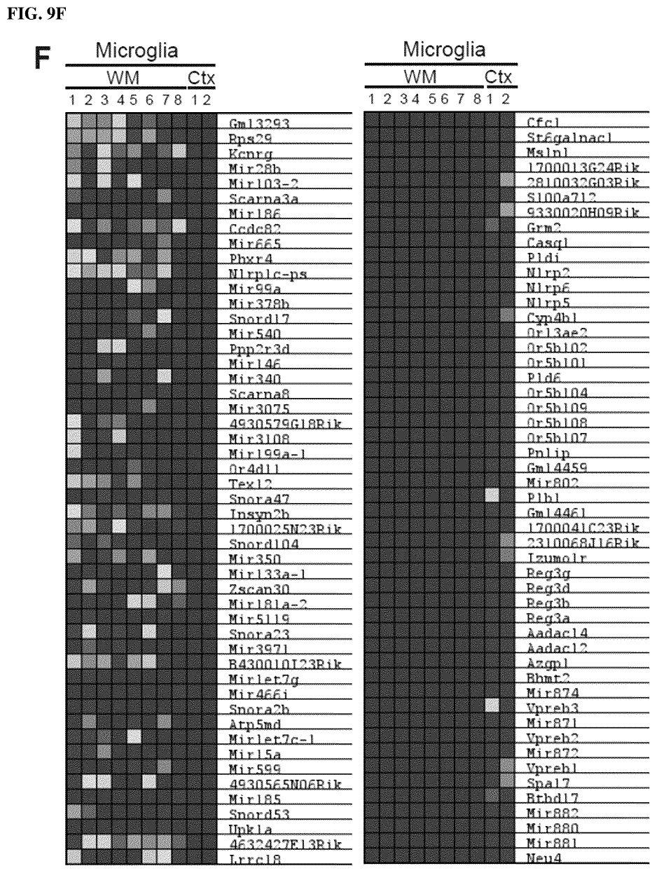

Many cell types have specific transcriptomes in distinct brain regions. A set of cell-type and WM-specific TFs/marker genes were identified (FIG. 8A, Table 2) through comparison to data from developing cortical cells and adult whole brain cells. Foxc1, Sox4, Atoh8, and Zfp871 are examples of distinct WM cell-type specific TFs compared to cortex and whole brain (FIGS. 8B and 8C). TFs are crucial for regulating gene expression. These findings suggest a distinct transcriptional control profile of WM cells against other brain regions. Cell-type marker genes specific for WM were also identified (FIG. 8D, Table 3). Tmem212, Slc1a1, Thbs4, and Nav3 are highly expressed in WM EC, OPC, astrocyte, and microglia respectively, but not in cortical datasets for the same cell types (FIGS. 8E and 8F). Tmem212 is related to cerebral small vessel disease and associated with enlarged periventricular spaces on MRI in aged populations. VaD significantly increased Tmem212 expression in mouse [FPKM, 61.3±10.4 (control) vs 118.5±8.3 (VaD)]. Rank-rank hypergeometric overlap (RRHO) analyses comparing WM cell type transcriptomes with cortical and whole brain show lower similarity of transcription profiles in EC and microglia specifically (FIGS. 9A-9D, Tables 4-6). Furthermore, gene set enrichment analysis (GSEA) showed distinctive gene markers and functional hallmarks of WM EC and microglia compared with the same cell types in cortex (FIGS. 9E and 9F). These results highlight the assembling a WM-specific cell transcriptome as a first step in identifying disease-associated genes in VaD, as well as other WM diseases.

TABLE 2
A partial list of WM specific TFs in 5 cell types

| | | | Normalized |
|---|---|---|---|
| | | | specificity in |
| | | Normalized | cortex/ |
| cell | | specificity in | whole brain |
| type | TF | WM (0-1) | (0-1) |
| EC | Klf2 | 0.83 | 0.29 |
| EC | Npas1 | 0.74 | 0.00 |
| EC | Lhx6 | 0.73 | 0.05 |
| Pericyte | Irf3 | 0.42 | 0.09 |
| Pericyte | Hes1 | 0.54 | 0.10 |
| Pericyte | E4f1 | 0.49 | 0.06 |
| Pericyte | Zfp692 | 0.60 | 0.05 |
| Pericyte | Foxc1 | 0.88 | 0.11 |
| Pericyte | Crebzf | 0.53 | 0.06 |
| Pericyte | Zfp14 | 0.53 | 0.05 |
| Pericyte | Tbx3 | 0.83 | 0.08 |
| Pericyte | Foxf2 | 0.69 | 0.04 |
| Pericyte | Ebf1 | 0.75 | 0.10 |
| OPC | Mycl | 0.89 | NA |
| OPC | Sox4 | 0.73 | 0.12 |
| OPC | Tox3 | 0.55 | 0.40 |
| OPC | Hes5 | 0.53 | 0.19 |
| Astrocyte | Atoh8 | 0.62 | 0.25 |
| Astrocyte | Hopx | 0.55 | 0.23 |
| Microglia | Mef2a | 0.89 | 0.16 |
| Microglia | Rfx7 | 0.77 | 0.02 |
| Microglia | Zfp292 | 0.71 | 0.02 |
| Microglia | E2f3 | 0.75 | 0.13 |
| Microglia | Prdm2 | 0.71 | 0.12 |
| Microglia | Zfp871 | 0.70 | 0.04 |
| Microglia | Zfp644 | 0.67 | 0.03 |
| Microglia | Zfp142 | 0.67 | 0.09 |
| Microglia | Elk4 | 0.65 | 0.09 |
| Microglia | Foxj2 | 0.62 | 0.23 |
| Microglia | Sp4 | 0.65 | 0.03 |
| Microglia | Zfp788 | 0.54 | 0.01 |
| Microglia | Clock | 0.57 | 0.04 |
| Microglia | Zbtb44 | 0.54 | 0.07 |
| Microglia | Zfp654 | 0.58 | 0.03 |
| Microglia | Crebrf | 0.47 | 0.01 |
| Microglia | Creb1 | 0.55 | 0.07 |
| Microglia | Aebp2 | 0.46 | 0.23 |
| Microglia | Arid1b | 0.50 | 0.10 |
| Microglia | Prdm4 | 0.44 | 0.07 |
| Microglia | Foxk2 | 0.46 | 0.11 |
| Microglia | Zfp532 | 0.40 | 0.05 |

TABLE 3
A partial list of WM specific cell type markers

| | | | Normalized |
|---|---|---|---|
| | | Normalized | specificity in |
| | | specificity in | cortex/whole |
| Cell type | Marker | WM (0-1) | brain (0-1) |
| EC | Tmem212 | 0.71 | 0.30 |
| Peri/Fibro | Aldh1a2 | 1.00 | 0.16 |
| Peri/Fibro | Col1a1 | 0.98 | 0.16 |
| Peri/Fibro | Col3a1 | 1.00 | 0.16 |
| Peri/Fibro | Dcn | 1.00 | 0.17 |
| Peri/Fibro | Dpep1 | 1.00 | 0.17 |
| Peri/Fibro | Fam180a | 0.99 | 0.17 |
| Peri/Fibro | Igf2 | 0.98 | 0.16 |
| Peri/Fibro | Igfbp2 | 0.92 | 0.15 |
| Peri/Fibro | Inmt | 1.00 | 0.17 |
| Peri/Fibro | Lum | 1.00 | 0.17 |
| Peri/Fibro | Mfap4 | 0.99 | 0.17 |
| Peri/Fibro | Rgs4 | 0.76 | 0.16 |
| Peri/Fibro | Slc22a6 | 1.00 | 0.15 |
| Peri/Fibro | Tcf21 | 1.00 | 0.17 |
| Peri/Fibro | Tgfbi | 0.95 | 0.16 |
| Peri/Fibro | Tpm2 | 0.85 | 0.16 |
| OPC | Slc1a1 | 0.81 | 0.25 |
| Astrocyte | Scara3 | 0.88 | 0.65 |
| Astrocyte | Sdc4 | 0.77 | 0.32 |
| Astrocyte | Thbs4 | 0.91 | 0.58 |
| Micro | Olfr172 | 1.00 | 0.14 |
| Micro | Pvrig-ps | 1.00 | NA |
| Micro | Ppnr | 1.00 | NA |
| Micro | H60b | 0.97 | 0.44 |
| Micro | Ighd | 0.97 | NA |
| Micro | Upk1b | 0.97 | 0.21 |
| Micro | Rab39 | 0.94 | 0.44 |
| Micro | Srgap2 | 0.93 | 0.39 |
| Micro | Dennd4a | 0.90 | 0.28 |
| Micro | Mef2a | 0.89 | 0.16 |
| Micro | Nav3 | 0.88 | 0.23 |
| Micro | Mgat4a | 0.91 | 0.38 |
| Micro | Bmp2k | 0.90 | 0.32 |
| Micro | Cttnbp2nl | 0.87 | 0.19 |
| Micro | Rn7sk | 0.93 | NA |
| Micro | Slc8a1 | 0.87 | 0.21 |
| Micro | Slfn8 | 0.89 | 0.55 |
| Micro | Tmem131 | 0.77 | 0.09 |
| Micro | Slc12a6 | 0.71 | 0.19 |

TABLE 4
Pearson correlation coefficient comparing
WM with cortex cells using RRHO

| | Cortex | | | | | | |
|---|---|---|---|---|---|---|---|
| WM | Micro | EC | Astro | Neuron | OPC | NFO | MO |
| Astro | 0.520 | 0.117 | 0.630 | 0.231 | 0.576 | 0.051 | 0.030 |
| Peri | 0.219 | 0.331 | 0.290 | 0.253 | 0.370 | 0.304 | 0.181 |
| OPC | 0.074 | 0.226 | 0.160 | 0.315 | 0.434 | 0.689 | 0.520 |
| Micro | 0.763 | 0.185 | 0.423 | 0.155 | 0.468 | 0.022 | 0.008 |
| EC | 0.167 | 0.385 | 0.234 | 0.480 | 0.400 | 0.153 | 0.104 |

TABLE 5
Pearson correlation coefficient comparing
WM with whole brain cells using RRHO

| | Whole brain | | | | | | |
|---|---|---|---|---|---|---|---|
| WM | Micro | aEC | capilEC | vEC | EC1 | EC2 | EC3 |
| Astro | 0.590 | 0.099 | 0.116 | 0.089 | 0.133 | 0.093 | 0.104 |
| Peri | 0.291 | 0.176 | 0.162 | 0.138 | 0.208 | 0.158 | 0.151 |
| OPC | 0.109 | 0.114 | 0.081 | 0.094 | 0.099 | 0.099 | 0.083 |
| Micro | 0.449 | 0.178 | 0.168 | 0.167 | 0.183 | 0.181 | 0.171 |
| EC | 0.239 | 0.296 | 0.300 | 0.263 | 0.351 | 0.284 | 0.285 |

TABLE 6
Pearson correlation coefficient comparing
WM with whole brain cells using RRHO

| | Whole brain | | | | | | | |
|---|---|---|---|---|---|---|---|---|
| WM | Astro | OL | Peri | aaSMC | aSMC | vSMC | FB1 | FB2 |
| Astro | 0.842 | 0.110 | 0.121 | 0.180 | 0.153 | 0.160 | 0.114 | 0.171 |
| Peri | 0.344 | 0.642 | 0.557 | 0.408 | 0.277 | 0.568 | 0.413 | 0.301 |
| OPC | 0.152 | 0.649 | 0.106 | 0.136 | 0.116 | 0.146 | 0.101 | 0.079 |
| Micro | 0.350 | 0.050 | 0.290 | 0.205 | 0.139 | 0.279 | 0.093 | 0.227 |
| EC | 0.260 | 0.260 | 0.135 | 0.226 | 0.177 | 0.193 | 0.128 | 0.165 |

### Example 10: VaD Modeling in Mouse Brain Drives Cells Towards Aging

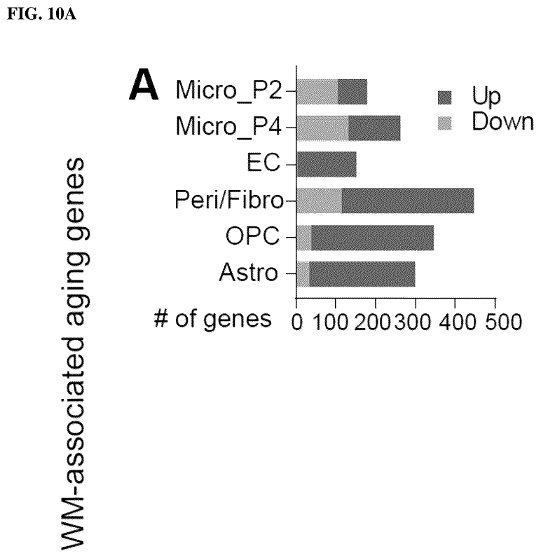

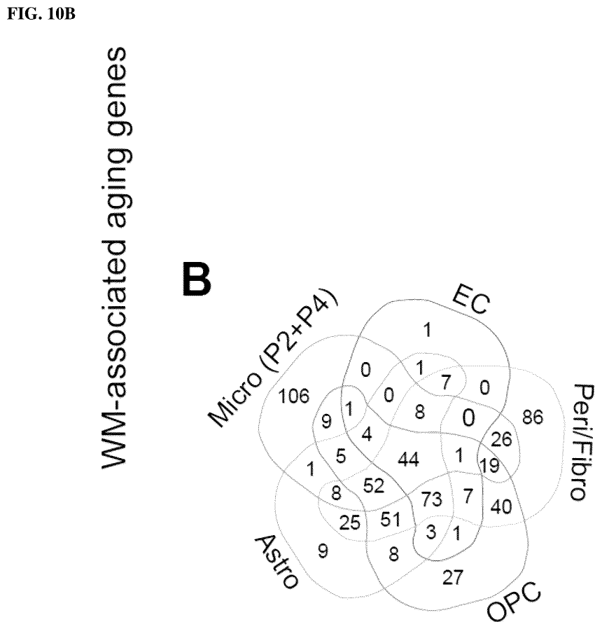

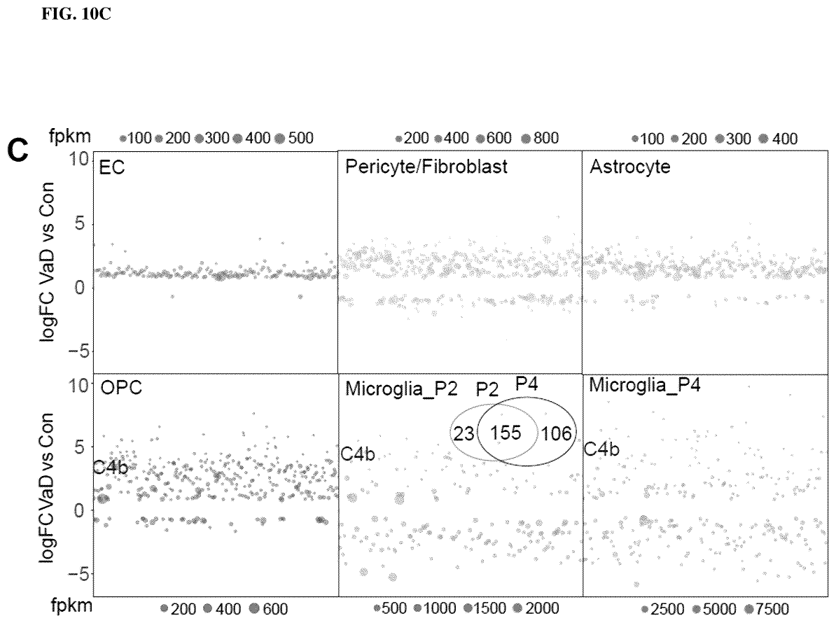

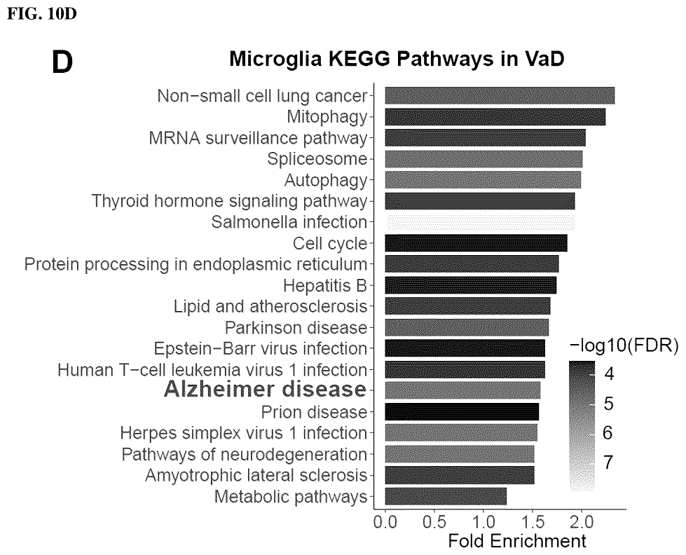

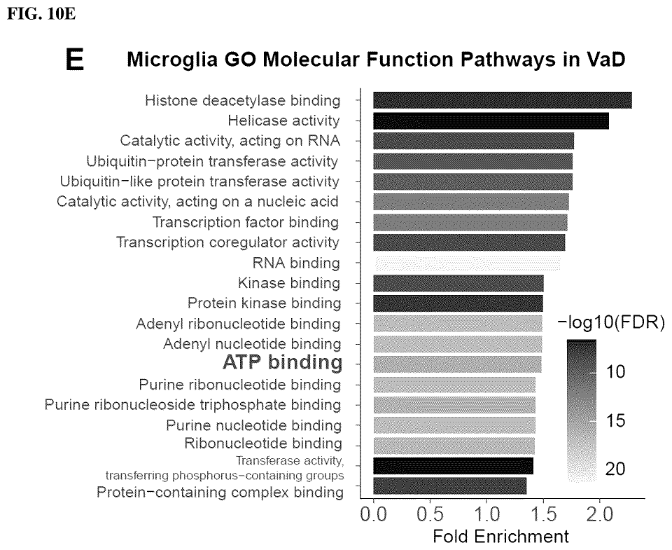

Glial aging is particularly accelerated in WM compared with cortical regions, and the corpus callosum shows the most profound and earliest shifts of genes in an aging profile. Substantial numbers of WM-associated aging genes were differentially expressed in 5 types of cells (FIG. 10A), mostly up-regulated, indicating that the VaD model in the young adult mouse caused a molecular expression profile that is seen in aging. Microglia exhibited the most unique shift of WM-associated aging genes (FIG. 10B), and most substantial fold change in VaD (FIG. 10C). Notably, the cluster of P4 microglia, exhibited a larger number and greater change of WM-associated aging genes compared to the P2-cluster microglia (FIG. 10C). A key WM-associated aging gene, C4b, which is a complement component and major schizophrenia risk factor, is specifically up-regulated in OPC and microglia in VaD (FIG. 10C). Function analysis with KEGG pathways showed that microglial transcriptomic changes in the VaD model were associated with dysregulation of major molecular pathways, including those involved in Alzheimer's disease (FIG. 10D). GO enrichment analysis also indicated that microglia in VaD model were involved in dysregulated molecular functional pathways including ATP binding (FIG. 10E).

### Example 11: Mapping the Intercellular Interactome in VaD

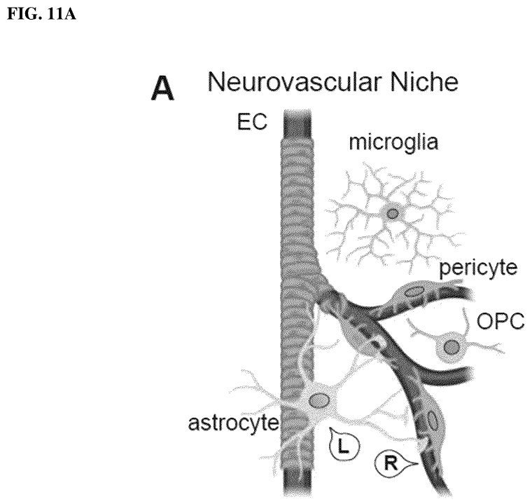

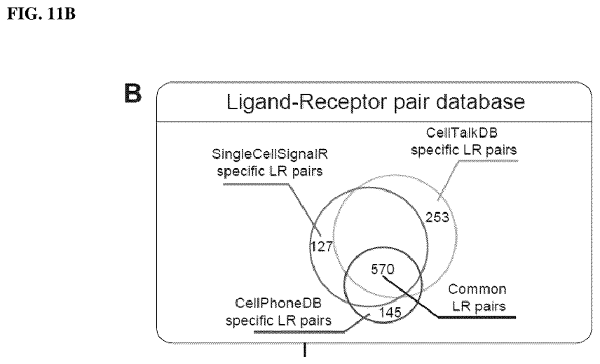

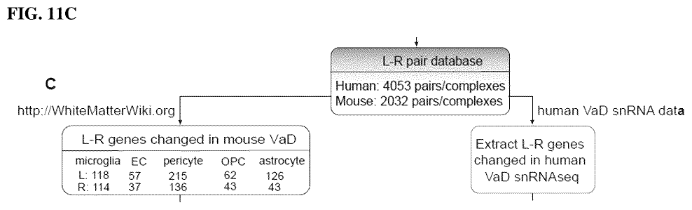

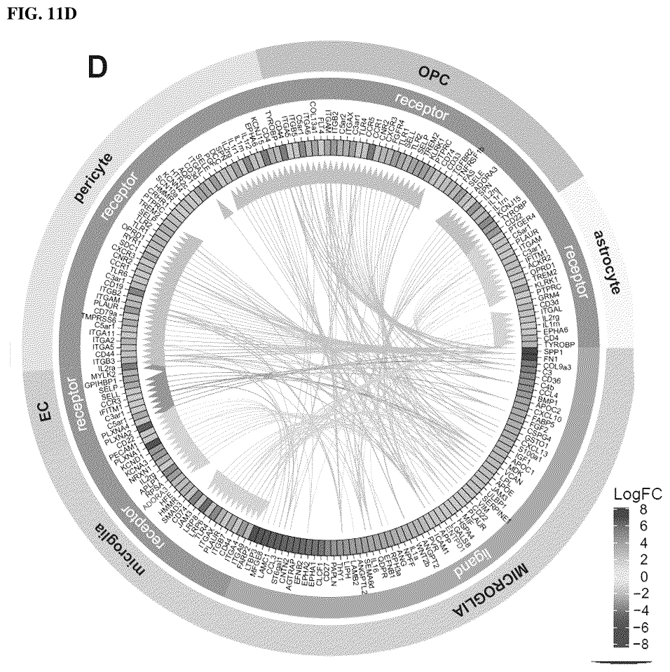

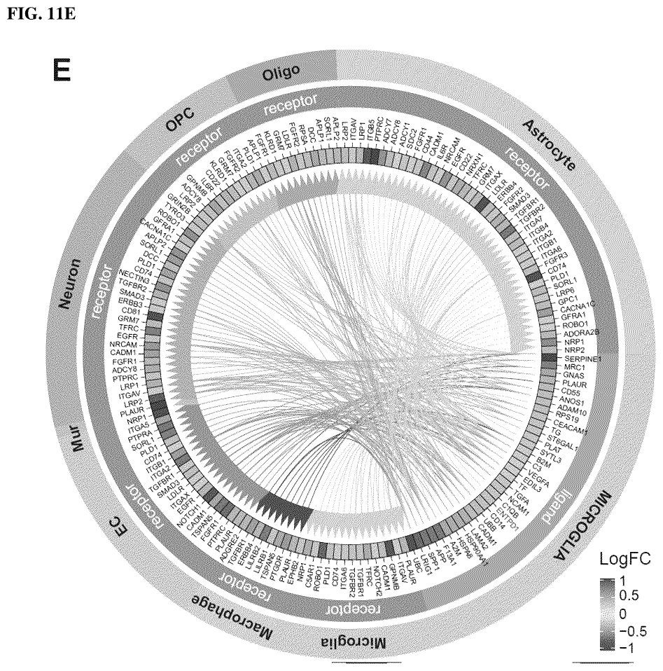

To determine the cell-cell signaling systems within the WM neurovascular niche (FIG. 11A), the VaD cell-type specific transcriptomes were analyzed for ligand-receptor (L-R) systems that are conjointly and differentially regulated. First, a L-R database was assembled by merging three major L-R libraries covering 4053 human and 2032 mouse L-R pairs/complexes (FIG. 11B), which is currently the largest of its kind. Subsequently, the number of ligand and receptor genes related to mouse VaD were identified in each cell type. For example, from the microglia RNA-Seq data in mouse VaD, 118 ligands and 114 receptors were identified (FIG. 11C). Human VaD related L-R genes were also identified from our published snRNA-Seq dataset.

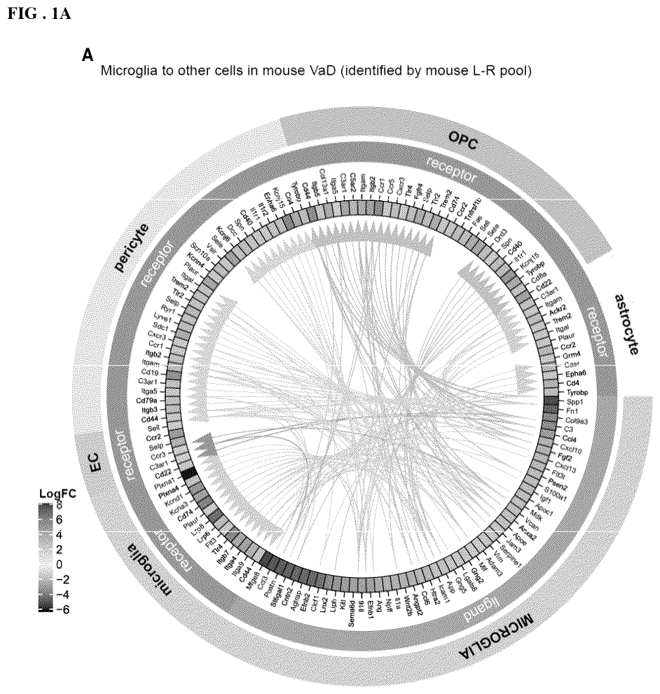

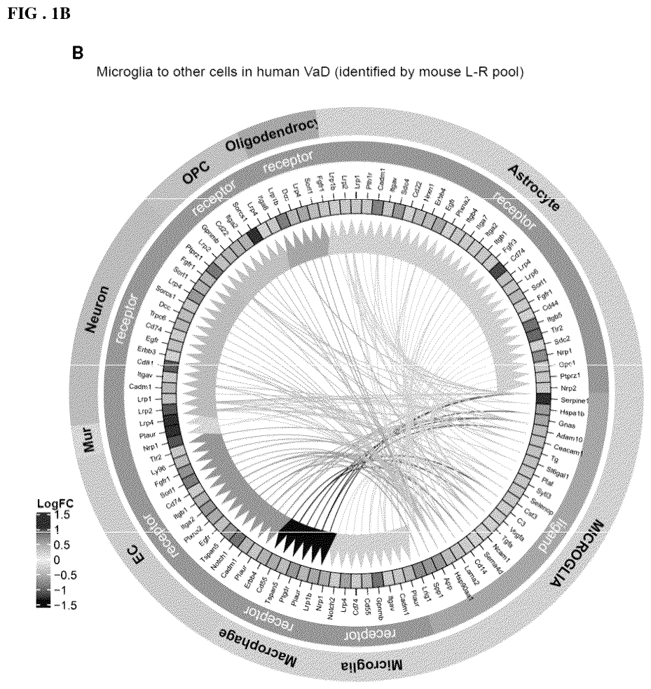

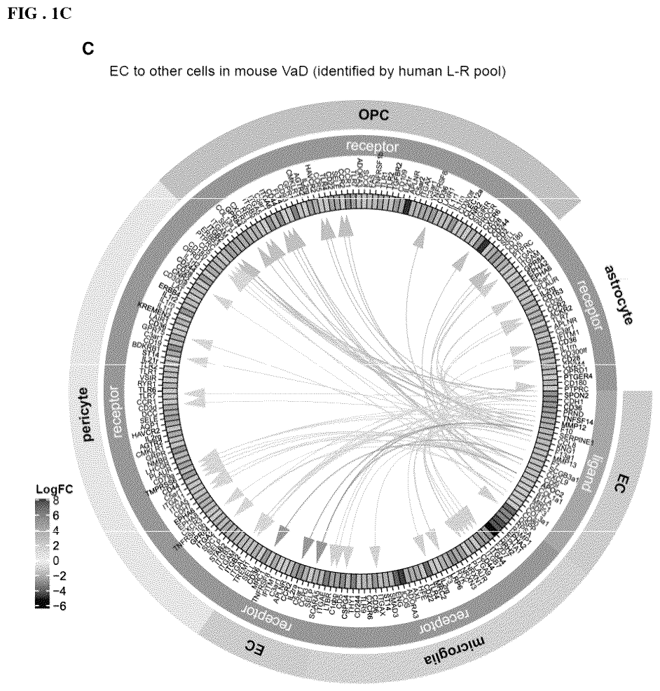

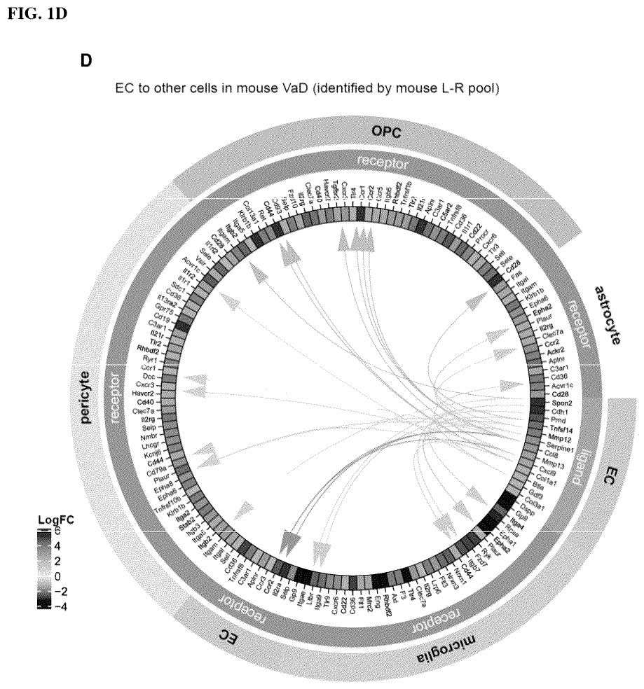

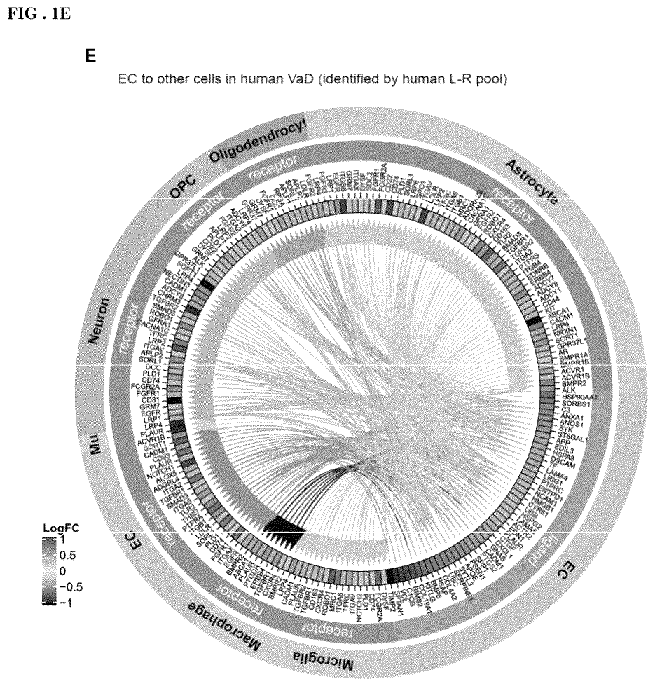

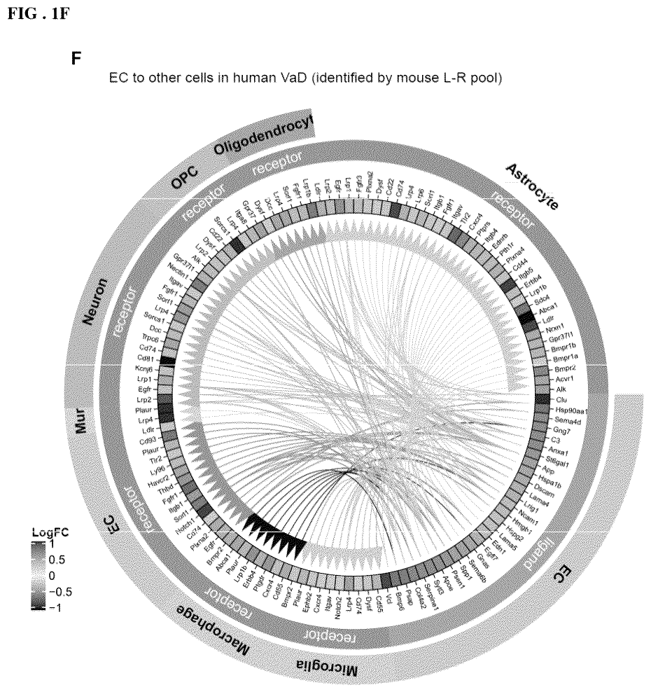

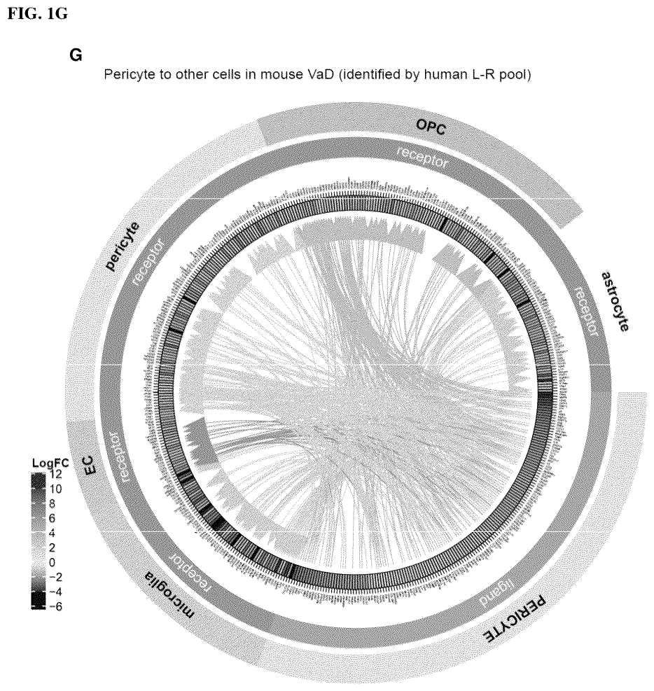

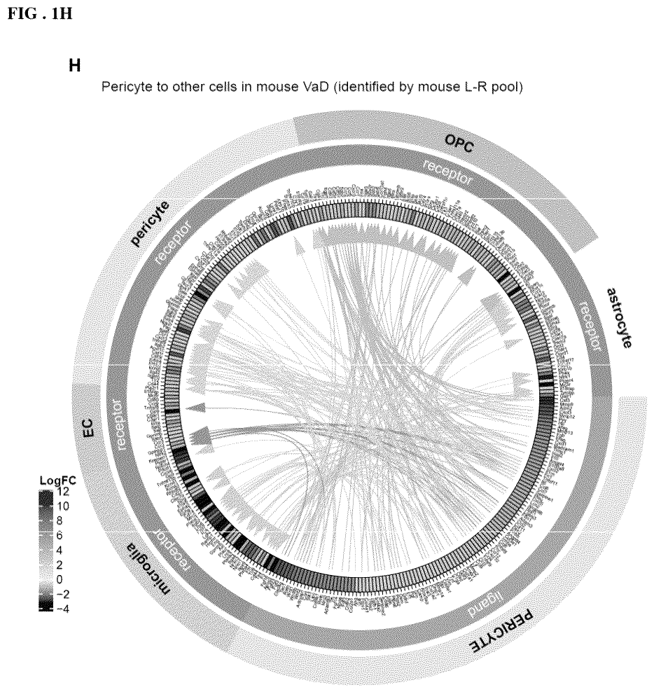

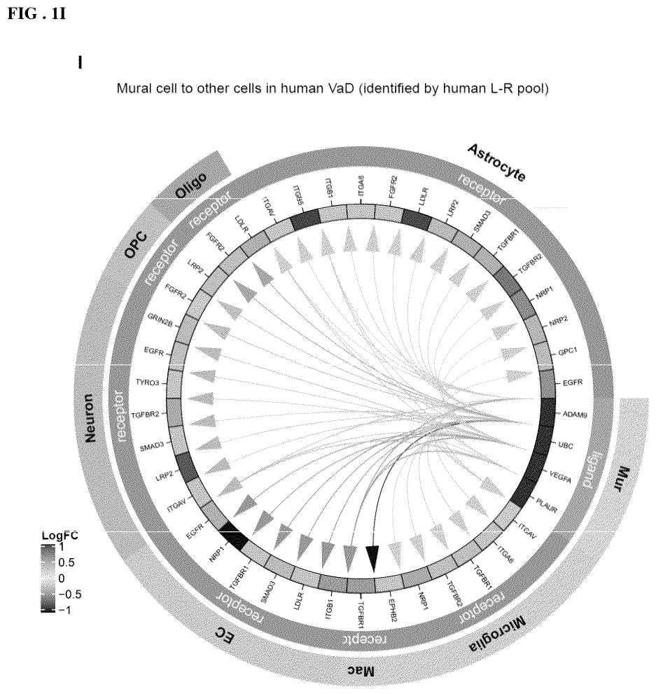

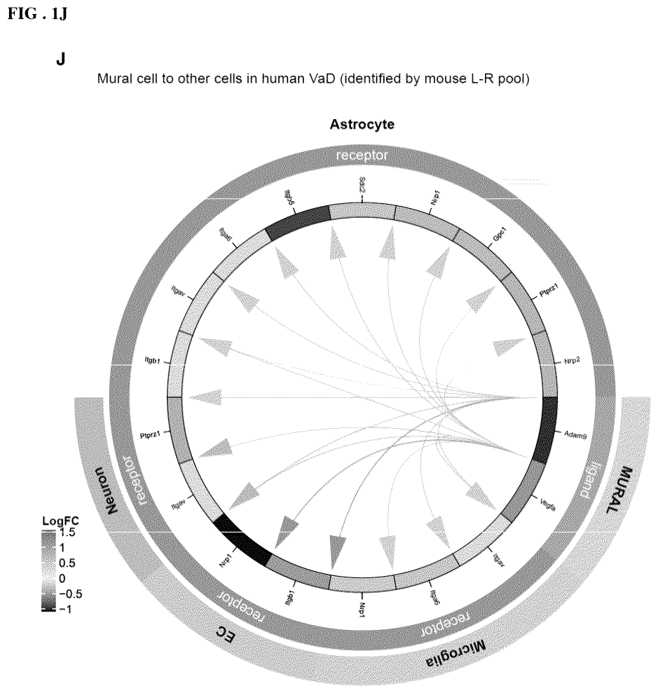

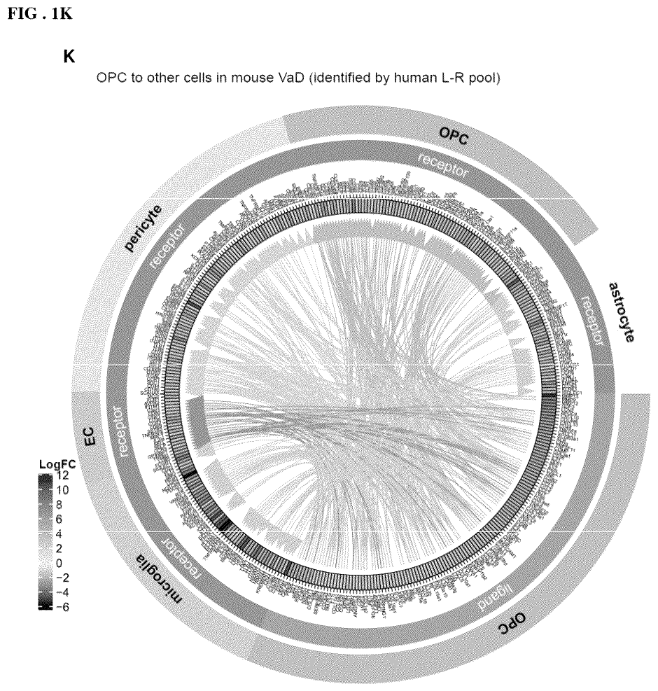

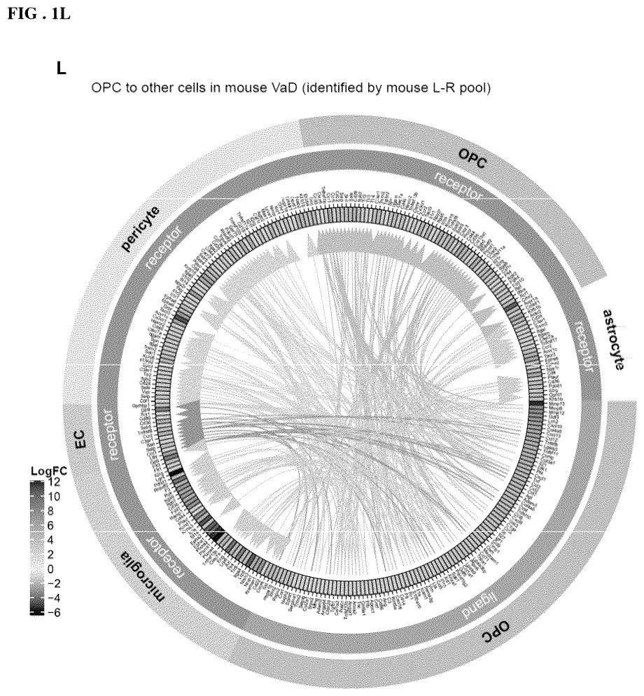

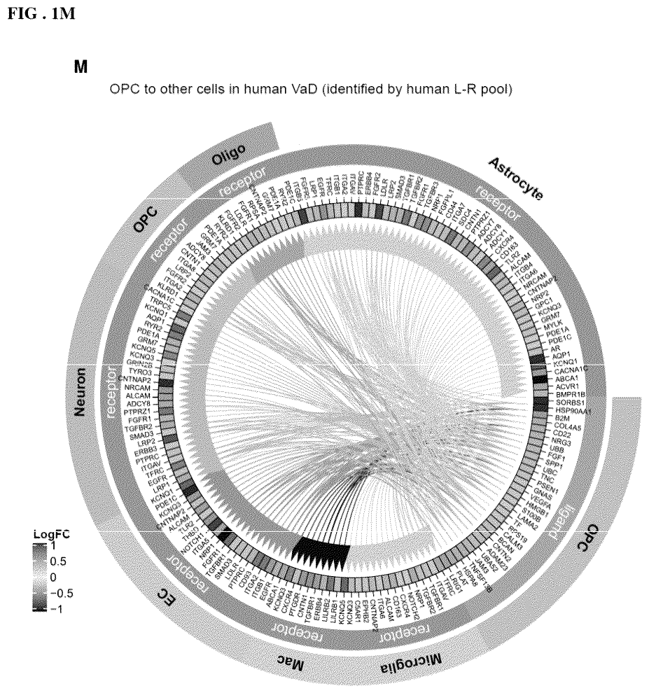

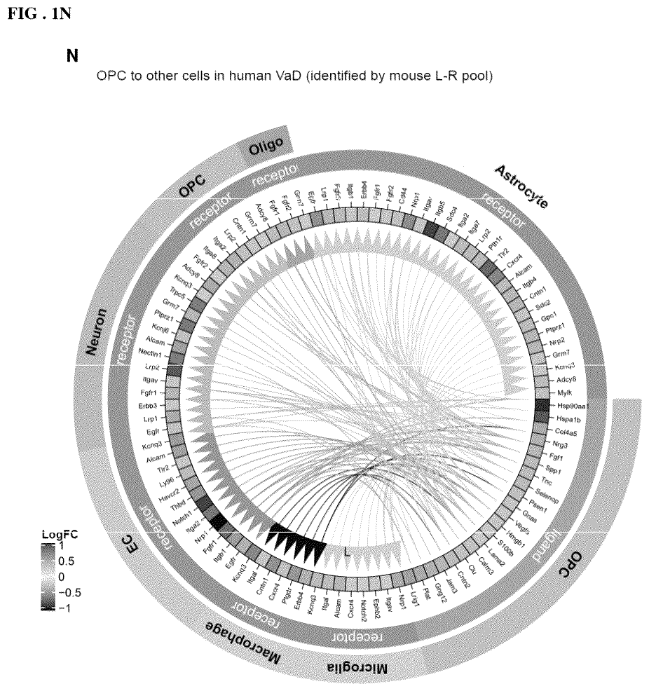

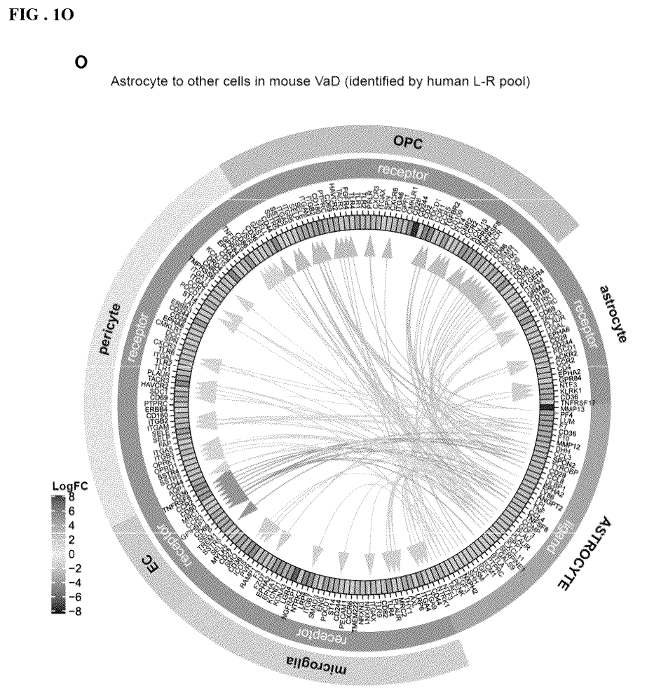

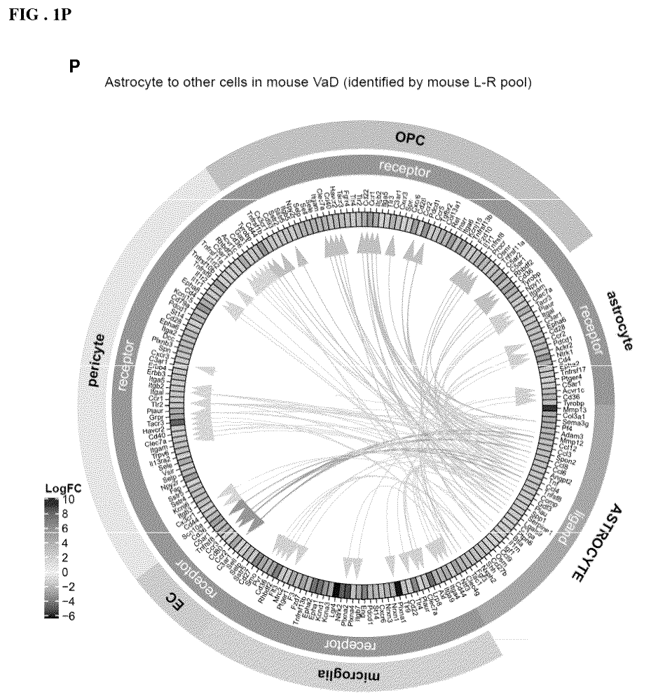

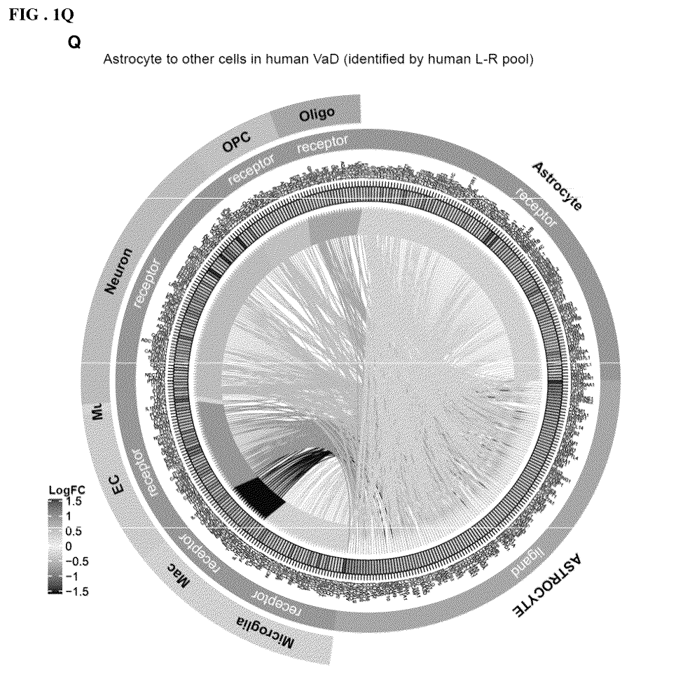

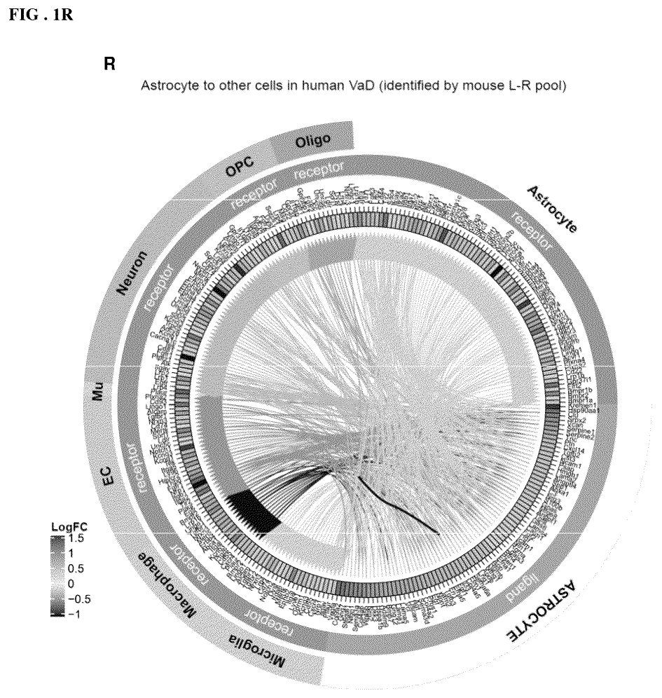

To determine the intercellular interactome during VaD recovery, cells were linked together via L-R interactions, which can be visually presented by Circos plots (FIGS. 1A-1R, 11D and 11E). In mouse VaD, the top microglial ligands (LogFC>2 or <−1.5) identified through interfacing the human L-R pool, were connected towards corresponding receptors in other cell types, where SPP1 was the most increased ligand, and FARP2 was the most decreased ligand (FIG. 11D, Table 4); Certain L-R pairs were excluded from this circos plot due to the use of stringent thresholds for log FC and FDR values to accommodate the page size limitations. In human VaD, the microglial ligands were identified interfacing the human L-R pool and connected towards corresponding receptors in other cell types (FIG. 11E, Table 5).

### Example 12: Categorization of L-R Genes as Potential Candidates for VaD Treatment

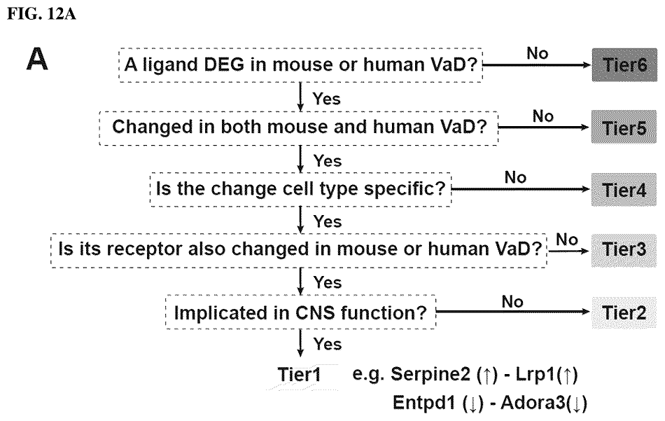

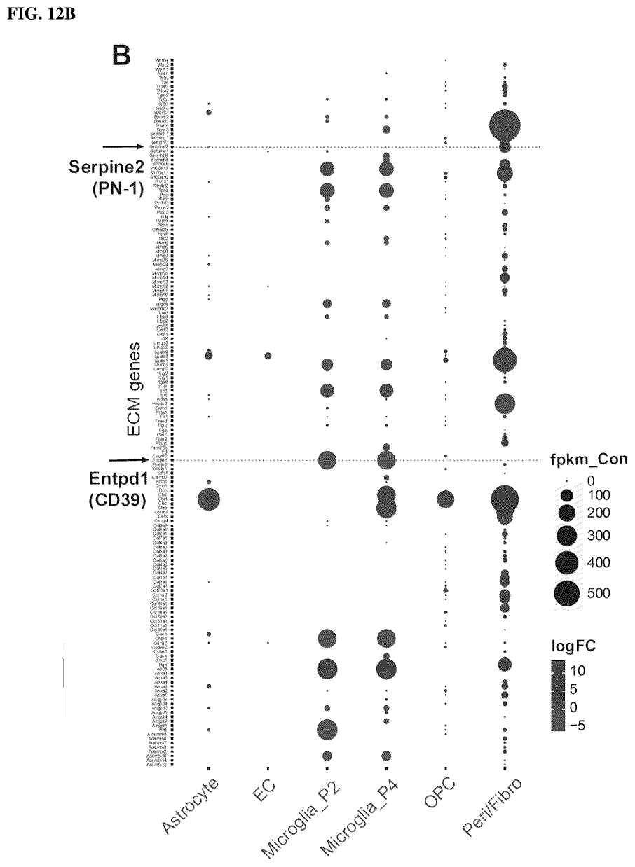

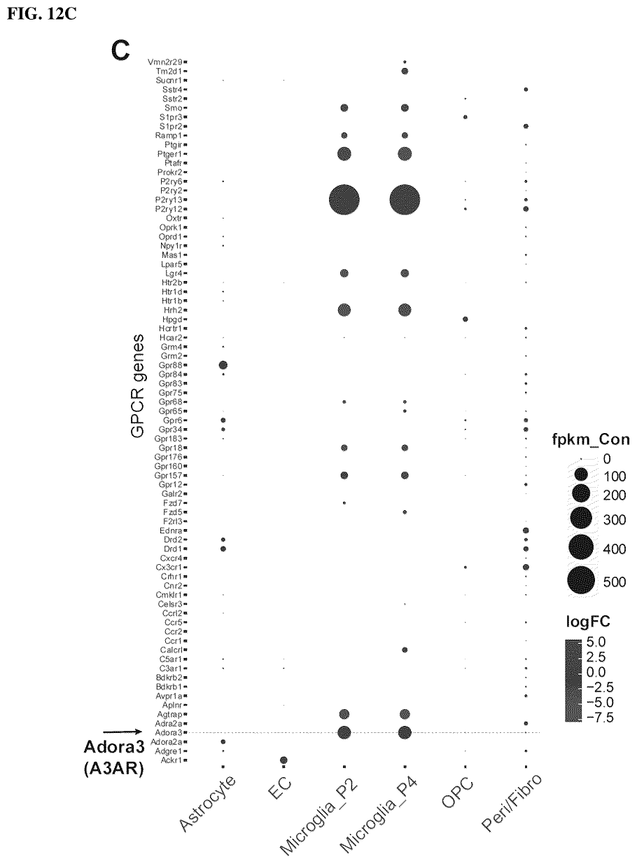

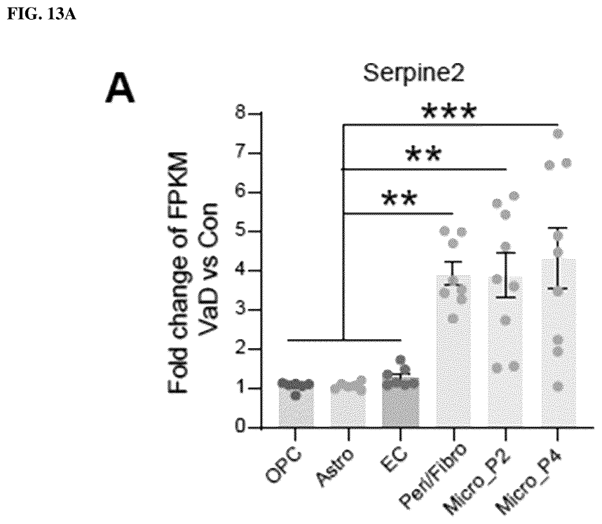

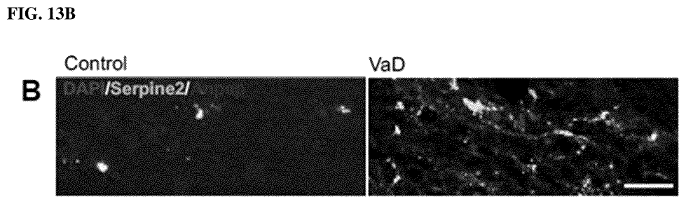

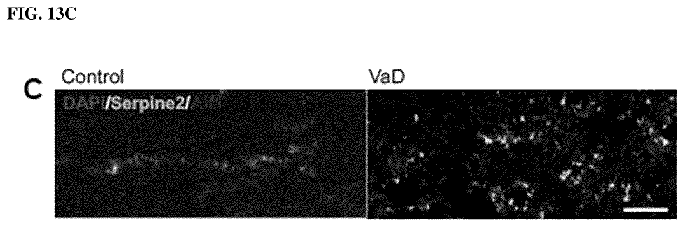

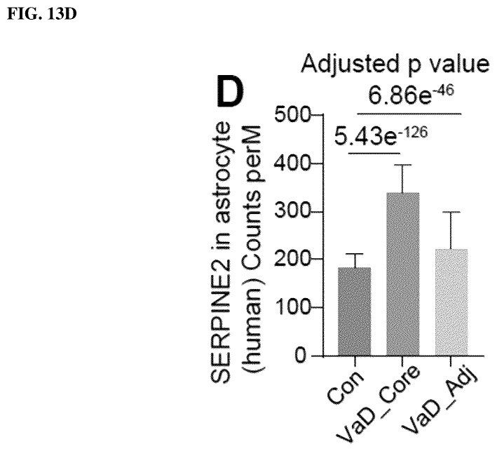

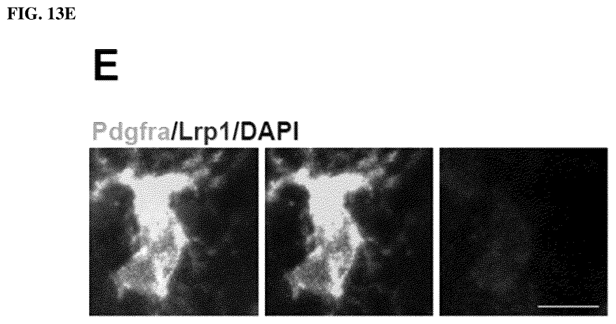

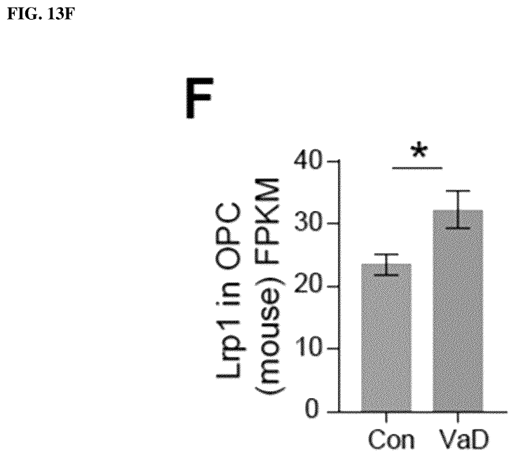

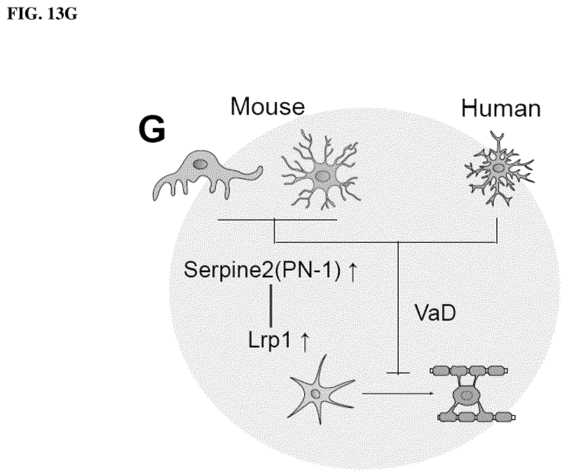

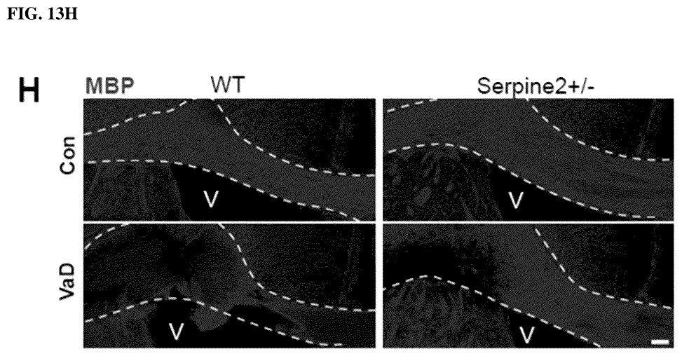

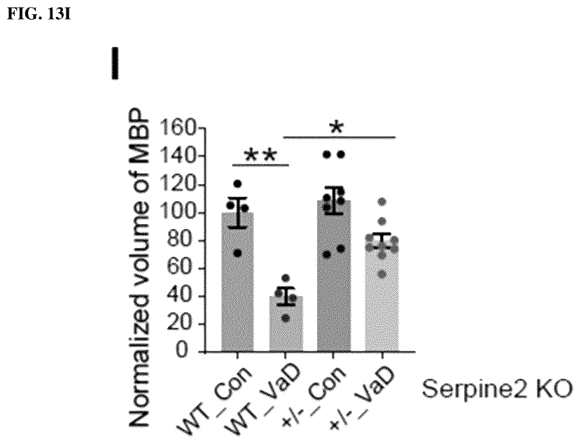

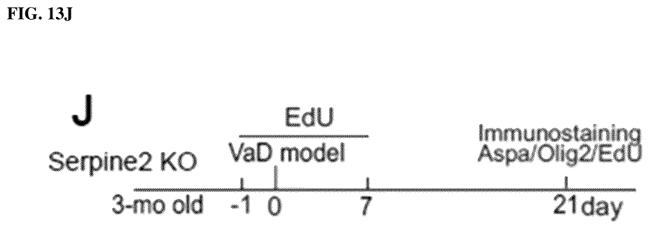

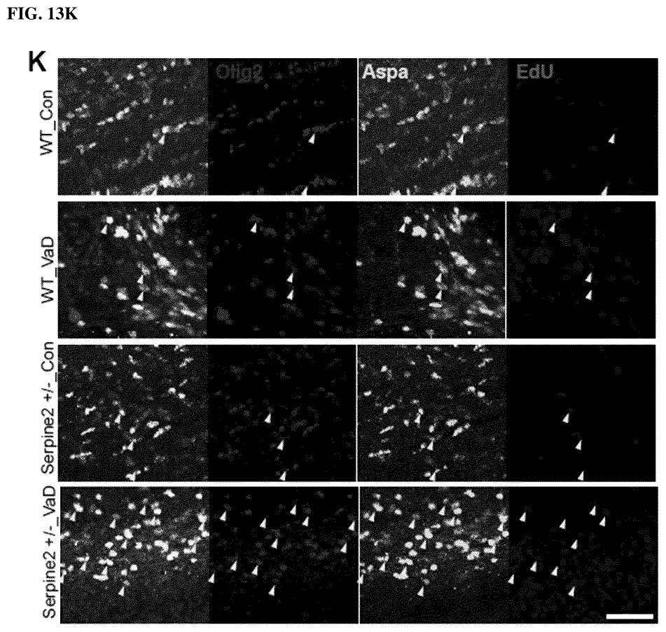

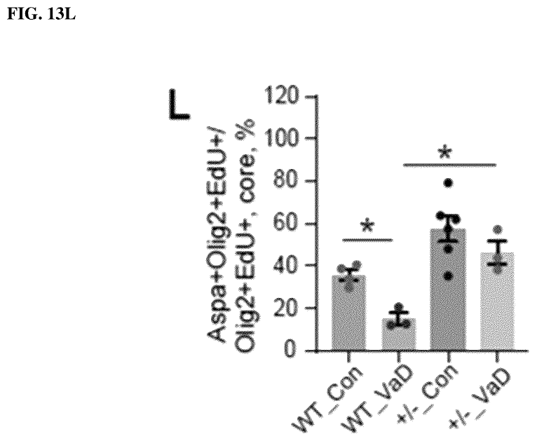

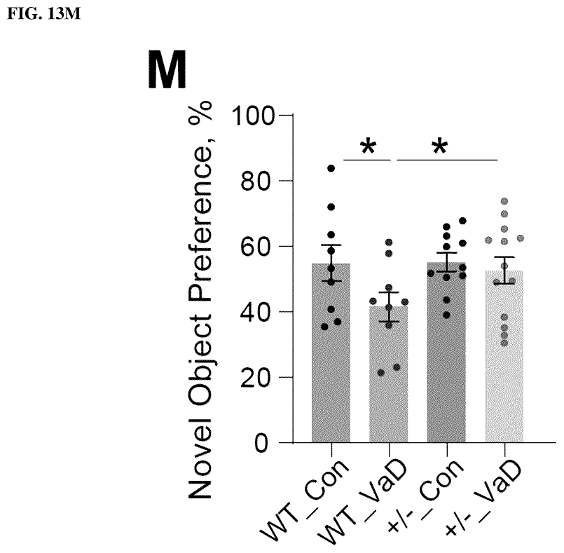

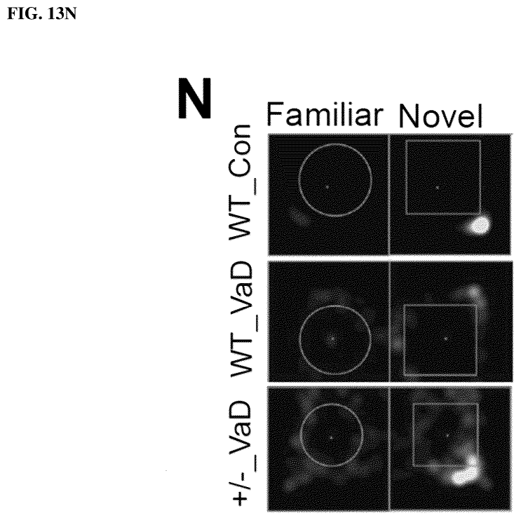

In order to screen potential targets for VaD treatments, a series of molecular identity and significance criteria was used to categorize the DEGs in ascending order of priority for study (FIG. 12A). First, the candidate may be a ligand (Tier 5). Otherwise, the DEG is categorized as Tier 6. Second, the ligand candidates from the mouse VaD dataset had to also be significantly regulated in human VaD (Tier 4). Next, ligand candidates comparably changed in only one or two cell types were prioritized (Tier 3). Then, those with significant change in corresponding receptors were identified (Tier 2). Tier 6 to Tier 3 categorization was done through bioinformatic approaches. Finally, the candidates with a reported function related to neurological activity were classified as Tier 1, otherwise, classified as Tier 2. In this selection scheme, Tier 1 L-R genes are attractive candidates under the scope of an intercellular interactome for VaD study. Among Tier 1 candidates, Serpine2-Lrp1 and CD39-A3AR intercellular signaling pathways were selected for mechanistic investigation and functional analyses to validate the translatability of therapeutic targets identified for VaD treatment, further discussed in FIGS. 13-17.

To classify the L-R candidates in terms of functions, extracellular matrix (ECM) and G-protein coupled-receptor (GPCR) were identified as two major groups of candidates (FIGS. 12B and 12C). Microglia and pericyte/fibroblast are more involved in the regulation of ECM components, including the Tier 1 candidates: ECM regulating gene Entpd1 (CD39) and Serpine2 (PN-1) (FIG. 12B). Microglial Serpine2 was filtered out because a more stringent threshold (FDR<0.005) was used for microglia to reduce the complexity of the bubble plot, ensuring better readability. A recent study also reported that pericyte and fibroblast play important roles in the ECM component after stroke. Microglia also exhibited higher dysregulation of GPCRs, including the Tier 1 receptor: Adora3 (A3AR), a receptor in the CD39 pathway (FIG. 12C).

### Example 13: Serpine2-Lrp1 Intercellular Pathway Inhibits OPC Maturation in VaD Tissue Repair

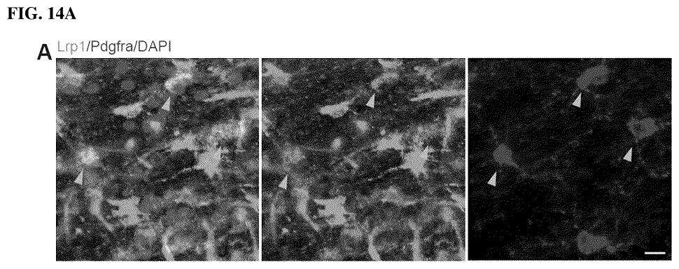

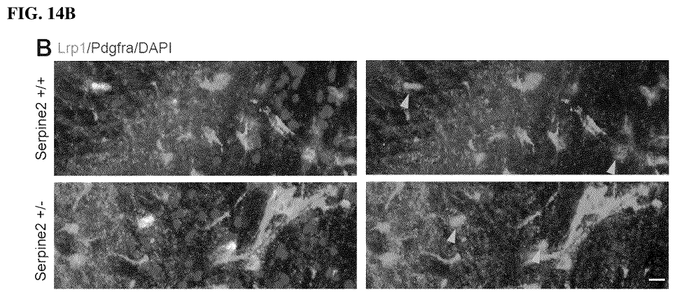

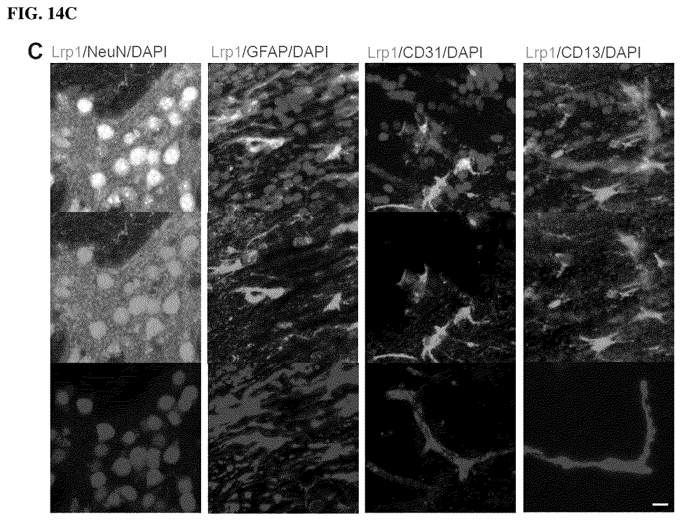

Serpine2 was significantly increased in pericytes/fibroblasts and microglia in mouse VaD (FIGS. 13A-13C), and astrocytes in human VaD (FIG. 13D). Serpine2-encoded protease nexin-1 (PN-1) plays a role in multiple sclerosis and Alzheimer's disease. It binds to low density lipoprotein receptor-related protein 1 (Lrp1) to mediate functions in cancer and vascular biology. Lrp1 is also implicated in Alzheimer's disease. Lrp1 expression in OPC is higher than in myelinated oligodendrocytes. Its expression in OPC was further validated using immunostaining (FIGS. 13E and 14A), which is similar in wild type and Serpine2 deficient animals (FIG. 14B). Lrp1 is also expressed in neurons, astrocytes, but not EC and pericytes/fibroblast in WM (FIG. 14C). RNA-Seq showed a significant increase of Lrp1 expression in OPC during VaD (FIG. 13F). Therefore, the Serpine2-Lrp1 axis was overall up-regulated and may play a role in the differentiation of OPCs into myelinating oligodendrocytes which is required for VaD tissue repair (FIG. 13G). To investigate the function of Serpine2 on OPC differentiation, remyelination was evaluated in Serpine2 KO mice during VaD recovery. Homozygous knockouts were excluded, considering their significant epileptic activity and impairment of memory associated-long term potentiation (LTP). Immunostaining of MBP showed that the reduction of myelin volume in VaD was ameliorated by reduced Serpine2 expression in heterozygous siblings (FIGS. 13H and 13I). Quantification of myelinating oligodendrocytes (Aspa+Olig2+EdU+) differentiated from newly formed OPCs (Olig2+EdU+) in the infarct core indicated that the differentiation of newborn OPCs during VaD repair was increased by Serpine2 deficiency (FIGS. 13J-13L). Strikingly, the NOR task showed that reduced Serpine2 expression rescued the memory deficit in VaD (FIGS. 13M and 13N). To summarize, Serpine2-Lrp1 axis is elevated in VaD, and Serpine2 reduction promotes OPC responses toward myelination and restores memory function in VaD model.

### Example 14: CD39-A3AR Intercellular Pathway is a Potential Target for Tissue and Behavior Repair in VaD

CD39 (Entpd1) is an ectonucleotidase that plays a significant role in regulating the balance of extracellular ATP. It has potential as a cancer immunotherapeutic target. Blockade of CD39 may prevent cancer cell adhesion to the ECM. Neuron-microglia interaction through CD39-A1AR (Adenosine A1 receptor) is implicated in negative feedback control of neuronal activity. Cell type-specific RNA-Seq shows that CD39, and its receptor A3AR (Adenosine A3 receptor, gene name Adora3) were specifically expressed in microglia, and significantly reduced in VaD (FIGS. 16A and 16B). A1AR is not significantly changed in VaD or specifically expressed in one or two cell types (FIG. 16E). Extracellular signaling in CD39-A3AR occurs as ATP is converted by CD39 to ADP and AMP; AMP is converted by CD73 (Nt5e) to adenosine; adenosine activates A3AR and is degraded by adenosine deaminase (Ada). RNA-Seq data indicated low expression of Nt5e (FPKM≤3), and no significant change in Nt5e and Ada (FIGS. 16F and 16G). These data position extracellular ATP and its regulation by CD39 as a potential therapeutic target in VaD.

The cellular localization and protein levels of CD39 were determined in mouse and human, and as a function of age and VaD. Co-staining of CD39 with cell specific markers show that CD39 was specifically expressed in microglia and EC (FIG. 15A). Aging alone (3-mo to 30-mo in the mouse) did not lead to reduction of CD39 expression, while aging and VaD together significantly reduced CD39 expression in EC and microglia (FIG. 15B). Human VaD snRNA-Seq showed that microglial and endothelial CD39 expression in the WM lesion core and adjacent area was significantly reduced (FIGS. 16C and 16D, derived from). Immunohistochemistry (FIG. 15C) further validated a significant reduction (50.6%) of CD39 expression in VaD brains (FIG. 15D). In contrast, the immunoreactivity of Glut1 (EC marker) was reduced only by 20.3%, which may be due to ischemic injury in vessels, while Iba1 (microglia marker) was not significantly changed (FIGS. 16H-16K). Therefore, reduced CD39 immunoreactivity in human VaD is caused by its impaired expression in EC and microglia, including some decreased density of blood vessels. Notably, the CD39 expression was inversely related to age in human VaD (FIG. 15E), which indicates that aging and VaD synergistically reduce CD39 expression in human, like in the mouse. Immunostaining validated the expression of A3AR in WM microglia (FIG. 16L) and the reduction of Adora3 in microglia was validated by RNAScope (FIGS. 16M and 16N).

These data may suggest that intercellular CD39-A3AR signaling is an endogenous mechanism associated with VaD: Endothelial and microglial CD39 enhance the conversion of ATP into adenosine, which modulates microglia through A3AR; Infarct and aging together reduce CD39 expression in EC and microglia during VaD, which lead to impaired signaling to microglia in the infarct core and adjacent area, and thereafter slowed tissue and behavioral repair (FIG. 15F). In studies of ischemic stroke, A3AR plays a neuroprotective role in the central nervous system. Administration of an A3AR agonist before global brain ischemia improved post-ischemic cerebral blood circulation, survival, and neuronal preservation. However, the role of this signaling axis during the progression of WM ischemia/VaD has not been determined.

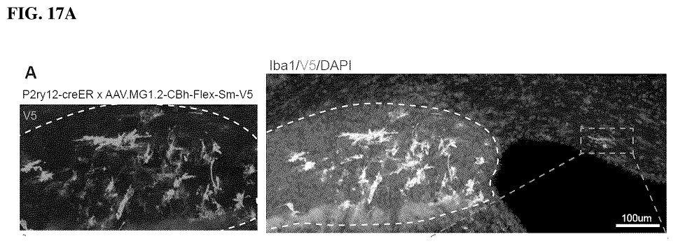

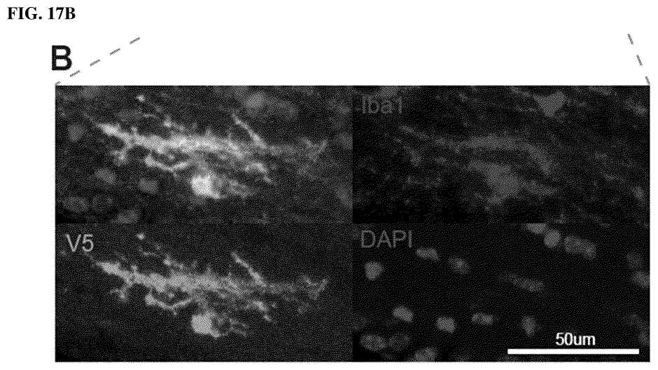

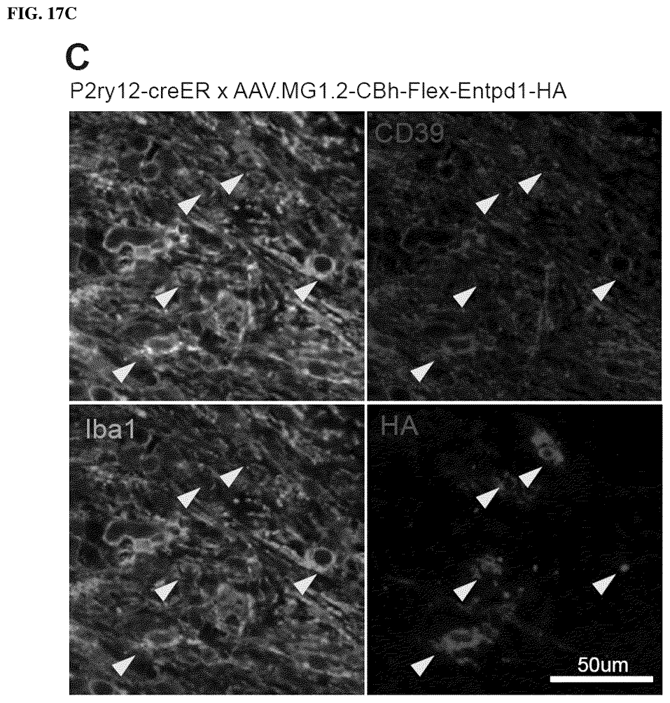

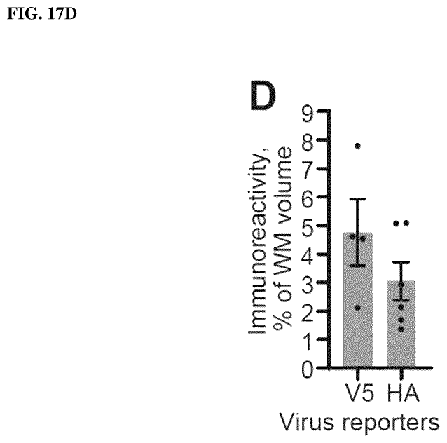

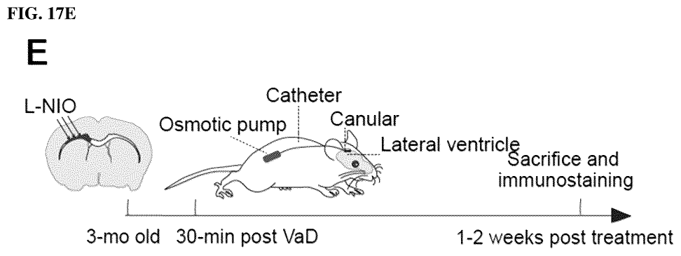

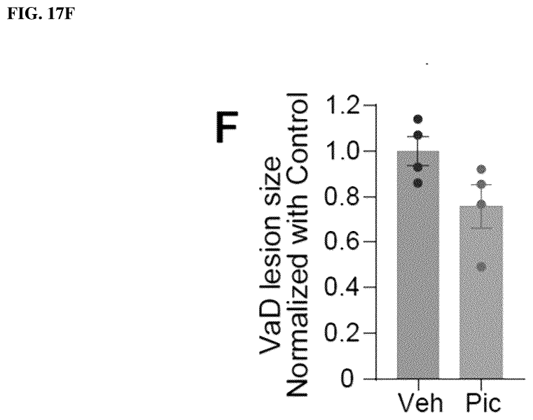

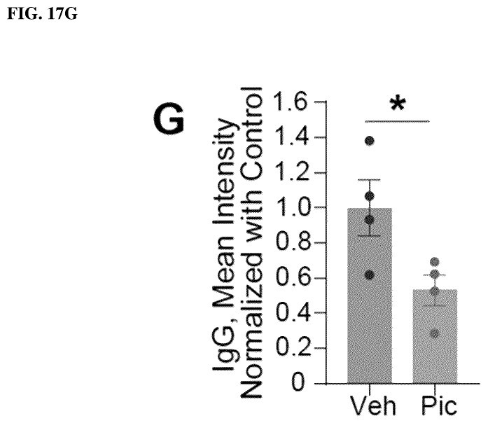

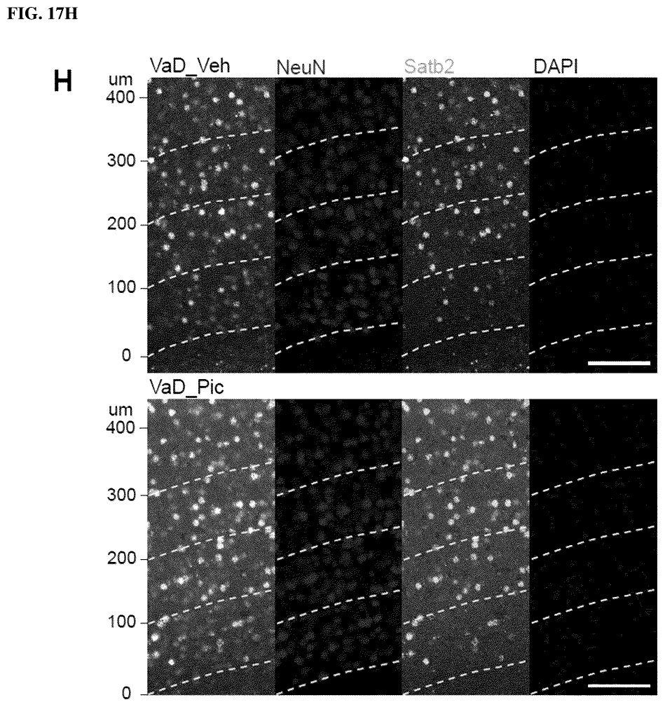

To better understand the mechanism of how CD39-A3AR signaling regulates VaD recovery, the expression of microglia CD39 was specifically upregulated using intersecting virus and transgenic mouse approaches and its effect was compared on lesion sizes with control virus (FIG. 15G), to specifically target microglia at a time period well after the induction of VaD, and during the process of VaD progression. This approach was able to efficiently label microglia in lesion core and adjacent area (FIGS. 17A and 17B). Overexpression of CD39 in microglia was validated by HA staining (FIG. 17C). Both control (V5 labeled) and CD39 overexpression (HA labeled) viruses showed decent efficacy (FIG. 17D). The core sizes were significantly reduced in the CD39 overexpression group (FIGS. 15H and 151). The length of lesion core across the anterior-posterior axis was significantly reduced from 800- to 450-μm (FIG. 15J). These results may indicate that CD39 overexpression in microglia (induced 5-10 days before VaD induction) showed mixed neural protective and neural repair effects in VaD tissue recovery.

To investigate whether increasing the affected CD39-A3AR signaling is beneficial to VaD recovery, piclidenoson, an A3AR-specific agonist, was delivered either acutely or in a delayed manner after VaD induction. Piclidenoson is 50-fold more potent in A3AR action than with A1AR and A2bAR, and has been safely used in humans in recent phase III clinical trials for psoriasis. Acute administration of piclidenoson 30-min post stroke, for 1-2 weeks, by direct infusion into the lateral ventricle, significantly reduced blood brain barrier (BBB) leakage and may reduce lesion size (FIGS. 17E-17G).

Unlike large artery stroke, periventricular WM microinfarcts in the human are often asymptomatic and progress for months to years before development of VaD symptoms, so that any medication in VaD is likely to be a delayed treatment. Delayed (5 days after VaD induction) piclidenoson delivery (FIG. 15K) produced a significant reduction of lesion size (FIG. 15L). Although measures of axonal projections were not changed (FIG. 15M), markers of myelination were significantly increased in affected WM by delayed piclidenoson treatment (FIG. 15N). Importantly, the NOR memory test showed that piclidenoson significantly reduced memory deficits in VaD animals (FIG. 15P), which also included restored c-Fos expression in PFC (AC) and HPC (CA2/3) (FIGS. 15Q and 15R). Human VaD is associated with gait and motor abnormalities that predict dementia status and are associated with morbidity. Using a grid walking task, which tests limb motor control, delayed piclidenoson treatment successfully rescued the motor deficit in VaD (FIGS. 15S and 15T). The reduced expression of neuronal TFs, Satb2 and Cux1, in the Layer VI of motor somatosensory cortex, adjacent to the WM VaD lesion is a marker of secondary injury effect in this model (FIGS. 5D-5J). Immunostaining using the animal cohorts after grid-walking tests showed that the reduced Satb2 and Cux1 expression were significantly ameliorated by delayed piclidenoson treatment (FIGS. 15U, 15V, and 17H). To the best of our knowledge, this is the first finding of a drug that reduces lesion progression or enhances tissue repair in such a delayed treatment.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating or preventing an ischemic disease, the method comprising administering to a subject an A3AR agonist, or a pharmaceutically acceptable salt thereof.

2. The method of claim 1, wherein the ischemic disease is cerebral ischemia.

3. The method of claim 1 or 2, wherein the ischemic disease is dementia.

4. The method of any one of claims 1-3, wherein the ischemic disease is vascular dementia.

5. The method of claim 4, wherein the subject has Alzheimer's disease.

6. The method of any one of claims 1-5, wherein the method reduces blood-brain barrier leakage.

7. The method of any one of claims 1-6, wherein the A3AR agonist, or a pharmaceutically acceptable salt thereof is an A3AR-specific agonist.

8. The method of any one of claims 1-7, wherein the A3AR agonist is CF101 or CF102.

9. The method of any one of claims 1-8, wherein the A3AR agonist is CF101.

10. The method of any one of claims 1-8, wherein the A3AR agonist is CF102.

11. The method of any one of claims 1-10, wherein the A3AR agonist is a pharmaceutically acceptable salt of CF101.

12. The method of any one of claims 1-11, wherein the method reduces lesion size.

13. The method of claim 12, wherein the lesion size is reduced by about 1.6-fold to about 5-fold.

14. The method of any one of claim 1-13, wherein the method reduces lesion progression.

15. The method of claim 14, wherein the lesion size progression is reduced by about 1.6-fold to about 5-fold.

16. The method of any one of claim 1-15, wherein the method enhances tissue repair.

17. The method of claim 16, wherein tissue repair enhancement comprises about a 1.3-fold increase in white matter structure.

18. The method of any one of claims 1-17, wherein the method reduces memory deficits.

19. The method of claim 18, wherein memory deficits are reduced by about 1.3-fold.

20. The method of any one of claims 1-19, wherein the method reduces motor deficit.

21. The method of any one of claims 1-20, wherein motor deficit is reduced by about 2-fold.

22. The method of any one of claims 1-21, wherein the method increases expression of one or more neuronal transcription factors adjacent to one or more vascular dementia lesions.

23. The method of claim 22, wherein the expression of one or more neuronal transcription factors adjacent to one or more vascular dementia lesions is increased by about 1.2-fold.

24. The method of any one of claims 1-23, wherein the method increases expression of Satb2 and Cux1.

25. The method of claim 24, wherein Satb2 expression is increased by about 1.1-fold.

26. The method of claim 24 or 25, wherein Cux1 expression is increased by about 1.2-fold.

27. A method of treating or preventing an ischemic disease, the method comprising modulating Serpine2 activity.

28. The method of claim 27, wherein the modulating Serpine2 activity comprises downregulating Serpine2 activity.

29. The method of claim 27 or 28, wherein the ischemic disease is a cerebral ischemia.

30. The method of claim 27 or 28, wherein the ischemic disease is dementia.

31. The method of claim 27 or 28, wherein the ischemic disease is vascular dementia.

32. The method of claim 27 or 28, wherein the subject has Alzheimer's disease.

33. The method of any one of claims 27-32, wherein the method reduces memory deficits.

34. The method of claim 33, wherein memory deficits are reduced by about 1.45-fold.

35. The method of any one of claims 27-34, wherein the method promotes myelination.

36. The method of claim 35, wherein promoting myelination comprises an about 2.07-fold increase in myelination.

37. The method of any one of claims 27-36, wherein the method promotes tissue repair.

38. The method of claim 37, wherein promoting tissue repair comprises an about 2.4-fold increase in myelination cell replacement and about a 2.07-fold increase in myelination.

39. The method of any one of claims 27-38, wherein the method promotes memory recovery.

40. The method of claim 39, wherein promoting memory recovery comprises an about 1.45-fold increase in memory recovery.
